(19) United States
(12) Patent Application Publication
(10) Pub. No.: US 2001/0034445 A1
(43) Pub. Date: Oct. 25, 2001

(54) Vitronectin receptor antagonists

(75) Inventors: Fadia E. Ali, Cherry Hill, NJ (US); William E. Bondinell, Wayne, PA (US); Richard M. Keenan, Malvern, PA (US); Thomas Wen-Fu Ku, Dresher, PA (US); William H. Miller, Schwenksville, PA (US); James Samanen, Phoenixville, PA (US)

Correspondence Address: GLAXOSMITHKLINE, Corporate Intellectual Property - UW2220, P.O. Box 1539, King of Prussia, PA, 19406-0939, US

(73) Assignee: SmithKline Beecham Corporation

(21) Appl. No.: 09/769,125

(22) Filed: Jan. 24, 2001

Related U.S. Application Data
(63) Continuation of application No. 09/091,936, filed on Dec. 3, 1998, now abandoned.
Application No. 09/091,936 is a 371 of international application No. PCT/US96/20744, filed on Dec. 20, 1996.
(60) Provisional application No. 60/009,532, filed on Dec. 29, 1995.

(51) Int. Cl. C07D 241/20; C07D 239/12

(52) U.S. Cl. 544/326; 544/330; 544/336

(57) ABSTRACT

Compounds of formula (I) are disclosed which are vitronectin receptor antagonists useful in the treatment of osteoporosis.

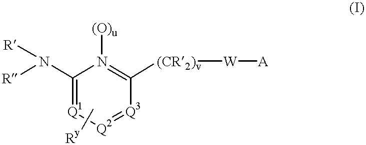

### FIELD OF THE INVENTION

This invention relates to pharmaceutically active compounds which inhibit the vitronectin receptor and are useful for the treatment of diseases wherein ihibition of the vitronectin receptor is indicated, such as inflammation, cancer, angiogenesis, atherosclerosis, restenosis, and diseases wherein bone resorption is a factor.

### BACKGROUND OF THE INVENTION

Integrins are a superfamily of cell adhesion receptors, which are transmembrane glycoproteins expressed on a variety of cells. These cell surface adhesion receptors include gpIIb/IIIa, the fibrinogen receptor, and αᵥβ₃, the vitronectin receptor. The fibrinogen receptor gpIIb/IIIa is expressed on the platelet surface and it mediates platelet aggregation and the formation of a hemostatic clot at the site of a bleeding wound. Philips, et al., Blood., 1988, 71, 831.

The vitronectin receptor αᵥβ₃ is expressed on a number of cells, including endothelial, smooth muscle, osteoclast, and tumor cells, and, thus, it has a variety of functions. The αᵥβ₃ receptor expressed on the membrane of osteoclast cells is believed to play a role in the bone resportion process and contribute to the development of osteoporosis. Ross, et al., J. Biol. Chem., 1987, 262, 7703; Fisher, et al., Endocrinology 1993, 132, 1411; Bertolini et al., J. Bone Min. Res., 6, Sup. 1, S146, 252; EP 528 587 and 528 586. The αᵥβ₃ receptor expressed on human aortic smooth muscle cells stimulates their migration into neointima, which leads to the formation of atherosclerosis and restenosis after angioplasty. Brown, et al., Cardiovascular Res., 1994, 28, 1815. Additionally, a recent study has shown that a αᵥβ₃ antagonist is able to promote tumor regression by inducing apoptosis of angiogenic blood vessels. Brooks, et al., Cell, 1994, 79, 1157. Thus, agents that would block the vitronectin receptor would be useful in treating diseases mediated by this receptor, such as osteoporosis, atherosclerosis, restenosis and cancer.

Alig et al., EP 0 381 033, Hartman, et al., EP 0 540,334, Blackburn, et al., WO 93/08174, Bondinell, et al., WO 95/18619, Bondinell, et al., WO 94/14776, Blackburn, et al. WO 95/04057, Egbertson, et al, EP 0 478 328, Sugihara, et al. EP 529,858, Porter, et al., EP 0 542 363, and Fisher, et al., EP 0 635 492, and many others disclose certain compounds that are useful for selectively inhibiting the fibrinogen receptor. PCT/US95/08306, filed Jun. 29, 1995 (SmithKline Beecham Corp.) and PCT/US95/08146 filed Jun. 29, 19951995 (SmithKline Beecham Corp.) disclose vitronectin receptor selective antagonists. However, there are few reports of compounds which are potent vitronectin receptor antagonists. It has now been discovered that certain appropriately substituted amino pyridine compounds are potent inhibitors of the vitronectin receptor. In particular, it has been discovered that the amino pyridine moiety may be combined with a fibrinogen atagonist template to prepare compounds which are more potent inhibitors of the vitronectin receptor than the fibrinogen receptor.

### SUMMARY OF THE INVENTION

This invention comprises compounds of the formula (I) as described hereinafter, which have pharmacological activity for the inhibition of the vitronection receptor and are useful in the treatment of inflammation, cancer, cardiovascular disorders, such as atherosclerosis and restenosis, and diseases wherein bone resorption is a factor, such as osteoporosis.

This invention is also a pharmaceutical composition comprising a compound according to formula (I) and a pharmaceutically acceptable carrier.

This invention is also a method of treating diseases which are mediated by the vitronectin receptor. In a particular aspect, the compounds of this invention are useful for treating atherosclerosis, restenosis, inflammation, cancer and osteoporosis.

### DETAILED DESCRIPTION

This invention comprises novel compounds which are more potent inhibitors of the vitronectin receptor than the fibrinogen receptor. The compounds of the instant invention comprise a fibrinogen receptor antagonist template that is linked to an optionally substituted o-amino pyridine moiety according to formula (I):

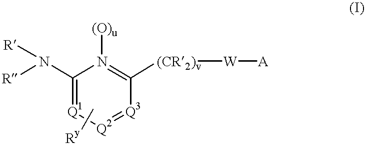

wherein

A is a fibrinogen antagonist template;

W is a linking moiety of the form —(CHR^{g})ₐ—U—(CHR^{g})_{b}—V—;

Q¹, Q² and Q³ are independently N or C—R^{y}, provided that no more than one of Q¹, Q² and Q³ is N;

R′ is is H or C₁₋₆alkyl, C₃₋₇cycloalkyl-C₀₋₆alkyl or Ar—C₀₋₆alkyl

R″ is R′, —C(O)R′ or —C(O)OR′;

R^{g} is H or C₁₋₆alkyl, Het-C₀₋₆alkyl, C₃₋₇cycloalkyl-C₀₋₆alkyl or Ar—C₀₋₆alkyl;

R^{k} is R^{g}, —C(O)R^{g} or —C(O)OR^{g}

Rⁱ is H, C₁₋₆alkyl, Het-C₀₋₆alkyl, C₃₋₇cycloalkyl-C₀₋₆alkyl, Ar—C₀₋₆alkyl, Het-C₀₋₆alkyl-U′-C₁₋₆alkyl, C₃₋₇cycloalkyl-C₀₋₆alkyl-U′-C₁₋₆alkyl or Ar—C₀₋₆alkyl-U′-C₁₋₆alkyl;

R^{y} is H, halo, —OR^{g}, —SR^{g}, —CN, —NR^{g}R^{k}, —NO₂, —CF₃, CF₃S(O)ᵣ—, —CO₂R^{g}, —COR^{g} or —CONR^{g}₂;

U and V are absent or CO, CR^{g}₂, C(═CR^{g}₂), S(O)_{c}, O, NR^{g}, CR^{g}O^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}C(S), S(O)₂NR^{g}, NR^{g}S(O)₂ N═N, NR^{g}NR^{g}, NR^{g}CR^{g}₂, NR^{g}CR^{g}₂, CR^{g}₂O, OCR^{g}₂, CR^{g}═CR^{g}, C≡C, Ar or Het;

a is 0, 1 or 2;

b is 0, 1 or 2;

c is 0, 1 or 2;

u is 0 or 1;

v is 0 or 1;

and pharmaceutically acceptable salts thereof-,

provided that:

(i) when v is 0, and R′, R″ and R^{y} are H, and Q¹-Q³ are CH, W-A is not 7-aminocarbonyl-2,3,4,5-tetrahydro-3-oxo-4-methyl-1H-1,4-benzodiazepine-2-acetic acid, 7-aminocarbonyl-1-acetyl-2,3,4,5-tetrahydro-3-oxo-4-methyl-1H-1,4-benzodiazepine-2-acetic acid, or 7-aminocarbonyl-2,3,4,5-tetrahydro-3-oxo4-methyl-1H-1-benzazepine-2-acetic acid, or the methyl esters thereof;

(ii) when v is 0 or 1 and R′, R″ and R^{y} are H, and Q¹-Q³ are CH, W-A is not 3-propanoyl-glycyl-aspartyl-phenylalanine, or

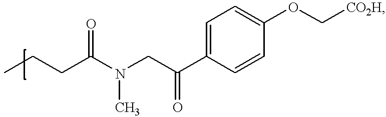

and the benzyl esters thereof.

Preferably, Q¹, Q² and Q³ are all CH, and u is 0.

Suitably, R′ is H and R″ is H or C₁₋₄alkyl.

Preferably v is 1.

Suitably, W is —(CHR^{g})ₐ—CONRⁱ— or —(CHR^{g})ₐ—NRⁱCO—

Suitably U′ is CONR′ or NR′CO.

A fibrinogen receptor antagonist is an agent that inhibits the binding of fibrinogen to the platelet-bound fibrinogen receptor GPIIb-IIIa. Many fibrinogen antagonists are known to the art. As used herein, the term “fibrinogen receptor antagonist template” means the core structure of a fibrinogen receptor antagonist, said core containing an acidic group and being linked to an organic group substituted with a basic nitrogen moiety. Typically, the core structure adds some form of rigid spacing between the acidic moiety and the basic nitrogen moiety, and contains one or more ring structures or amide bonds to effect this. It is preferred that about twelve to fifteen, more preferably thirteen or fourteen, intervening covalent bonds via the shortest intramolecular path will exist between the acidic group of the fibrinogen receptor antagonist template and nitrogen of the pyridine moiety in formula (I). It is an object of this invention that a fibrinogen receptor antagonist is converted to a vitronectin receptor antagonist by replacing the basic nitrogen group in a fibrinogen receptor antagonist with an optionally substituted o-amino pyridine group. In addition, the number of intervening covalent bonds between the acidic moiety and the nitrogen of the pyridine will be about two to five, preferably three or four, covalent bonds shorter than the number of intervening covalent bonds between the acidic moiety and the basic nitrogen group of the fibrinogen antagonist. The identity of the linking moiety W may be chosen to obtain the proper spacing between the acidic moiety of the fibrinogen antagonist template and the nitrogen atom of the pyridine. Generally, a fibrinogen antagonist will have an intramolecular distance of about 16 angstroms between the acidic moiety (e.g., the atom which gives up the proton or accepts the electron pair) and the basic moiety (e.g., which accepts a proton or donates and electron pair), while the vitronectin antagonist will have about 14 angstroms between the respective acidic and basic centers.

For purposes of illustration, using the 7-2,3,4,5-tetrahydro-3-oxo4methylbenzodiazepine fibrinogen antagonist template disclosed in WO 93/08174 as a suitable fibrinogen antagonist template, the compound (R,S)-7-([[4-(aminoiminomethyl)phenyl]amino]carbonyl]-4-(2-phenylethyl)-1,3,4,5-tetrahydro-3-oxo-2H-1,4-benzodiazepine-2-acetic acid, which is potent and selective fibrinogen antagonist, is converted to a potent and selective vitronectin receptor antagonist by replacing the 4-(aminoiminomethyl)phenyl moiety with the 6-amino-pyrid-2-yl moiety. As illustrated below in FIG. 1, in the former case, there are sixteen intervening covalent bonds between the acidic moiety and the basic moiety; in the fibrinogent antagonist whereas, in the latter case there are 13 intervening covalent bonds in the vitronectin antagonist of this invention.

FIG. 1

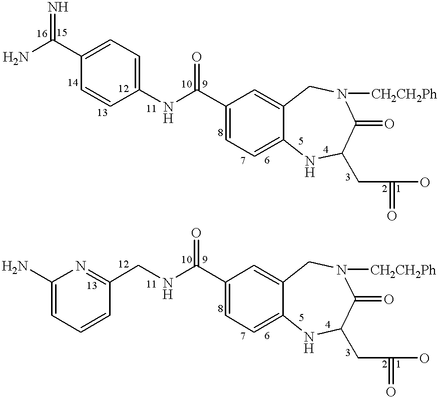

In fact the 4-(aminoiminomethyl)phenyl moiety is a common substituent on fibrinogen antagonist templates known to the art, and simple replacement of this moiety with an optionally substituted (6-aminopyrid-2-yl)methyl moiety may serve as guide to converting compounds having known fibrinogen antagonist templates into vitronectin receptor antagonists.

Also included in this invention are pharmaceutically acceptable addition salts, complexes or prodrugs of the compounds of this invention. Prodrugs are considered to be any covalently bonded carriers which release the active parent drug according to formula (I) in vivo. In cases wherein the compounds of this invention may have one or more chiral centers, unless specified, this invention includes each unique nonracemic compound which may be synthesized and resolved by conventional techniques. In cases in which compounds have unsaturated carbon-carbon double bonds, both the cis (Z) and trans (E) isomers are within the scope of this invention. In cases wherein compounds may exist in tautomeric forms, such as keto-enol tautomers, such as

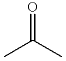

and

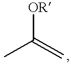

and each tautomeric form is contemplated as being included within this invention whether existing in equilibrium or locked in one form by appropriate substitution with R′.

The compounds of formula (I) inhibit the binding of vitronectin and other RGD-containing peptides to the vitronectin (αᵥβ₃) receptor. Inhibition of the vitronectin receptor on osteoclasts inhibits osteoclastic bone resorption and is useful in the treatment of diseases wherein bone resorption is associated with pathology, such as osteoporosis. Additionally, since the compounds of the instant invention inhibit vitronectin receptors on a number of different types of cells, said compounds would be useful in the treatment of inflammation and cardiovascular diseases, such as atherosclerosis and restenosis, and would be useful as anti-metastatic and antitumor agents.

Table I, below, describes certain fibrinogen receptor antagonists, whose core structures are useful in carrying out the instant invention. Reference should be made to the patent applications and other publications for their full disclosures, including the methods of preparing said templates and specific compounds embodying said templates. The entire disclosure of the noted patent applications and other publications is incorporated herein by reference as though fully set forth. The list following is not intended to limit the scope of the present invention, but only to illustrate certain known templates.

TABLE I
Adir et Compagnie
FR 928004, June 30, 1992, Fauchere, et al.
EP 0578535, June 29, 1993, Fauchere, et al.
CA 2128560, Jan. 24, 1995, Godfroid, et al.
Asahi Breweries, Ltd.
JP 05239030, Sep. 17, 1993.
Asahi Glass
WO 90/02751, Ohba, et al., Sept. 8, 1989.
WO 90/115950, Mar. 22, 1990, Ohba, et al.
EP 0406428, Jan. 9, 1991.
WO 92/09627, Isoai, A. et al., Nov. 29, 1991.
Cassella AG
DE 4207254, (Der 93-289298/37) Mar. 7, 1992, Zoller, et al.
EP 93904010, Feb. 24, 1993.
EP 0565896, Mar. 18, 1993, Klinger, et al.
EP 0566919, (Der 93-338002/43) Apr. 3, 1993, Zoller, et al.
EP 580008, (Der 94-027663/04) July 6, 1993, Zoller, et al.
DE 224414, July 6, 1993, Zoller, et al.
EP 584694, (Der 94-067259/09) Apr. 2, 1994.
DE 4301747, (Der 94-235891/29) Jul. 28, 1994, Zoller, et al.
DE 4308034, (Der 94-286666/36) Sept. 15, 1994, Klinger, O. et al
DE 4309867, Sept. 29, 1994, Klingler, et al.
Chiron
WO 93/07169, (Der 93-134382/16), Mar. 15, 1993, Devlin, et al.
Ciba Geigy
EP 0452210, (Der 91-305246/42) Apr, 5, 1990, Describes
aminoalkanoyl-GDF analogs.
EP 0452257, Mar. 26, 1991, Allen, et al.: Describes
aminoalkanoylAsp-Phe analogs.
COR Therapeutics
WO 90/15620, June 15, 1990.
EP 0477295, Apr. 1, 1992, Scarborough, et al.
WO 92/08472, May 29, 1992, Scarborough, et al.
WO 93/223356, April 27, 1993, Swift, et al.
EP 0557442, Sept. 1, 1993, Scarborough, et al.
Scarborough, et al., J. Biol. Chem., 266, 9359, 1991.
Daiichi Pharm Co Ltd.
JP 05078344-A, (Der 93-140339/17) Mar. 30, 1993.
DuPont Merck
WO 93/07170, Apr. 15, 1993.
WO 94/11398, May 26, 1994: Wells, et al.
IL 109237, Jul. 31, 1994.
WO 94/22909, (Der 94-333113/41) Oct. 13, 1994, DeGrado, et al.
WO 94/22910, (Der 94-333114/41 Oct. 13, 1994: DeGrado, et al.
WO 94/22494, (Der 94-332838/41) Oct. 13, 1994: DeGrado, et al.
EP 625164, Nov. 23, 1994, Degrado, et al.
Mousa, et al, Circulation, 89, 3, 1994.
Jackson, J. Amer. Chem. Soc., 116, 3220, 1994.
Ellem Ind Farma Spa
GB 2207922, Aug, 3, 1988.
Farmitalia Erba SRL Carlo
EP 611765 (Der 94-265375/33), Aug 24, 1994: Cozzi, et al.
Fuji Photo Film
JP 04208296-A (Der. 92-303598/38), Nov. 30, 1990.
JP 04213311-A (Der. 92-305482/38), Nov. 27, 1990.
JP 04217693-A, (Der. 92-312284/38), Oct. 23, 1990.
JP 04221394-A (Der. 92-313678/38), Oct. 26, 1990.
JP 04221395-A (Der. 92-313679/38), Oct. 26, 1990.
JP 04221396-A (Der. 92-313680/38), Oct. 26, 1990.
JP 04221397-A (Der. 92-313681/38), Dec. 20, 1990.
EP 0482649 A2, April 29, 1992, Kojima, et al..
EP 0488258A2, June 3, 1992, Komazawa, et al..
EP 503301-A2, Feb. 14, 1991, Kitaguchi, et al..
JP 05222092, May 21, 1993, Nishikawa, et al..
JP 06239885, (Der 94-313705/39), Aug 30, 1993, Nishikawa, et al.
WO 9324448, (Der 93-405663/50), Dec. 9, 1993, Nishikawa, et al.
JP 06228189, (Der 94-299801/37), Aug. 16, 1994.
EP 619118, (Der 94-311647/39), Oct. 12, 1994, Nishikawa, et al..
Fujisawa
EP 0513675, May 8, 1992, Umekita, et al.
WO 9409030-A1, Apr. 28, 1994, Takasugi, et al.
EP 0513675, (Der 92-383589/47).
WO 9500502, Jan, 5, 1995, Oku, et al.
FR 144633: Thromb Haem. 69, 706, 1993.
Cox, et al., Thromb. Haem., 69, 707, 1993.
Genentech
WO 90/15072 (Der 91007159.
WO 91/01331 (Der 91058116), July 5, 1990, Barker, et al.
WO 91/04247, Sept. 24, 1990, Webb.
WO 91/11458 (Der 91252610), Jan. 28, 1991, Barker, et al.
WO 92/07870, Oct. 24, 1991, Burnier, et al.
WO 92/17492, Oct. 15, 1992, Burnier, et al.
CA 2106314, Oct. 6, 1992, Burnier, et al.
WO 93/08174, Oct. 15, 1991, Blackburn, et al.
CA 2106314, Oct. 6, 1992, Burnier, et al.
EP 0555328, Aug. 18, 1993, Burnier, et al.
WO 95/04057, Feb. 9, 1995, Blackburn, et al.
Scarborough, et al., J. Biol. Chem. 268, 1066, 1993.
Dennis, et al., Proc. Natl. Acad. Sci. USA, 87, 2471, 1989.
Barker, et al., J. Med. Chem., 35, 2040, 1992.
McDowell; Gadek, T. R., J. Amer. Chem. Soc., 114, 9245, 1992.
Glaxo
EP 537980, Oct. 13, 1992, Porter, et al.
EO 0542363, Nov. 10, 1992, Porter, et al.
WO 93/22303, Jan. 11, 1993, Middlemiss, et al.
WO 93/22303, Jan. 11, 1993, Middlemiss, et al.
WO 93/14077, Jan. 15, 1993, Porter, et al.
EP 609282 A1, Aug. 10, 1994, Porter, et al.
EP 612313, Aug. 31, 1994, Porter, et al.
EP 93911769, Apr. 20, 1994, Midlemiss, et al.
EP 637304 A1, Feb. 8, 1995, Middlemiss, et al.
Hann, et al., “An Investigation of the Bioactive Conformation of
ARG-GLY-ASP Containing Cyclic Peptides and Snake Venom Peptides
Which Inhibit Human Platelet Aggregation, ” In Molecular Recognition,
Chemical and Biochemical Problems II”, S. M. Roberts, Ed., The Royal
Society of Chemistry, Cambridge, 1992.
Ross, B. C., “Nonpeptide Fibrinogen Receptor Antagonists,” In
Seventh RSC-SCI Medicinal Chemistry Symposium, The Royal Society of
Chemistry Fine Chemicals and Medicinals Group and SCI Fine Chemicals
Group, Churchill College, Cambridge, 1993, L20.
Pike, et al., Thromb. Haem., 69, 1071, 1993.
Hoechst
DE 4009506, Mar. 24, 1990, Konig, et al.
Hoffman-La Roche
AU 9344935, (Der 94-118783/15), Mar. 10, 1994.
EP 0592791, Apr. 20, 1994, Bannwarth, et al..
Kogyo Gijutsuin
JP 06179696, June 28, 1994.
Kyowa Hakko Kogyo KK
JP 05078244-A, Mar. 30, 1993.
Laboratoire Chauvin
WO 9401456, Jan. 20, 1994, Regnouf, et al.
La Jolla Cancer Res. Fndn
WO 9500544, Jan. 5, 1994, Pierschbacher, et al.
US 079441, Jan 5, 1994, Pierschbacher, et al.
Lilly/COR Therapeutics
EP 0635492, Jan. 25, 1995, Fisher, et al.
Medical University of South Carolina
EP 587770, Mar. 23, 1994, Halushka, et al.
Merck
EP 0368486 (Der 90-149427/20), Nov. 10, 1988.
EP 0382451 (Der 90248531).
EP 0382538 (Der 90248420).
EP 0410537, July 23, 1990, Nutt, et al..
EP 0410539, July 25, 1990, Nutt, et al..
EP 0410540, July 25, 1990, Nutt, et al.
EP 0410541, July 25, 1990, Nutt, et al.
EP 0410767, July 26, 1990, Nutt, et al.
EP 0411833, July 26, 1990, Nutt, et al.
EP 0422937, Oct. 11, 1990, Nutt, et al.
EP 0422938, Oct. 11, 1990, Nutt, et al.
EP 0487238, October 13, 1991, Connolly, et al.
EP 0437367 (Der 91209968), Sato et al.
EP 576898, Jan. 5, 1994, Jonczyk, et al.
WO 9409029, Apr. 28, 1994, Nutt, et al.
EP 618225, (Der 94-304404/38) Oct. 5, 1994.
DE 4310643, (Der 94-311172/39), Oct. 6, 1994, Jonczyk, et al.,
Describes cyclic RGD analogs as antimetastatic agents.
NO 9404093, Oct. 27, 1994, Jonczyk, et al.
EP 0632053, Jan. 4, 1995, Jonczyk, et al.
EP 0479481, Sept. 25, 1991, Duggan et al.
EP 0478328, Sept. 26, 1991, Egbertson, et al.
EP 0478362, Sept. 27, 1991, Duggan et al.
EP 0478363, Sept. 27, 1991, Laswell, et al.
EP 0512829, May, 7, 1992, Duggan, et al.
EP 0512831, May, 7, 1992, Duggan, et al.
EP 0528586, August 5, 1992, Egbertson, et al.
EP 0528587, August 5, 1992, Egbertson, et al.
EP 0540334, October 29, 1992, Hartman, et al.
US 5227490, Feb. 21, 1992, Hartman, et al.
CA 2088518, Feb. 10, 1993, Egbertson, et al.
US 5206373-A, (Der 93-151790/18) Apr. 27, 1993, Chung, et al.
WO 9316994, (Der 93-288324/36), Sep. 2, 1993, Chung, et al.
US 5264420-A, Nov. 23, 1993.
US 5272158, Dec. 21, 1993, Hartman, et al.
US 5281585, Jan. 25, 1994, Ihle, et al.
GB 945317 A, Mar. 17, 1994.
GB 2271567 A, Apr. 20, 1994, Hartman, et al.
US 5294616, (Der 94-091561/11) Mar. 15, 1994, Egbertson, et al.
US 5292756, (Der 94-082364) Apr. 8, 1994, Hartman, et al.
WO 9408577, Apr. 28, 1994, Hartman, et al.
WO 9408962, Apr. 28, 1994, Hartman, et al.
WO 9409029, (Der 94-151241/18) Apr. 28, 1994, Hartman, et al.
US 5312923, May 17, 1994, Chung, et al.
HU 9400249, May 30, 1994, Gante, et al.
WO 9412181, (Der 94-199942/24), Jun. 9, 1994, Egbertson, et al.
US 5321034, June 14, 1994, Duggan, et al.
US 5334596, Aug. 2, 1994, Hartman, et al.
EP 0608759 A, Aug. 3, 1994, Gante, et al.
WO 9418981, (Der 94-293975/36) Sep. 1, 1994, Claremon, et al.
GB 2276384, (Der 94-287743/36) Sep. 28, 1994, Claremon, et al.
WO 9422825, Oct. 13, 1994, Claremon, et al.
EP 0623615A, Nov. 9, 1994, Raddatz, et al.
WO 9504531, Feb. 16, 1995, Hartman, et al.Nutt, et al., Development
of Novel, Highly Selective Fibrinogen Receptor Antagonists as
Potentially Useful Antithrombotic Agents, In Peptides, Chemistry
and Biology, Proc. 12th Amer. Peptide Symp., J. A. Smith and
J. E. Rivier, Ed., ESCOM, Leiden, 1992; 914.
Hartman, et al., J. Med. Chem., 35, 4640, 1992.
Gould, et al., Thromb. Haem., 69, 539, 1993.
Merrell Dow
WO 93/24520, May 14, 1993, Harbeson., et al.
WO 9324520, Dec. 9, 1993, Harbeson, et al.
WO 9429349, Dec. 22, 1994, Harbeson, et al.
Nippon Steel Corp
WO 9405696, Mar. 17, 1993, Sato., et al,.
EP 628571, Dec. 14, 1994, Sato, et al.
WO 9501371, Jan. 12, 1995, Sato, et al.
ONO Pharmaceuticals
JP 05286922 (Der 93-383035/48).
Roche
EP 038, 362, Feb. 19, 1990, Muller, et al..
EP 0372486, June, 13, 1990, Allig, et al.
EP 0381033, July, 8, 1990, Allig, et al.
EP 0384362, August 29, 1990, Allig, et al.
EP 0445796, Sept. 11, 1991, Allig, et al.
EP 0505868, Sept. 30, 1992, Allig, et al.
US 5273982-A, (Der 94-006713/01) Dec. 28, 1993
Alig, et al., J. Med. Chem., 35, 4393, 1992.
Rhone-Poulenc Rorer
US 4952562, Sept. 29, 1989, Klein et al.
US 5064814, (Der 91-353169/48) Apr. 5, 1990
WO 9104746, Sept. 25, 1990, Klein et al.
WO 91/05562, Oct. 10, 1989, Klein et al.
WO 91/07976, (Der 91-192965) Nov. 28, 1990, Klein et al.
WO 91/04746, Klein et al.
WO 92/18117, Apr. 11, 1991, Klein et al.
US 5086069, (Der 92-064426/08) Apr. 2, 1992.
WO 92/17196, Mar. 30, 1992, Klein et al.
US 5328900, (Der 94-221950/27) Jul. 12, 1992.
US 5332726, (Der 94-241043/29) Jul. 26, 1994.
WO 93/11759, Dec. 7, 1992, Klein et al.
EP 0577775, Jan 12, 1994, Klein, et al.
CA 2107088, Sept. 29, 1992, Klein, et al.
Sandoz
EP 0560730, Mar. 8, 1993, Kottirisch, et al.
Kottirisch, et al., Biorg. Med. Chem. Lett 3, 1675-1680, 1993.
Schering AG
EP 530937, Mar. 10, 1993, Noeski-Jungblut, et al.
Searle/Monsanto
EP 0319506, (Der 89-3195506) Dec. 2, 1988, Adams, et al.
EP 0462, 960, June, 19. 1991, Tjoeng, et al.
US 4857508, Adams, et al.
EP 0502536, (Der 92-301855) Mar. 3, 1991, Garland, et al.
EP 0319506, Dec. 2, 1988, Adams et al.
US 4992463, Aug. 18, 1989.
US 5037808, Apr. 23, 1990.
EP 0454651 A2, Oct. 30, 1991, Tjoeng, et al..
US 4879313, July, 20, 1988.
WO 93/12074, Nov. 19, 1991, Abood, et al.
WO 93/12103, Dec. 11, 1991, Bovy, et al.
US 5091396, Feb. 25, 1992, Tjoeng, et al.
WO 92/15607, Mar. 5, 1992, Garland, et al.
WO 93/07867, Apr. 29, 1993, Bovy, et al.
US 888686, May 22, 1992, Bovy, et al.
CA 2099994, Sept. 7, 1992, Garland, et al.
US 5254573, Oct. 6, 1992, Bovy, et al.
(PF54C06), EP 0539343, Oct. 14, 1992, Bovy et al.
WO 93/12074, Nov. 27, 1992, Abood, et al.
WO 93/12103, Dec. 11, 1992, Bovy et al.
EP 0 539343, Apr. 28, 1993, Bovy, et al.
EP 0542708, May, 19, 1993, Bovy, et al.
WO 94/00424, June 23, 1993, Abood, et al.
WO 93/16038, Aug. 16, 1993, Miyano, et al.
WO 93US7975, Aug. 17, 1993, Zablocki, et al.
WO 93/18058, Sept. 16, 1993, Bovy, et al.
US 5254573, Oct. 19, 1993, Bovy, et al.
US, 5272162, Dec. 21, 1993, Tjoeng, et al.
EP 0574545, Dec. 22, 1993, Garland, et al.
WO 9401396, Jan. 20, 1994, Tjoeng, et al.
WO 9405694, (Der 94-101119/12) Mar. 17, 1994, Zablocki, et al.
US 5314902, May 24, 1994, Adams, et al.
WO 9418162, Aug, 18, 1994, Adams, et al.
WO 9419341, Sept. 1, 1994, Tjoeng, et al.
US 5344837, (Der 94-285503/35), Sept. 6, 1994, Zablocki, et al.
EP 614360, Sept. 14, 1994, Bovy, et al.
WO 9420457, (Der 94-302907/37) Sep. 15, 1994, Tjoeng, et al.
WO 9421602, (Der 94-316876/39), Sept. 29, 1994, Tjoeng, et al.
WO 9422820, Oct. 13, 1994, Abood, et al.
EP 630366, Dec. 28, 1994, Bovy, et al.
US 5378727, Jan. 3, 1995, Bovy, et al.
Fok, et al., Int. J. Peptide Prot. Res., 38, 124-130, 1991.
Zablocki, et al., J. Med. Chem., 35, 4914-4917, 1992.
Tjoeng, et al., Peptide Mimetics of the RGD Sequence, In Peptides,
Chem. and Biol. Proc. 12th Amer. Peptide Symp., J. A. Smith and
J. E. Rivier, Ed., ESCOM, Leiden, 1992; 752.
Nicholson, et al., Thromb. Haem., 69, 975, 1993.
SmithKline Beecham
EP 341 915, Ali, et al.
EP 425 212, Ali, et al.
EP 557 406 Callahan, et al.
WO 93/09133, Callahan, et al.
WO 93/00095, Bondinell, et al.
WO 94/14776, Bondinell, et al.
WO 95/18619, Bondinell, et al.
WO 94/12478, Keenan, et. al.
WO 94/12478, Callahan, et. al.
WO 94/12478, Callahan, et. al
WO 94/12478, Samanen, et. al.
Sumitomo Pharm. Co. Ltd.
WO 9501336, June 6, 1994, Ikeda, et al.
Sumitomo Seiyaku KK
JP 06025290, (Der 94-077374/10) Feb. 1, 1994.
Taisho Pharm. (Teijin, Ltd)
JP 05230009, (Der 93-317431/40, Feb. 24, 1992.
JP 9235479, Feb. 24, 1992.
WO 94/17804, Aug. 18, 1994, Mizushima.
EP 634171, Jan. 18, 1995, Nizushima
Takeda
EP 0529858, Apr. 3, 1993, H. Sugihara, et al.
EP 606881, Jul. 20, 1994.
EP 614664, Sept. 14, 1994, Miyake, et al.
Tanabe
WO 89/07609, Lobl, et al.
WO 92/00995, July 9, 1991, Lobl, et al.
WO 93/08823, Nov. 6, 1991, McKenzie
CA 2087021, Jan 10, 1991, Lobl, et al.
WO 92/08464, Nov. 15, 1991, McKenzie, et al.
Telios/La Jolla Cancer Research
US. 4578079, Nov. 22, 1983, Ruoslahti, et al.
US. 4614517, June 17, 1985, Ruoslahti, et al.
US. 4792, 525, June 17, 1985, Ruoslahti, et al.
US 4879237, (Der 90-154405/20) May, 24, 1985
WO 91/15515, (Der 91-325173/44) Apr. 6, 1990
US. 5041380, 1991, Ruoslahti, et al.
WO 95/00544 Jan. 5, 1995, Craig, et. al.
Cheng, et al., J. Medicin. Chem., 37, 1, 1994.
Collen, et al., 71, 95, 1994.
Temple University
WO 9409036, (Der 94-151248/18), Apr. 28, 1994.
Terumo KK
JP 6279389, Oct. 4, 1994, Obama, et al.
Karl Thomae/Boehringer Ingelheim
EP 0483667, May 6, 1992, Himmelsbach, et al.
EP 0496378, Jan. 22, 1992, Himmelsbach, et al.
EP 0503548, Sep. 16, 1992, Himmelsbach, et al.
AU A-86926/91, May 7, 1992, Himmelsbach, et al.
EP 0528369, Feb. 24, 1993, Austel, et al.
EP 0537696, Apr. 21, 1993 Linz, et al.
DE 4124942, Jan. 28, 1993, Himmelsbach, et al.
DE 4129603, Mar. 11, 1993, Pieper, et al.
EP 0547517 A1, (Per 93-198544) June 23, 1993, Soyka, et al.
EP 0567966, Nov. 3, 1993, Himmelsbach, et al.
EP 0567967, Nov. 3 1993, Weisenberger, et al.
EP 0567968, Nov. 3, 1993, Linz, et al.
EP 0574808, June 11, 1993, Pieper, et al.
Der 93-406657/51, Austel, et al.
EP 587134, (Per 94-085077/11) Mar. 16, 1994, Himmerlsbach, et al.
EP 589874, Apr. 6, 1994, Grell, et al.
(P534005), DE 4234295, Apr. 14, 1994, Pieper, et al.
EP 0592949, Apr. 20, 1994, Pieper, et al.
EP 596326, May, 11, 1994, Maier, et al.
DE 4241632, June 15, 1994, Himmelsbach, et al.
EP 0604800 A, Jul. 6, 1994, Himmelsbach, et al.
DE 4302051, (Der 94-235999/29) July, 28, 1994.
EP 0608858 A, Aug. 3, 1994, Linz, et al.
DE 4304650, (Der 94-256165/32), Aug, 18, 1994, Austel, et al.
EP 611660, Aug. 24, 1994, Austel, et al.
DE 4305388, (Der 94-264904/33), Aug.25, 1994, Himmelsbach, et al.
(P5D4005), EP 612741, (Der 94-265886/33), Aug. 31, 1994,
Himmelsbach, et al.
EP 0639575 A, Feb. 22, 1995, Linz, et al.
DE 4324580, Jan. 26, 1995, Linz, et al.
EP 0638553, Feb. 15, 1995, Himmelsbach, et al.
Hiummelsbach, et al., in XIIth Int. Symp. on Med. Chem. Basel,
Book of Abstracts, 47, 1992.
Austel, et al., Natl. Mtg. Amer. Chem. Soc. Book of Abstracts,
Denver, Div. Med. Chem., 1993.
Muller, et al., Orally Activity of BIBU 104, a Prodrug of the
Non-peptide Fibrinogen Receptor Antagonist BIBU 52, in Mice and
Monkeys, Thromb. Haem., 69, 975, 1993.
Univ. California
WO 94/14848, July, 7, 1994, Zanetti.
Univ. New York
WO 94/00144, June 29, 1993, Ojima, et at.
Yeda Res. and Dev. Co.
WO 93/09795, (Der 93-182236/22), Lido, et al.
Zeneca
WO 9422834, Oct. 13, 1994, Wayne, et al.
WO 9422835, Oct. 13, 1994, Wayne, et al.
EP 632016, Jan. 4, 1995, Brewster, et al.
EP 632019, Jan. 4, 1995, Brown, et al.
EP 632020, Jan. 4, 1995, Brown, et al.

In one particular embodiment, the fibrinogen receptor antagonist template A is the fused 6/7 ring bicyclic ring defined in Bondinell, et al., WO 93/00095, published Jan. 7, 1993, as defined by sub-formula (VI):

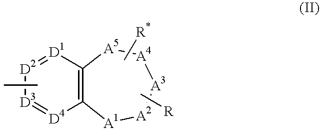

wherein

A¹ to A⁵ form an accessible substituted seven-membered ring, which may be saturated or unsaturated, optionally containing up to two heteroatoms chosen from the group of O, S and N wherein S and N may be optionally oxidized;

D¹ to D⁴ form an accessible substituted six membered ring, optionally containing up to two nitrogen atoms;

R is at least one substituent chosen from the group of R⁷, or Q—C₁₋₄alkyl, Q—C₂₋₄alkenyl, Q—C₂₋₄alkynyl, optionally substituted by one or more of ═O, R¹¹ or R⁷;

R* is H, Q—C₁₋₆alkyl, Q—C₁₋₆oxoalkyl, Q—C₂₋₆alkenyl, Q—C₃₋₄oxoalkenyl, Q—C₃₋₄oxoalkynyl, Q—C₂₋₄alkynyl, C₃₋₆cycloalkyl, Ar or Het, optionally substituted by one or more of R¹¹;

Q is H, C₃₋₆cycloalkyl, Het or Ar;

R⁷ is —COR⁸, —COCR′₂R⁹, —C(S)R⁸, —S(O)ₘOR′, —S(O)ₘNR′R″, —PO(OR′), —PO(OR′)₂, —B(OR′)₂, —NO₂ and Tet;

R⁸ is —OR′, —NR′R″, —NR′SO₂R′, —NR′OR′, —OCR′₂C(O)OR′, —OCR′₂OC(O)—R′, —OCR′₂C(O)NR′₂, CF₃ or AA¹;

R⁹ is —OR′, —CN, —S(O)ᵣR′, S(O)ₘNR′₂, —C(O)R′C(O)NR′₂ or —CO₂R′;

R¹¹ is H, halo, —OR¹², —CN, —NR′R¹², —NO₂, —CF₃, CF₃S(O)ᵣ, —CO₂R′, —CONR′₂, Q—C₀₋₆alkyl-, Q—C₁₋₆oxoalkyl-, Q—C₂₋₆alkenyl-, Q—C₂₋₆alkyl-, Q—C₀₋₆alkyloxy-, Q—C₀₋₆alkylamino- or Q—C₀₋₆alkyl—S(O)ᵣ—;

R¹² is R′, —C(O)R′, —C(O)NR′₂, —C(O)OR¹⁵, —S(O)ₘR′or S(O)ₘNR′₂;

R¹³ is R′, —CF₃, —SR′, or —OR′;

R¹⁴ is R′, C(O)R′, CN, NO₂, SO₂R′ or C(O)OR¹⁵;

R¹⁵ is H, C₁₋₆alkyl or Ar—C₀₋₄alkyl;

R′ is H, C₁₋₆alkyl, C₃₋₇cycloalkyl-C₀₋₄alkyl or Ar—C₀₋₄alkyl;

R″ is R′, —C(O)R′or —C(O)OR¹⁵;

R″′ is R″ or AA2;

AA1 is an amino acid attached through its amino group and having its carboxyl group optionally protected, and AA2 is an amino acid attached through its carboxyl group, and having its amino group optionally protected;

m is 1 or 2;

n is 0 to 3;

p is 0 or 1; and

t is 0 to 2; or

pharmaceutically acceptable salts thereof.

With reference to formula (II), suitably,

A¹ is CR¹R^{1′}, CR¹, NR¹, N, O or S(O)ₓ;

A² is CR²R^{2′}, CR², NR²;

A³ is CR³R^{3′}, CR³, NR³, N, O or S(O)ₓ;

A⁴ is CR⁴R^{4′}, CR⁴, NR⁴, or N;

A⁵ is CR⁵R^{5′}, CR⁵, NR⁵, N, O or S(O)ₓ;

D¹-D⁴ are CR¹¹, CR⁶ or N;

R¹ and R^{1′} are R* or R, or together are ═O;

R² and R^{2′} are R*, R or ═O;

R³ and R^{3′} are R*, R or ═O;

R⁴ and R^{4′} are R*, R or ═O;

R⁵ and R^{5′} are R*, R or ═O; and

x is 0 to 2.

More suitably, A¹ is CR¹R^{1′}, CR¹, NR¹, N, O or S; A² is CR²R^{2′}, NR² or CR²; A³ is CR³R^{3′}; A⁴ is CR⁴R^{4′}, CR⁴, NR⁴, or N; A⁵ is CR⁵R^{5′}, CR⁵, NR⁵, N, O; D¹-D⁴ are CH; R² or R⁴ are R; R³, R^{3′} and R⁵,R^{5′} are ═O or R*,H.

Preferably, A¹ is CHR¹, CR¹, NR″, N or S; A² is CR² or CR²R^{2′}; A³ is CR³R^{3′}; A⁴ is CR⁴R^{4′} or NR⁴; A⁵ is CR⁵R^{5′}, and D¹-D⁴ are CH.

In one embodiment, A¹ is CR¹, A² is CR², A³ is C═O, A⁴ is NR⁴ and A⁵ are CHR⁵.

In another embodiment, A¹ is NR¹, A² is CHCR², A³ is CR³R^{3′}, A⁴ is NR⁴, and A⁵ are C═O.

In yet another embodiment, A¹ and A⁴ are C═O, A² is NR², A³ is CHR^{3′} and A⁵ is NR⁵.

In a preferred embodiment, A¹ is NR¹, A² is CHR², A³ is C═O, A⁴ is NR′ and A⁵ is CHR⁵.

Representative sub-formulas of (II) are given by each of formulas (IIa)-(IIi) below:

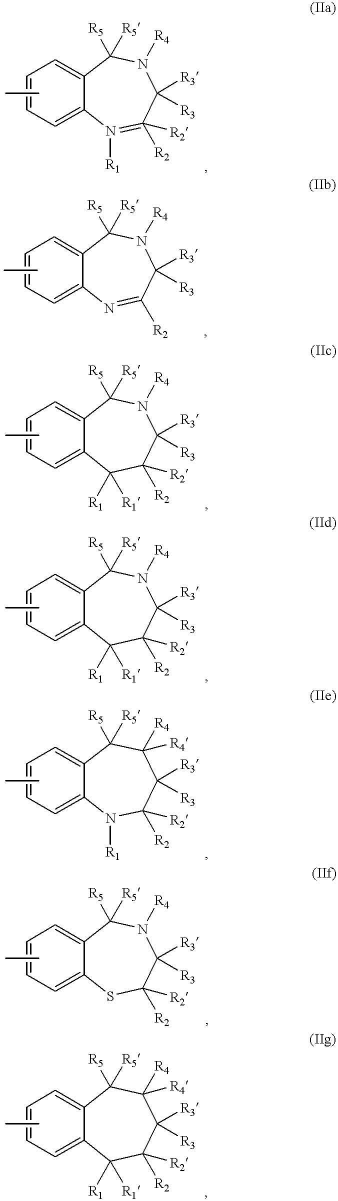

A preferred template is given by formula (III):

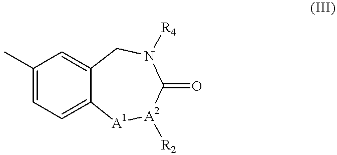

wherein

A¹-A² is NR¹—CH, NC(O)R³—CH, N═C, CR¹═C, CHR¹—CH, O—CH or S—CH;

R¹ is H, C₁₋₆ alkyl or benzyl;

R² is (CH₂)_{q}CO₂H;

R⁴ is H, C₁₋₆alkyl, Ar—C₀₋₆alkyl, Het-C₀₋₆alkyl, or C₃₋₆cycloalkyl-₀₋₆alkyl; and

q is 1, 2 or 3.

Preferably A¹-A² is NH—CH and R² is CH₂CO₂H. Suitably, R³ is methyl and W (as defined in formula (1)) is (CH₂)ₐNR′CO. Suitably Rⁱ is substituted by NHR′, CN, CO₂H, biotin, benzimidazole or optionally substituted phenyl.

Specific examples of vitronectin antagonists employing this template are:

(S)-7-[[[(6-Amino-2-pyridinyl)methyl]methylamino]carbonyl]-2,3,4,5-tetrahydromethyl-3-oxo-1H-1,4-benzodiazepine-2-acetic;

(S)-7-[[[(6-Amino-2-pyridinyl)methyl]amino]carbonyl]-2,3,4,5-tetrahydro-4-methyl-3-oxo-1H-1,4-benzodiazepine-2-acetic acid;

(S)-7-[[[(6-Ethylamino-2-pyridinyl)methyl]amino]carbonyl]-2,3,4,5-tetrahydro-4-methyl-3-oxo-1H-1,4-benzodiazepine-2-acetic acid; and

(±)-7-[[[(2-Amino-4-pyrimidinyl)methyl]methylamino]carbonyl]-2,3,4,5-tetrahydro-4-methyl-3-oxo-1H-1,4-benzodiazepine-2-acetic acid.

A preferred compound is (S)-7-[[[(6-Ethylamino-2-pyridinyl)methyl]amino]carbonyl]-2,3,4,5-tetrahydro-4-methyl-3-oxo-1H-1,4-benzodiazepine-2-acetic acid

Another embodiment of a benzodiazepine fibrinogen receptor template A is represented by the 1,4-benzodiazepine 2,5-dione of sub-formula (IV);

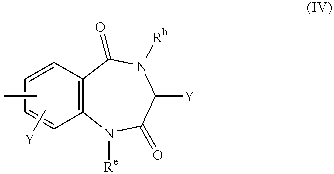

wherein:

Y is H, C₁₋₄alkyl, C₁₋₄alkoxy, C₁₋₄alkoxycarbonyl, F, Cl, Br, I, CF₃, OR^{f}, S(O)ₖR^{f}, COR^{f}, NO₂, N(R^{f})₂, CO(NR^{f})₂, CH₂N(R^{f})₂, methylenedioxy, CN, CO₂R^{f}, OC(O)R^{f}, or NHC(O)R^{f}; and

R^{h} is (CH₂)_{q}CO₂R^{f}.

Suitably R^{h} is CH₂CH₂CO₂H.

Entries (V)-(XV) in Table II summarize other illustrative fibrinogen receptor templates that are included within the scope of the present invention:

TABLE II
(V)

| | |
|---|---|
| 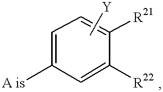 | 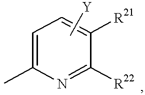 |
| 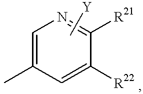 | 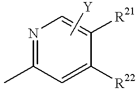 |
| or | 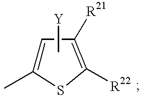 |

wherein:

R²¹ and R²² independently are H or —Z—CO₂R^{f} or Z—CON(R^{f})₂ with the proviso that one of R²¹ or R²² is —Z—CO₂R^{f} or Z—CON(R^{f})₂;

Z is —CH₂—, —O(CH₂)_{q}—, —NR^{f}(CH₂)_{q}—, —S(CH₂)_{q}, —CH₂CH₂—, —CH(CH₃)CH₂—, —(CH₂)₃—, —CH═CH—, —C(CH₃)═CH—, CH₂—CH═CH— or CH═CHCH₂; and

Y is H, C₁₋₄alkyl, C₁₋₄alkoxy, C₁₋₄alkoxycarbonyl, F, Cl, Br, I, CF₃, OR^{f}, S(O)ₖR^{f}, COR^{f}, NO₂, N(R^{f})₂, CO(NR^{f})₂, CH₂N(R^{f})₂, methylenedioxy or Z—COR^{f}, disclosed in Alig, et al., EP 0 381 033, published Aug. 8, 1990.

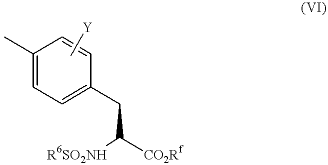

wherein:

R⁶ is aryl, C₁₋₁₀alkyl, C₃₋₆cycloalkyl, C₄₋₁₀aralkyl, C₁₋₁₀alkoxyalkyl, C₁₋₁₀alkaryl, C₁₋₁₀alkylthioalkyl, C₁₋₁₀alkoxythioalkyl, C₁₋₁₀alkylamino, C₄₋₁₀aralkylamino, C₁₋₁₀alkanoylamino, C₄₋₁₀aralkanoylamino, C₁₋₁₀alkanoyl, C₄₋₁₀aralkanoyl, or C₁₋₁₀carboxyalkyl; and

Y is H, C₁₋₄alkyl, C₁₋₄alkoxy, C₁₋₄alkoxycarbonyl, F, Cl, Br, I, CF₃, OR^{f}, S(O)ₖR^{f}, COR^{f}, NO₂, N(R^{f})₂, CO(NR^{f})₂, CH₂N(R^{f})₂, methylenedioxy, CN, CO₂R^{f}, OC(O)R^{f}, or NHC(O)R^{f},

disclosed in Egbertson, et al., EP 0 478 328, published Apr. 1, 1992.

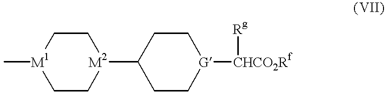

wherein:

M¹ is CH or N;

M² is CH or N, with the proviso that when M¹ is CH, M² is N; and

G′ is N or N⊕R″,

disclosed in Eldred, et al., EP 0542 363, published May 19, 1993.

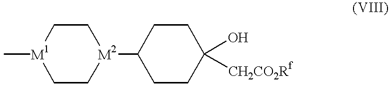

wherein:

M¹ is CH or N; and

M² is CH or N, with the proviso that when M¹ is CH, M² is N, disclosed in Porter, et al., EP 0 537 980, published Apr. 21, 1993.

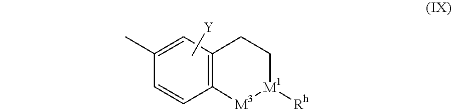

wherein:

M¹ is CH or N;

Y is H, C₁₋₄alkyl, C₁₋₄alkoxy, C₁₋₄alkoxycarbonyl, F, Cl, Br, I, CF₃, OR^{f}, S(O)ₖR^{f}, COR^{f}, NO₂, N(R^{f})₂, CO(NR^{f})₂, CH₂N(R^{f})₂, methylenedioxy, CN, CO₂R^{f}, OC(O)R^{f}, or NHC(O)R^{f};

D³ is CH₂ or C═O; and

R^{h} is (CH₂)_{q}CO₂R^{f},

disclosed in Klinnick, et al., EP 0 635,492, published Jan. 25, 1995.

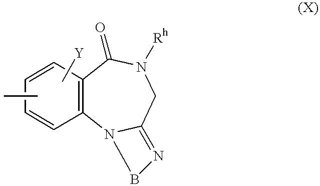

wherein:

Y is H, C₁₋₄alkyl, C₁₋₄alkoxy, C₁₋₄alkoxycarbonyl, F, Cl, Br, I, CF₃, OR^{f}, S(O)ₖR^{f}, COR^{f}, NO₂, N(R^{f})₂, CO(NR^{f})₂, CH₂N(R^{f})₂, methylenedioxy, CN, CO₂R^{f}, OC(O)R^{f}, or NHC(O)R^{f};

R^{h} is (CH₂)ₙCO₂R^{f}; and

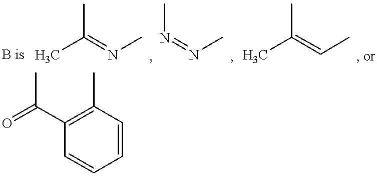

disclosed in Blackburn, et al., WO 95/04057, published Feb. 9, 1995.

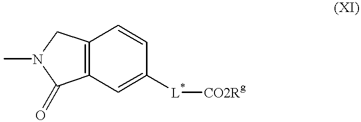

wherein:

L* is —C(O)NR^{g}—(CH₂)—, —C(O)—CH₂)_{q}—, NR^{g}—(CH₂)_{q}—, —O—(CH₂)_{q}—, or S(O)ₖ—(CH₂)_{q}—,

disclosed in Hartman, et al., EP 0 540 331, published May 5, 1993.

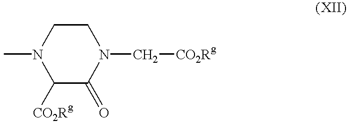

disclosed in Sugihara, et al., EP 0 529,858, published Mar. 3, 1993.

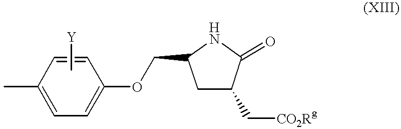

wherein:

Y is H, C₁₋₄alkyl, C₁₋₄alkoxy, C₁₋₄alkoxycarbonyl, F, Cl, Br, I, CF₃, OR^{f}, S(O)ₖR^{f}, COR^{f}, NO₂, N(R^{f})₂, CO(NR^{f})₂, CH₂N(R^{f})₂, methylenedioxy, CN, CO₂R^{f}, OC(O)R^{f}, or NHC(O)R^{f},

disclosed in Himmeisbach, et al., EP 0 483 667, published May 6, 1992.

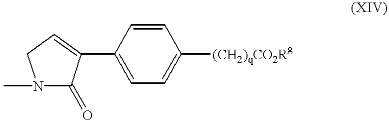

disclosed in Linz, et al., EP 0 567 968, published Nov. 3, 1993.

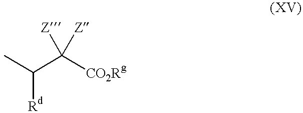

wherein:

R^{d} is Het-C₀₋₆alkyl; and

Z″, Z″′ independently are hydrogen, C₁₋₄alkyl, halo, OR^{f}, CN, S(O)ₖR^{f}, CO₂R^{f}, or OH,

disclosed in Bovy, et al., EP 0 539 343, published Apr. 28, 1993.

The above descriptions of fibrinogen receptor templates for use in the present invention were taken from pending published patent applications. Reference should be made to such patent applications for their full disclosures, including the variations possible for such templates and methods of preparing said templates, the entire disclosure of such patent applications being incorporated herein by reference.

In cases wherein the compounds of this invention may have one or more chiral centers, unless specified, this invention includes each unique nonracemic compound which may be synthesized and resolved by conventional techniques. In cases in which compounds have unsaturated carbon-carbon double bonds, both the cis (Z) and trans (E) isomers are within the scope of this invention. The meaning of any substituent at any one occurrence is independent of its meaning, or any other substituent's meaning, at any other occurrence.

Abbreviations and symbols commonly used in the peptide and chemical arts are used herein to describe the compounds of this invention. In general, the amino acid abbreviations follow the IUPAC-IUB Joint Commission on Biochemical Nomenclature as described in Eur. J. Biochem., 158, 9 (1984).

C₁₋₄alkyl as applied herein means an optionally substituted alkyl group of 1 to 4 carbon atoms, and includes methyl, ethyl, n-propyl, isopropyl, n-butyl, isobutyl and t-butyl. C₁₋₆alkyl additionally includes pentyl, n-pentyl, isopentyl, neopentyl and hexyl and the simple aliphatic isomers thereof. C₀₋₄alkyl and C₀₋₆alkyl additionally indicates that no alkyl group need be present (e.g., that a covalent bond is present).

Any C₁₋₄alkyl or C₁₋₆ alkyl, C₂₋₆ alkenyl, C₂₋₆ alkynyl or C₁₋₆ oxoalkyl may be optionally substituted with the group RX, which may be on any carbon atom that results in a stable structure and is available by conventional synthetic techniques. Suitable groups for R^{x} are C₁₋₄alkyl, OR′, SR′, C₁₋₄alkyl, C₁₋₄alkylsulfonyl, C₁₋₄alkylsulfoxyl, —CN, N(R′)₂, CH₂N(R′)₂, —NO₂, —CF₃, —CO₂R′, —CON(R′)₂, —CO R′, —N R′C(O) R′, OH, F, Cl, Br, I, N₃ or CF₃S(O)ᵣ—, wherein r is 0 to 2 and R′ is as defined with respect to formula (II).

Ar, or aryl, as applied herein, means phenyl or naphthyl, or phenyl or naphthyl substituted by one to three substituents, such as those defined above for alkyl, especially C₁₋₄alkyl, C₁₋₄alkoxy, C₁₋₄alkthio, CO₂H, N₃, trifluoroalkyl, OH, F, U, Br or I.

Het, or heterocycle, indicates an optionally substituted five or six membered monocyclic ring, or a nine or ten-membered bicyclic ring containing one to three heteroatoms chosen from the group of nitrogen, oxygen and sulfur, which are stable and available by conventional chemical synthesis. Illustrative heterocycles are benzofuryl, benzimidazole, benzopyran, benzothiophene, biotin, furan, imidazole, indoline, morpholine, piperidine, piperazine, pyrrole, pyrrolidine, tetrahydropyridine, pyridine, thiazole, thiophene, quinoline, isoquinoline, and tetra- and perhydro- quinoline and isoquinoline. Any accessible combination of up to three substituents on the Het ring, such as those defied above for alkyl that are available by chemical synthesis and are stable are within the scope of this invention.

C₃₋₇cycloalkyl refers to an optionally substituted carbocyclic system of three to seven carbon atoms, which may contain up to two unsaturated carbon-carbon bonds. Typical of C₃₋₇cycloalkyl are cyclopropyl, cyclobutyl, cyclopentyl, cyclopentenyl, cyclohexyl, cyclohexenyl and cycloheptyl. Any combination of up to three substituents, such as those defined above for alkyl, on the cycloalkyl ring that is available by conventional chemical synthesis and is stable, is within the scope of this invention.

The ring represented by

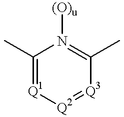

is a six-membered ring containing at least one nitrogen which is 2,6-disubstituted relative to such nitrogen. The ring may optionally have an additional nitrogen atom in the ring, and hence may be a pyrazine or a pyrimidine. The substituent R^{y} may be in any position on Q¹-Q³ which results in a stable structure. It will be apparent that when the value of u is 1 the compound described will be an N-oxide; whereas, when the value of u is 0 there is no oxygen substituent on the nitrogen. A pyridine ring is preferred.

Certain radical groups are abbreviated herein. t-Bu refers to the tertiary butyl radical, Boc refers to the t-butyloxycarbonyl radical, Fmoc refers to the fluorenylmethoxycarbonyl radical, Ph refers to the phenyl radical, Cbz refers to the benzyloxycarbonyl radical, BrZ refers to the o-bromobenzyloxycarbonyl radical, ClZ refers to the o-chlorobenzyloxycarbonyl radical, Bzl refers to the benzyl radical, 4-MBzl refers to the 4-methyl benzyl radical, Me refers to methyl, Et refers to ethyl, Ac refers to acetyl, Alk refers to C₁₋₄alkyl, Nph refers to 1- or 2-naphthyl and cHex refers to cyclohexyl. Tet refers to 5-tetrazolyl.

Certain reagents are abbreviated herein. DCC refers to dicyclohexylcarbodiimide, DMAP refers to dimethylaminopyridine, DIEA refers to diisopropylethyl amine, EDC refers to 1-(3-dimethylaminopropyl)-3-ethylcarbodiimide, hydrochloride. HOBt refers to 1-hydroxybenzotriazole, THF refers to tetrahydrofuran, DIEA refers to diisopropylethylamine, DME refers to dimethoxyethane, DMF refers to dimethylformamide, NBS refers to N-bromosuccinimide, Pd/C refers to a palladium on carbon catalyst, PPA refers to 1-propanephosphonic acid cyclic anhydride, DPPA refers to diphenylphosphoryl azide, BOP refers to benzotriazol-1-yloxy-tris(dimethylamino)phosphonium hexafluorophosphate, HF refers to hydrofluoric acid, TEA refers to triethylamine, TFA refers to trifluoroacetic acid, PCC refers to pyridinium chlorochromate.

Compounds of the formula (I) are generally prepared by reacting a compound of formula (XVI) with a compound of formula (XVII), wherein L¹ and L² are groups which may react to form a covalent bond in the moiety W, by methods generally known in the art.

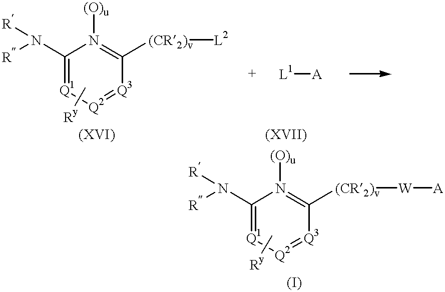

Typical methods include coupling to form amide bonds, nucleophilic displacement reactions and palladium catalyzed couplings.

For instance, when W contains an ether or amine linkage, the bond may be formed by a displacement reaction, and one of L¹ and L² will contain an amino or hydroxy group and the other will contain a displaceable group, such as a chloro, bromo or iodo group. When W contains an amide bond, typically one of L¹ and L² will contain an amino group, and the other will contain a carboxylic acid group. In another approach, L¹ may be an aryl or heteroaryl bromide, iodide or trifluoromethylsulfonyloxy derivative and L² may contain an amino group and the amide linkage may be formed by palladium-catalyzed aminocarbonylation with carbon monoxide in a suitable solvent such as dimethylformamide or toluene.

It will be apparent that the precise identity of L¹ and L² will be dependent upon the site of the linkage being formed. General methods for preparing the linkage —(CHR″)ᵣ—U—(CHR″)ᵣ—V— are described, for example, in EP-A 0 372 486 and EP-A 0 381 033 and EP-A 0 478 363, which are incorporated herein by reference.

For instance, if V is CONH, L¹ may be —NH₂, L² may be OH (as in an acid) or Cl (as in an acid chloride). For instance, (2-amino-pyrid-6-yl)(CH₂)ₐ—COCl may be reacted with a suitable amine. When L² is OH, a coupling agent is used.

Similarly, if V is NHCO, L¹ may be —CO₂H or CO—Cl, L² may be —NH₂. For instance, (2-amino-pyrid-6-yl)(CH₂)ₐ—NHR′ may be reacted with a suitable carboxyolic acid.

Where V is NHSO₂, L¹ may be SO₂Cl, L² may be —NH₂ as above. Where V is SO₂NH, L¹ may be —NH₂ and L² may be SO₂Cl. Methods to prepare such sulfonyl chlorides are disclosed, for instance, in J. Org. Chem., 23, 1257 (1958).

If V is CH═CH, L¹ may be —CHO, L² may be CH═P—Ph₃. Alternately, L¹ may be CH═P—Ph₃ and L² may be CHO. For instance, (2-amino-pyrid-6-yl)(CH₂)ₐ—CHO may be reacted with a suitable phosphorane.

Where V is CH₂CH₂ may be obtained by reduction of a suitably protected compound wherein V is CH═CH.

Where V is CH₂O, CH₂N or C≡C, L¹ may be —OH, —NH or —C≡CH, respectively; L² may be —Br or —I. Similarly where U or V is OCH₂, NR′CH₂ or C≡C, L¹ may be —CH₂Br and L² may be —OH, —NH or —C≡C H, respectively. For example, (2-amino-pyrid-6-yl)(CH₂)ₐ—Br may be reacted with an appropriate amine, alkoxide or acetylene. Alternately, when U or V is C≡C, L¹ may be Br, I or CF₃SO₃, L² may be C≡CH and the coupling may be catalyzed by palladium and a base.

Compounds wherein V is CHOHCH₂ may be prepared from a suitably protected compound where V is CH═CH by the procedure disclosed in J. Org. Chem., 54, 1354 (1989).

Compounds wherein V is CH₂CHOH may be obtained from a suitably protected compound where V is CH═CH by hydroboration and basic oxidation as disclosed in Tet. Lett., 31, 231 (1990).

The core 6-7 fused ring fibrinogen template of formula (II) is prepared by methods well known in the art, e.g., Hynes, et al., J. Het. Chem., 1988, 25, 1173; Muller, et al., Helv. Chim. Acta., 1982, 65, 2118; Mori, et al., Heterocycles, 1981, 16, 1491. Similarly, methods for preparing benzazepines, 1,4-benzothiazepines, 1,4-benzoxazepines and 1,4-benzodiazepines are known and are disclosed, for instance, in Bondinell, et al., International Patent Application WO 93/00095.

Representative fibrinogen antagonist templates may be prepared according to Schemes A-AA, which follow:

Scheme A describes a method of exemplary fibrinogen receptor templates described in Blackburn, et. al., WO 93/08174.

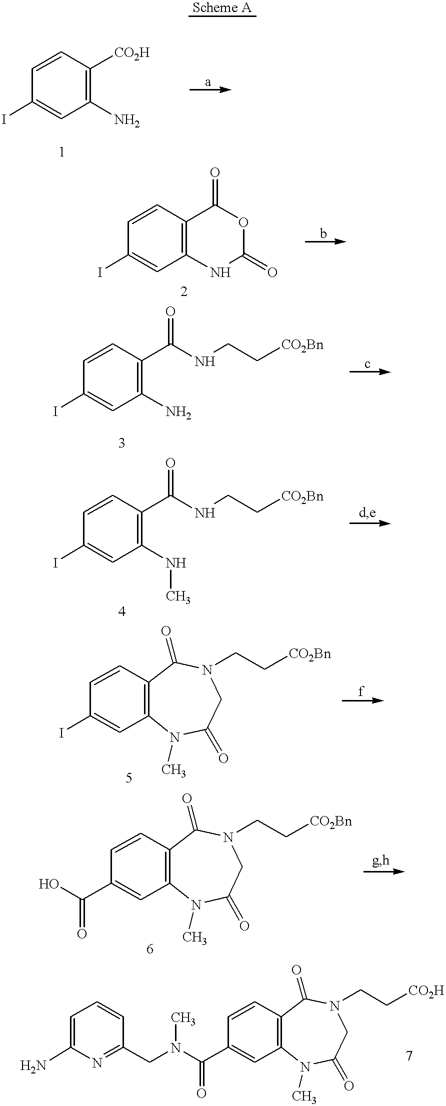

a) COCl₂, Na₂CO₃, toluene; b) β-alanine benzyl ester tosylate, DMAP, pyridine; c) CH₃I, 2,6-lutidine, DMF; d) α-bromoacetyl bromide, Et₃N, CH₂Cl₂; e) NaH, DMF, f) Pd(OAc)₂, dppf, CO, DMSO, 65° C., 18 h; g) 6-(methylamino)methyl-2-pyridinamine, EDC, HOBT.H₂O, DIEA, CH₃CN; h) H₂, 10% Pd/C, EtOH.

Scheme B describes a method of preparing exemplary fibrinogen receptor templates described in Blackburn, et. al., WO 95/04057.

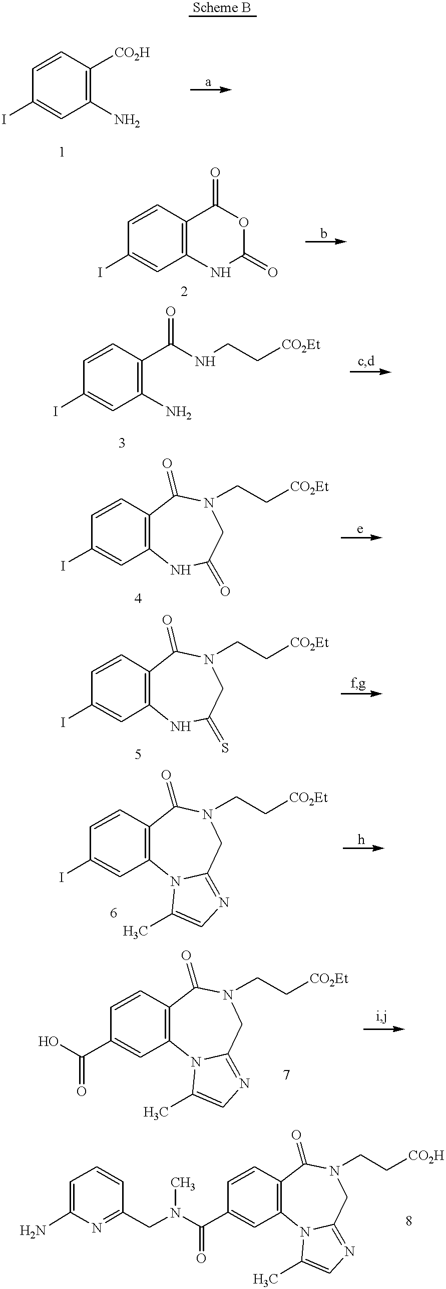

a) COCl₂, NaHCO₃, toluene; b) β-alanine ethyl ester hydrochloride, DMAP, pyridine; c) α-bromoacetyl bromide, Et₃N, CH₂Cl₂; d) NaH, DMF; e) Lawesson's reagent, THF, 50° C., 2 h; f) CH₃I, NaOH, (n-Bu)4N.HSO₄, CH₂Cl₂, H₂O, RT, 2 h; g) propargyl amine, toluene, pyridine hydrochloride, reflux, 6 h; h) Pd(OAc)₂, dppf, CO, DMSO, 65° C., 18 h; i) 6-(methylamino)methyl-2-pyridinamine, EDC, HOBT.H₂O, DIEA, CH₃CN; j) LiOH, H₂O, THF, 18 h.

Scheme C describes a method of preparing exemplary fibrinogen receptor templates described in Porter, et al., EP 0542363.

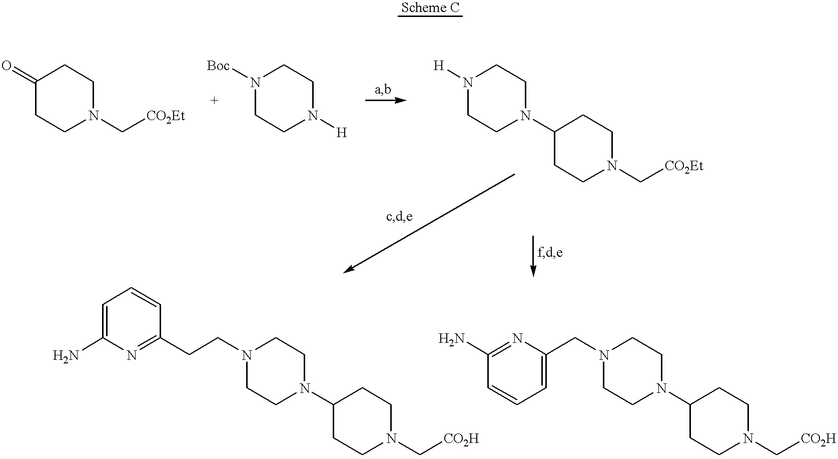

a) NaBH₃CN, HCl, CH₃OH; b) HCl, dioxaine, CH₂Cl₂; c) 6-methyl-2-(phthalimido)pyridine, H₂CO, EtOH; d) NaOH, H₂O, CH₃OH; d) hydrazine hydrate, EtOH, reflux; f) 6-bromomethyl-2-(phthalamido)pyridine, NaHCO_{3,} CH₃CN.

Scheme D describes a method of preparing exemplary fibrinogen receptor templates described in Porter, et al., EP 0537980.

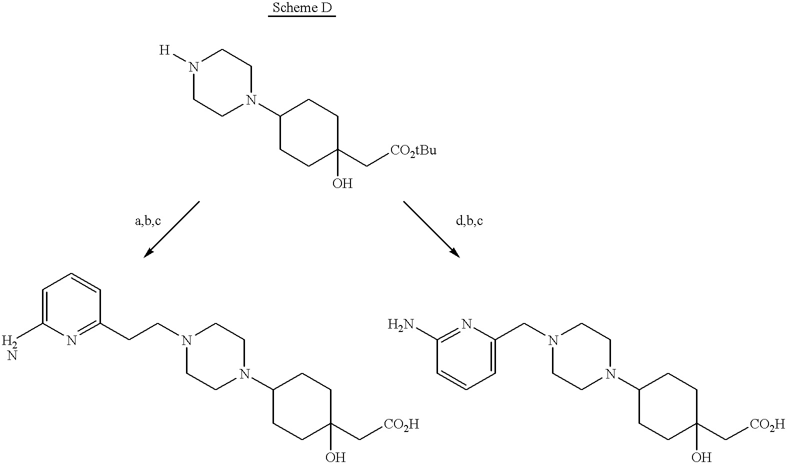

a) 6-methyl-2-(phthalimido)pyridine, H₂CO, EtOH; b) NaOH, H₂O, CH₃OH; c) hydrazine hydrate, EtOH, reflux; d) 6-bromomethyl-2-(phthalimido)pyridine, NaHCO₃, CH₃CN.

Scheme E describes a method of preparing exemplary fibrinogen receptor templates described in Beavers, et al., WO 95/25091.

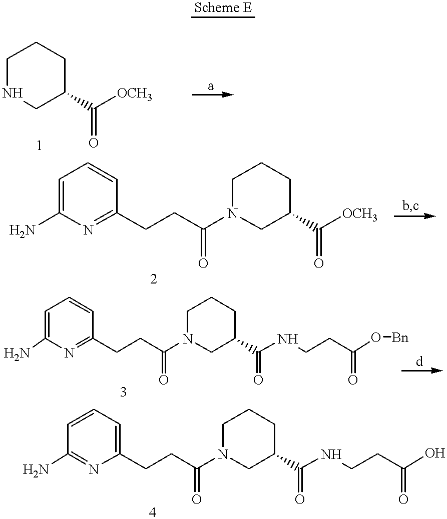

a) (6-amino-2-pyridinyl)propionic acid, BOP—Cl, NMM, CH₂Cl₂; b) LiOH, H₂O, THF; c) β-alanine benzyl ester, EDC, HOBT, NMM, CH₂Cl₂; d) H₂, 10% Pd/C, AcOH, THF, H₂O.

Following the procedures of Beavers, et al., WO 95/25091, Example 1, except substituting 3-(6-amino-2-pyridinyl)propionic acid, Bondinell, et al., WO 95/25091, for N^{α}-Boc-D-lys(Cbz)—OH, gives E4.

Scheme F describes a method of preparing exemplary fibrinogen receptor templates described in Hartman, et al., EP 0540334.

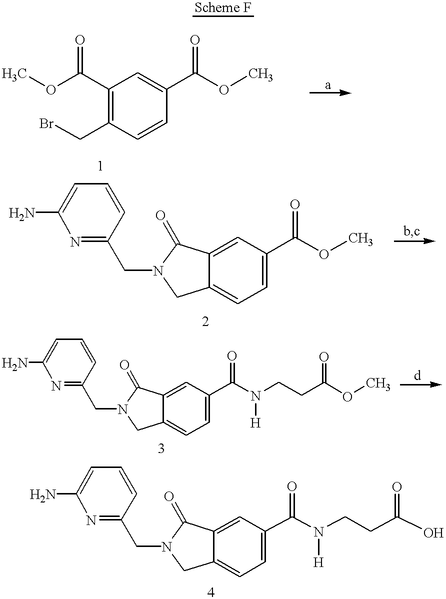

a) 6-aminomethyl-2-pyridinamine, Et₃N, benzene; b) 1.0N LiOH, H₂O, CH₃OH; c) α-alanine ethyl ester, BOP, Et₃N, CH₃CN; d) LiOH, H₂O, TBF, CH₃OH.

Dimethyl 4-(bromomethyl)benzene-1,3-dicarboxylate, F-1, is treated with a suitably functionalized amine, such 6-aminomethyl-2-pyridinamine, under the general conditions described for 2,3-dihydro-N-(2-carboxyethyl)-2-[2-(piperidinyl)ethyl]-3-oxo-1H-isoindole-5-carboxamide in Hartman, et al., EP 0540334, to give F-4.

Scheme G describes a method of preparing exemplary fibrinogen receptor templates described in Egbertson, et al., EP 0478363.

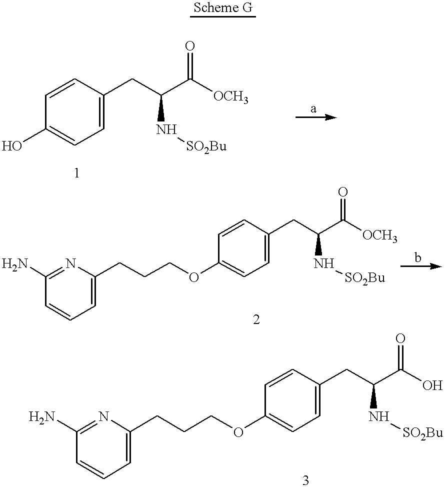

a) 3-(6-amino-2-pyridinyl)propanol, Ph₃P, DEAD, CH₂Cl₂, benzene; b) 1.0N LiOH, THF, H₂O.

N-(n-Butylsulfonyl)-L-tyrosine methyl ester, G-1, is treated with a suitably functionalized alcohol, such as 3-(6-amino-2-pyridinyl)propanol, prepared following the procedures of Warter, et al., Org. Synth. 1943, 23, 83, and Bruekelman, et al., J. Chem. Soc. Perkin Trans. I, 1984, 2801-2807, to give G-3.

Scheme H describes a method of preparing exemplary fibrinogen receptor templates described in Duggan, et al., J. Med. Chem. 1995, 38, 3332.

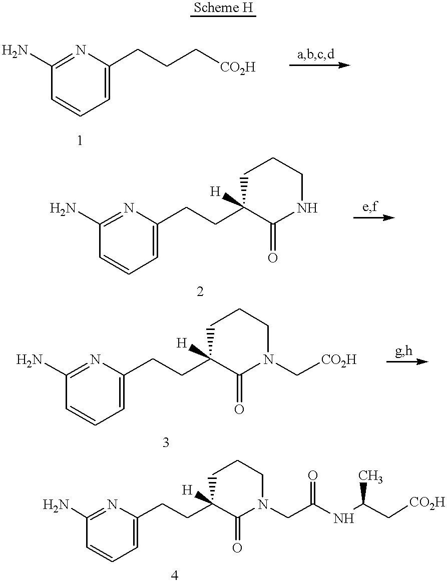

a) pivaloyl chloride, Et₃N, THF, (S)-benzyl-2-oxazolidinone; b) Ti(O—i—Pr)Cl₂, acrylonitrile, DIEA, CH₂Cl₂; c) H₂, PtO₂, CH₃OH, CHCl₃; d) NaHCO₃, CH₃CN; e) NaHMDS, ethyl bromoacetate; f) 1N NaOH, CH₃OH; g) 3(R)-methyl-β-alanine ethyl ester HCl, EDC, HOBT, Et₃N, DMF; h) 1N NaOH, CH₃OH.

A suitably functionalized carboxylic acid, such as 4-(6amino-2-pyridinyl)butanoic acid, H-1, is activated and reacted with a chiral auxiliary such as lithium (S)-4-benzyl-2-oxazolidinone to form a chiral Evans reagent. Alkylation of the titanium enolate with acrylonitrile, followed by nitrile reduction and lactam formation affords lactam H-2. Alkylation of the lactam with agents such as ethyl bromoacetate followed by ester saponification yields the carboxylic acid H-3. The resulting carboxylic acid derivative H-3 is converted to an activated form of the carboxylic acid using, for example, EDC and HOBt, or SOCl₂, and the activated form is subsequently reacted with an appropriate amine, for instance the 3(R)-methyl-β-alanine ethyl ester, in a suitable solvent such as DMF, CH₂Cl₂, or CH₃CN. Depending on whether acid neutralization is required, an added base, such as DIEA or pyridine, may be used. Many additional methods for converting a carboxylic acid to an amide are known, and can be found in standard reference books, such as “Compendium of Organic Synthetic Methods”, Vol. I-VI (published by Wiley-Interscience), or Bodansky, “The Practice of Peptide Synthesis” (published by Springer-Verlag). Hydrolysis of the ethyl ester is accomplished according to the general conditions described for the conversion of H-2 to H-3, to provide the carboxylic acid H4. Alternatively, the intermediate carboxylate salt of can be isolated, if desired, or a carboxylate salt of the free carboxylic acid can be prepared by methods well-known to those of skill in the art.

Scheme I describes a method of preparing exemplary fibrinogen receptor templates described in WO 93/07867.

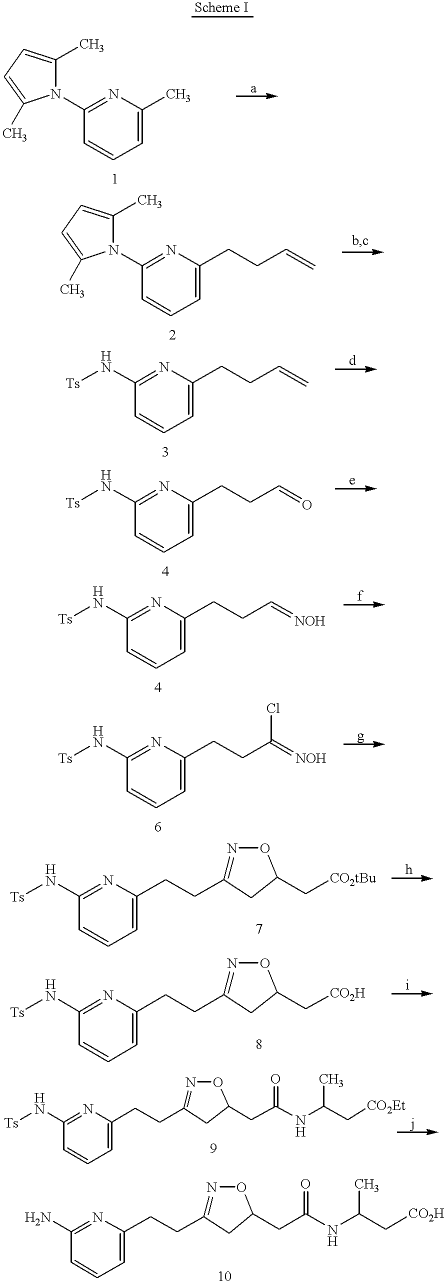

a) LDA, THF, alkyl bromide; b) NH₂OH.HCl, EtOH, H₂O; c) TsCl, NaH, THF; d) O₃, CH₂Cl₂, CH₃OH, DMS; e) NH₂OH.HCl, NaOAc, CH₃OH; f) NCS, DMF; g) tert-butyl 3-butenoate, Et₃N; h) 4M HCl, dioxane, CH₂Cl₂; i) ethyl 3-aminobutyrate, EDC, HOBt-H₂O, DIEA, CH₃CN; j) 1.0N LiOH, THF, H₂O.

The readily available aminopyridine derivative I-1, J. Chem. Soc. Perkin Trans. I 1984, 2801, is converted to the alkylated derivative I-2 by the general protocol described by Meakins, J. Chem. Soc. Perkin Trans. I 1984, 2801. Thus, I-1 is deprotonated with an amide base, such as lithium diisopropylamide or lithium bis(trimethylsilyl)amide, and the resulting metalated species is alkylated with an appropriate alkylating agent, for instance allyl bromide, to afford the butenyl derivative I-2. Generally, THF or ethylene glycol dimethyl ether are the solvents of choice for an alkylation reaction, although THF in the presence of various additives, for instance HMPA or TMEDA, can be used. The 2,5-dimethylpyrrole protecting group is conveniently removed at this stage using the general protocol described by Meakins (see reference above). Thus, I-2 is reacted with hydroxylamine hydrochloride in an appropriate solvent, e.g., aqueous EtOH, to afford the corresponding deprotected aminopyridine. Protection of the amino group of this aminopyridine can be accomplished by reaction with a sulfonyl chloride, for instance p-toluenesulfonyl chloride, in the presence of a suitable base, generally NaH or an aqueous alkali metal hydroxide, in an inert solvent, preferably THF, to afford I-3. Alternative protecting groups known to those of skill in the art may be used, as long as they are compatible with the subsequent chemistry and can be removed when desired. Such protecting groups are described in Greene, “Protective Groups in Organic Synthesis” (published by Wiley-Interscience). Oxidative cleavage of the olefin of I-3 to afford the aldehyde I-4 can be conveniently accomplished by ozonolysis in an inert solvent, usually CH₂Cl₂ or a mixture of CH₂Cl₂ and CH₃OH, followed by in-situ reduction of the ozonide with a suitable reducing agent, generally dimethylsulfide (DMS) or triphenylphosphine. Alternative methods for oxidative cleavage, such as the Lemieux-Johnson reaction, J. Org. Chem. 1956, 21, 478, can also be used. The aldehyde is converted to the aldoxime I-5 by standard procedures known to those of skill in the art, and this aldoxime is oxidized to the oximinoyl chloride derivative I-6 by the methods described in WO 95/14682 and WO 95/14683. Reaction of I-6 with an olefin, such as tert-butyl 3-butenoate, Tet. Lett. 1985, 26, 381-384, in the presence of a suitable base, for instance Et₃N or DIEA, in an inert solvent such as benzene or toluene, according to the protocol described in WO 95/14682 and WO 95/14683, gives the cycloadduct I-7. The tert-butyl ester of I-7 is removed under standard acidic conditions, generally TFA in CH₂Cl₂ or HCl in dioxane, to give the carboxylic acid I-8. The carboxylic acid is activated using, for example, EDC and HOBt, or SOCl₂, and the activated form is subsequently reacted with an appropriate amine, for instance a suitable derivative of β-alanine, in a neutral solvent, such as DMF, CH₂Cl₂, or CH₃CN, to afford I-9. Depending on whether acid neutralization is required, an added base, such as DIEA or pyridine, may be used. Many additional methods for converting a carboxylic acid to an amide are known, and can be found in standard reference books, such as “Compendium of Organic Synthetic Methods”, Vol. I-VI (published by Wiley-Interscience), or Bodansky, “The Practice of Peptide Synthesis” (published by Springer-Verlag). Derivatives of β-alanine are readily available in either racemic or optically pure form by a variety of methods known to those of skill in the art. A representative method is described in WO 93/07867. The ethyl ester and sulfonyl protecting groups of I-9 are removed using aqueous base, for example, LiOH in aqueous THF or NaOH in aqueous CH₃OH or EtOH. The intermediate carboxylate salt is acidified with a suitable acid, for instance TFA or HCl, to afford the carboxylic acid I-10. Alternatively, the intermediate carboxylate salt can be isolated, if desired, or a carboxylate salt of the free carboxylic acid can be prepared by methods well-known to those of skill in the art.

Scheme J describes a method of preparing exemplary fibrinogen receptor templates described in Alig, et al., EP 0372486.

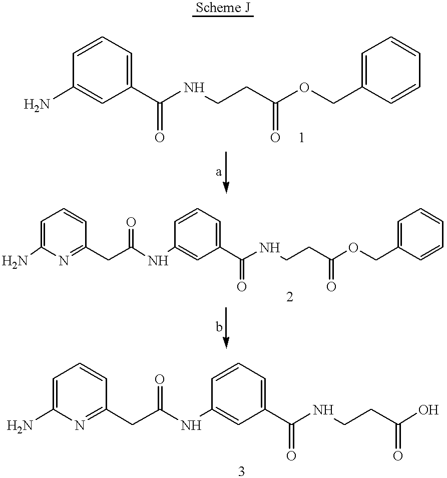

a) (6amino-2-pyridinyl)acetic acid, EDC, DIEA, DMF; b) NaOH, H₂O, CH₃OH.

J-1, prepared as described in Alig et al., EP 0372486, is condensed with a suitable substituted carboxylic acid, such as (6-amino-2-pyridinyl)acetic acid, prepared by saponification of ethyl (6amino-2-pyridinyl)acetate, Awaya, et al., Chem. Pharm. Bull. 1974, 22, 1414, in the presence of EDC and DIEA, and in a suitable solvent, e.g., DMF or CH₃CN, to give J-2. Many additional methods for converting a carboxylic acid to an amide are known, and can be found in standard reference books, such a “Compendium of Organic Synthesis”, Vol. I-VI (published by Springer-Verlag). Hydrolysis of the ester in J-2 is accomplished by saponification with a suitable reagent, e.g., NaOH, in a suitable solvent, e.g., aqueous methanol. Alternatively, the benzyl ester in J-2 may be converted to the acid by treatment with hydrogen and a suitable catalyst, e.g., Pd/C, in a suitable solvent, e.g., CH₃OH, EtOH, or AcOH.

Scheme K describes a method of preparing exemplary fibrinogen receptor templates described in Alig, et al., EP 0505868.

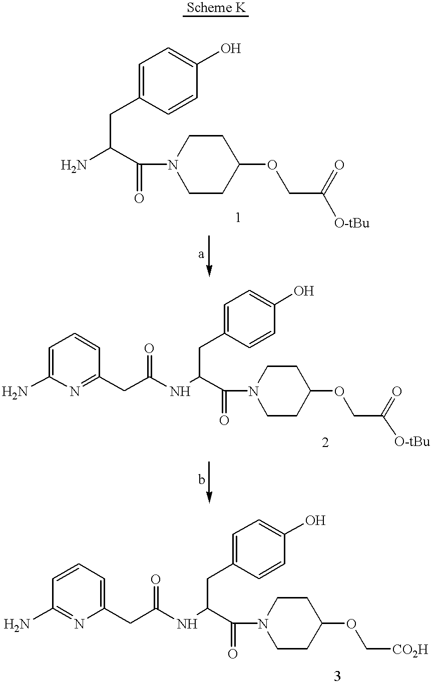

a) (6-amino-2-pyridinyl)acetic acid, EDC, DIEA, DMF; b) CF₃CO₂H, CH₂Cl₂.

K-1, prepared as described in Alig et al., EP 0505868, is condensed with a suitable substituted carboxylic acid, such (6-amino-2-pyridinyl)acetic acid, prepared by saponification of ethyl (6amino-2-pyridinyl)acetate, Awaya, et al., Chem. Pharm. Bull. 1974, 22, 1414, in the presence of EDC and DIEA, in a suitable solvent, e.g., DMF or CH₃CN, to give K-2. Many additional methods for converting a carboxylic acid to an amide are known, and can be found in standard reference books, such as “Compendium of Organic Synthesis”, Vol. I-VI (published by Springer-Verlag). Hydrolysis of the ester in J-2 is accomplished with trifluoroacetic acid or hydrogen chloride to give K-3. Alternatively, the ester in K-2 may be saponified with a suitable reagent, e.g., 1N NaOH, in a suitable solvent, e.g., CH₃OH.

Scheme L describes a method of preparing exemplary fibrinogen receptor templates described in WO 93/07867.

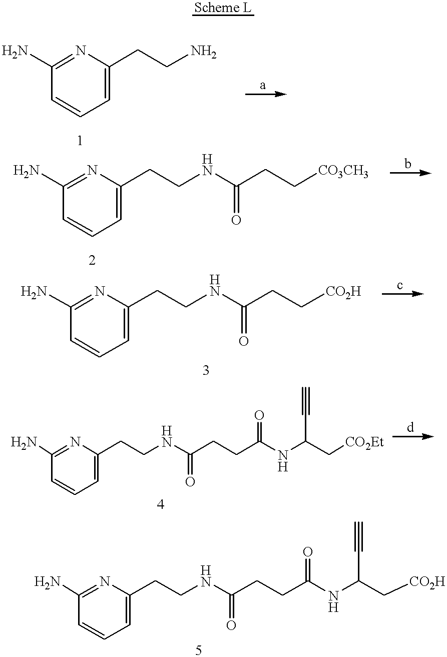

a) 3-(carbomethoxy)propionyl chloride, DIEA, CH₂Cl₂; b) 1.0N NaOH, CH₃OH; c) ethyl 3-amino-4-pentynoate, EDC, HOBt.H₂O, DIEA, CH₃CN; d) 1.0N LiOH, THF, H₂O.

A suitably functionalized amine, such as 2-amino-6-(2-aminomethyl)pyridine, prepared following the procedures of Preparation 13 in Bondinell, et al., WO 93/00095, for the preparation of 2-aminopyridine-4-ethanamine dihydrochloride, except substituting 2-amino-6-picoline for the 2-amino-4-picoline is reacted with 3-(carbomethoxy)propionyl chloride in the presence of an appropriate acid scavenger, such as Et₃N, DIEA, or pyridine, in a neutral solvent, generally CH₂Cl₂, to afford L-2. The methyl ester of L-2 is hydrolyzed using aqueous base, for example, LiOH in aqueous THF or NaOH in aqueous CH₃OH or EtOH, and the intermediate carboxylate salt is acidified with a suitable acid, for instance TFA or HCl, to afford the carboxylic acid L-3. Alternatively, L-1 can be reacted with succinic anhydride in the presence of an appropriate base, such as Et₃N, DIEA, or pyridine, in a neutral solvent, generally CH₂Cl₂, to afford L-3 directly. The resulting carboxylic acid derivative L-3 is converted to an activated form of the carboxylic acid using, for example, EDC and HOBt, or SOCl₂, and the activated form is subsequently reacted with an appropriate amine, for instance the known ethyl 3-amino4-pentynoate (WO 93/07867), in a suitable solvent such as DMF, CH₂Cl₂, or CH₃CN, to L-4. Depending on whether acid neutralization is required, an added base, such as DIEA or pyridine, may be used. Many additional methods for converting a carboxylic acid to an amide are known, and can be found in standard reference books, such as “Compendium of Organic Synthetic Methods”, Vol. I-VI (published by Wiley-Interscience), or Bodansky, “The Practice of Peptide Synthesis” (published by Springer-Verlag). Hydrolysis of the ethyl ester of L-4 is accomplished according to the general conditions described for the conversion of L-2 to L-3, to provide the carboxylic acid L-5. Alternatively, the intermediate carboxylate salt of can be isolated, if desired, or a carboxylate salt of the free carboxylic acid can be prepared by methods well-known to those of skill in the art.

Scheme M describes a method of preparing exemplary fibrinogen receptor templates described in Sugihara, et al., EP 0529858.

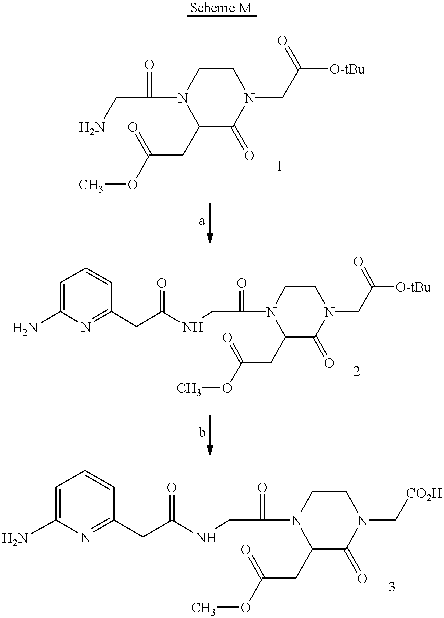

a) (6amino-2-pyridinyl)acetic acid, EDC, DIEA, DMF; b) CF₃CO₂H, CH₂Cl₂.

M-1, prepared as described in Sugihara, et al., EP 0529858, is condensed with a suitable substituted carboxylic acid, such as (6-amino-2-pyridinyl)acetic acid, prepared by saponification of ethyl (6-amino-2-pyridinyl)acetate, Awaya, et al., Chem. Pharm. Bull. 1974, 22, 1414, to give M-2, and the tert-butyl ester is cleaved with TFA, following the general procedure of Sugihara, et al., Example 59, to give M-3. Many additional methods for converting a carboxylic acid to an amide are known, and can be found in standard reference books, such as “Compendium of Organic Synthesis”, Vol. I-VI (published by Springer-Verlag).

Scheme N describes a method of preparing exemplary fibrinogen receptor templates described in Himmelsbach, et. al., AU-A-86926/91.

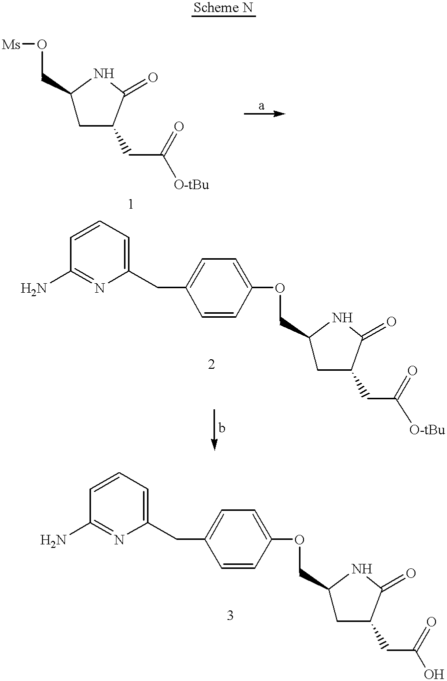

a) 4-[(6-amino-2-pyridinyl)methyl]phenol, Cs₂CO₃, DMF; b) 1N NaOH, CH₃OH.

Compound N-1, prepared as described by Himmelsbach, et al., AU-A-86926/91, Example VI(28), is treated with a suitable substituted phenol, such as 4-[(6-amino-2-pyridinyl)methyl]phenol, prepared from the corresponding anisole, Ife, et al., WO 9426715, with hydrobromic acid, following the general method of Himmelsbach et al., Example 3(51), to give N-2. The tert-butyl ester in N-2 is hydrolyzed with 1N NaOH in CH₃0H to give N-3. Alternatively, the tert-butyl ester may be cleaved with TFA or HCl in a suitable solvent such as CH₂Cl₂.

Scheme O describes a method of preparing exemplary fibrinogen receptor templates described in Linz, et al., EP 0567968.

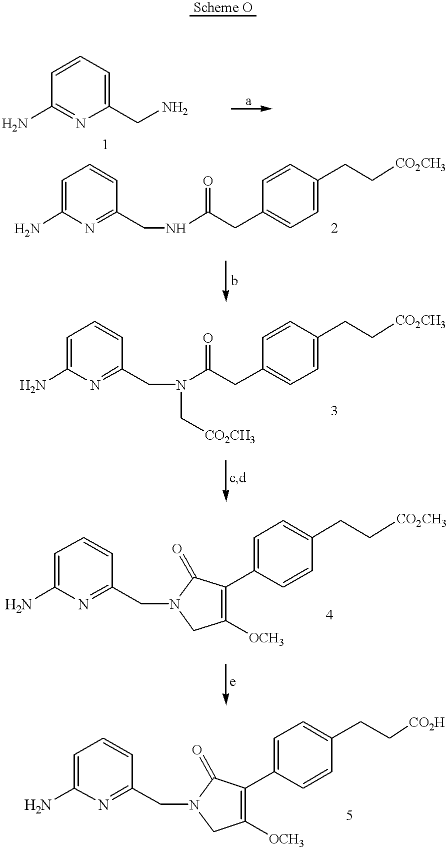

a) 6-aminomethyl-2-pyridinamine, Ph2POCl, Et₃N, DMAP, THF; b) NaH, BrCH₂CO₂CH₃, DMF; c) KOtBu, CH₃I, DMF; e) LiOH, H₂O, TBF.

Following the procedures of Linz, et al., EP 0567968, except substituting 6-amino-2-pyridinyl)methylamine for 4-cyanoaniline, gives O-5.

Scheme P describes a method of preparing exemplary fibrinogen receptor templates described in Wayne, et al., WO 94/22834.

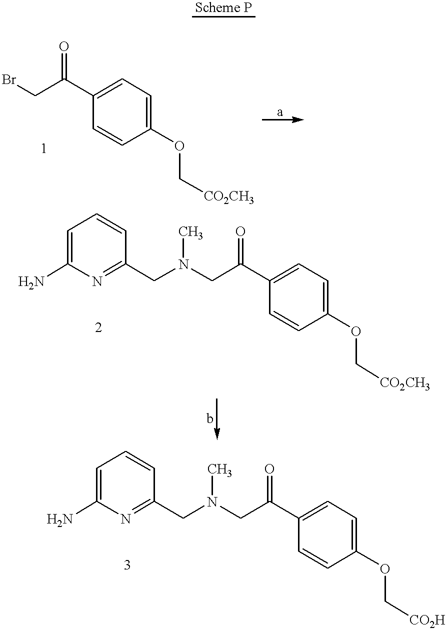

a) 6-methylamino)methyl-2-pyridinamine, CH₃CN; b) 1N NaOH, CH₃OH

Following the procedures of Wayne, et al., WO 94/22834, Example 1-2, except substituting 6-(methylamino)methyl-2-pyridinamine for 1-(4-pyridyl)piperazine gives P-3.

Scheme Q describes a method of preparing exemplary fibrinogen receptor templates described in Wayne, et al., WO 94/22834.

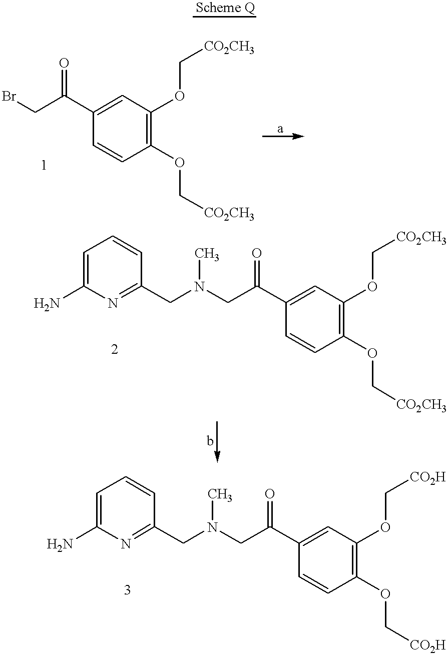

a) 6-(methylamino)methyl-2-pyridinamine, CH₃CN; b) 1N NaOH, CH₃OH

Following the procedures of Wayne, et al., WO 94/22834, Example 3-4, except substituting 6-(methylamino)methyl-2-pyridinamine for 1-(4-pyridyl)piperazine gives Q-3.

Scheme R describes a method of preparing exemplary fibrinogen receptor templates described in Alig, et al., EP 0381033.

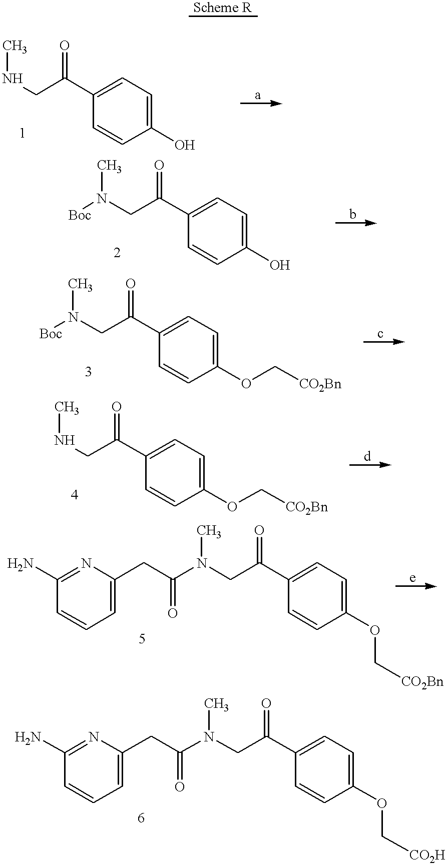

a) (Boc)₂O, NaOH, dioxane, H₂O; b) BrCH₂CO₂Bn, K₂CO₃, acetone; c) 4M HCl, dioxane; d) (6-amino-2-pyridinyl)acetic acid, EDC, DIEA, DMF; e) 1N NaOH, CH₃OH.

R-1 is treated with di-tert-butyl dicarbonate and sodium hydroxide in aqueous dioxane to afford R-2, which is alkylated on the phenolic oxygen with benzyl bromoacetate and potassium carbonate in acetone to give R-3. The Boc group in R-3 is removed with hydrogen chloride in dioxane, and the resulting R4 is acylated on nitrogen with (6-amino-2-pyridinyl)acetic acid, prepared by saponification of ethyl (6-amino-2-pyridinyl)acetate, Awaya, et al., Chem Pharm. Bull. 1974, 22, 1414, EDC and DIEA in DMF to give R-5. The benzyl ester in R-5 is saponified to give R-6. Alternatively, the benzyl ester may be cleaved by treatment with H₂ and a suitable catalyst, such as Pd/C, in a suitable solvent, such as CH₃OH, EtOH, or AcOH.

Scheme S describes a method of preparing exemplary fibrinogen receptor templates described in Alig, et al., EP 0381033.

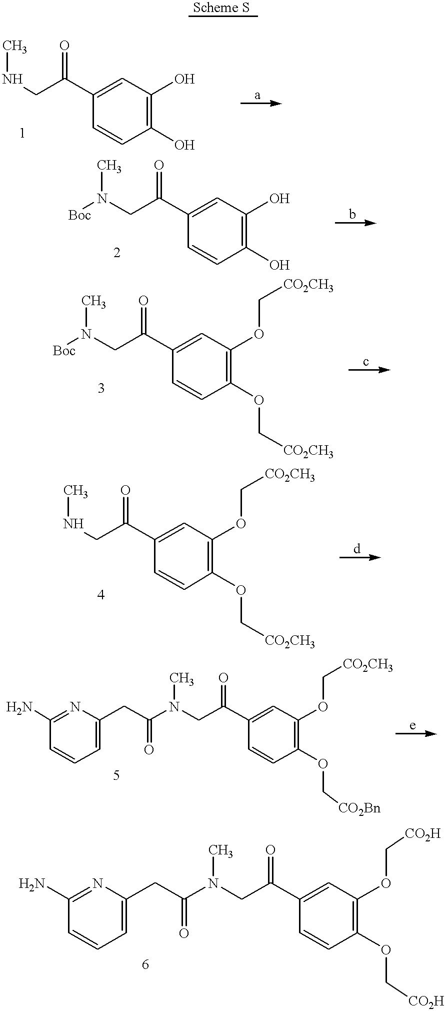

a) (Boc)₂O, NaOH, dioxane, H₂O; b) BrCH₂CO₂CH₃, K₂CO₃, acetone; c) 4M HCl, dioxane; d) (6-amino-2-pyridinyl)acetic acid, EDC, DIEA, DMF; e) 1N NaOH, CH₃OH.

S-1 is treated with di-tert-butyl dicarbonate and sodium hydroxide in aqueous dioxane to afford S-2, which is alkylated on the phenolic oxygens with methyl bromoacetate and potassium carbonate in acetone to give S-3. The Boc group in S-3 is removed with hydrogen chloride in dioxane, and the resulting S-4 is acylated on nitrogen with (6-amino-2-pyridinyl)acetic acid, prepared by saponification of ethyl (6-amino-2-pyridinyl)acetate, Awaya, et al., Chem Pharm. Bull. 1974, 22, 1414, EDC and DIEA in DMF to give S-5. The methyl esters in R-5 are cleaved by treatment with 1M NaOH in CH₃OH to give S-6.

Scheme T describes a method of preparing exemplary fibrinogen receptor templates described in Himmelsbach, et al., EP 0587134.

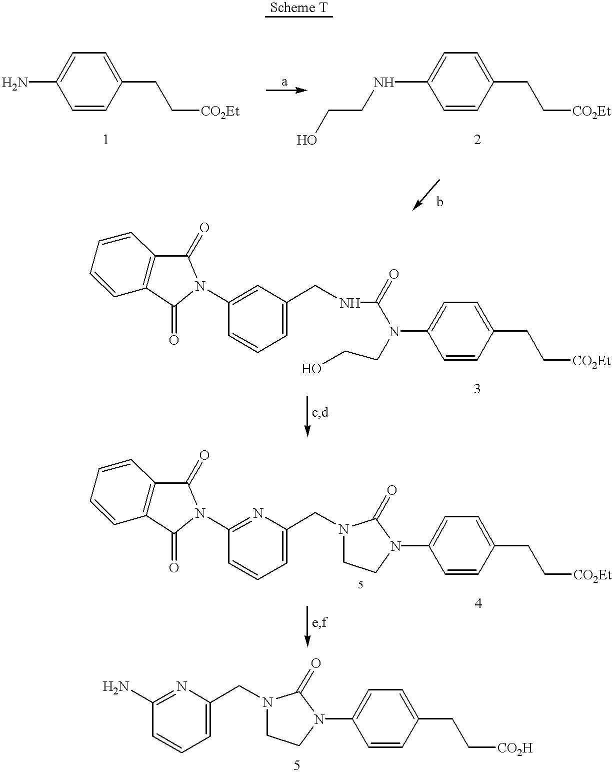

a) glycolaldehyde dimer, NaBH₃CN, H₂O, CH₃CN, pH 6-7; b) (6-phthalimido-2-pyridinyl)methanamine, COCl₂; c) CH₃SO₂Cl, Et₃N, CH₂Cl₂; d) NaI, KN(TMS)₂ THF, acetone, reflux; e) NH₂NH₂ H₂O; f) 1N NaOH, EtOH.

Scheme T provides a method for the preparation of 2-oxo-imidazolidine compounds, e.g., T-5, wherein reductive amination of an amine, for example T-1, with glycolaldehyde dimer and sodium cyanoborohydride, gives a secondary amine, such as T-2. A primary amine, as exemplified by (6-phthalimido-2-pyridinyl)methanamine, is treated with phosgene to give an isocyanate, which is allowed to react, without isolation, with the secondary hydroxyethylamine to give a hydoxyethylurea, as exemplified by compound T-3. The hydroxyl group is converted into a leaving group, such as a methanesulfonate or iodide, and is allowed to cyclize to a 2-oxo-imidazolidine, T-4, employing methods known in the art, Himmelsbach, et al., EP 0587134, such as treating the hydroxyethylurea 4 with trifluorosulfonyl chloride and Et₃N, followed by NaI and then potassium bis(trimethylsilyl)azide, as described in Himmelsbach, et al., EP 0587134, Example III. Treatment of T4 with hydrazine and saponification of the ester give T-5.

Scheme U provides a method for the preparation of 1,2,3,4-tetrahydroisoquinoline compounds as exemplary fibrinogen receptor antagonists, as described in M. J. Fisher et al., EP 0635492.

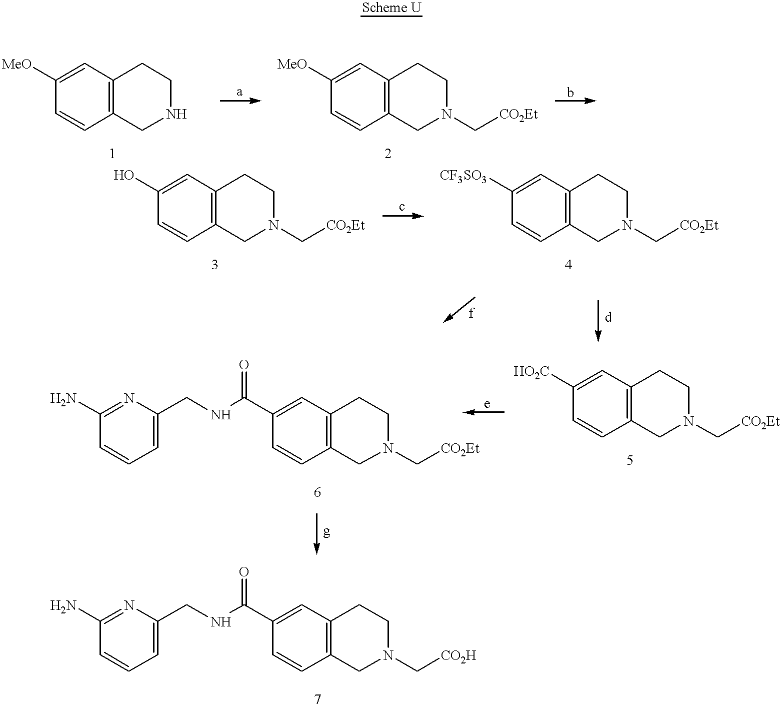

a) ClCH₂CO₂Et, Et₃N, DMF; b) BBr₃, CH₂Cl₂; c) (CF₃SO₂)₂O, pyridine; d) CO, Pd(OAc)₂, PPh₃, DIEA, NMP, NH₄HCO₃, H₂O; e) (6-amino2-pyridinyl)methanamine, EDC, HOBt, DIEA, DMF; f) (6-amino-2-pyridinyl)methanamine, CO, Pd(OAc)₂, PPh₃, DIEA, NMP, NH₄HCO₃, H₂O; g) 1N NaOH, EtOH.

Accordingly, a 6-methoxy-3,4-dihydroisoquinoline, such as compound U-1 is prepared by the method described by D. J. Sall and G. L. Grunewald, J. Med. Chem. 1987, 30, 2208-2216. The isoquinoline is treated with a haloacetic acid ester in the presence of a tertiary amine to afford the 2-acetic acid ester, as exemplified by compound U-2. The 6-methoxy compound is converted into the corresponding 6-hydroxy compound by methods known in the art, for example with BBr3, which is converted into the triflate with trifluorosulfonic acid anhydride. Palladium-catalyzed carbonylation affords the 6-carboxy compound, such as compound U-5, which is then condensed with an amine, as exemplified by (6-amino-2-pyridinyl)methanamine, employing a standard amide bond forming reagent to give the desired amide, such as compound U-6. Saponification affords the title compound of Example W, U-7. Alternatively, the palladium-catalyzed carbonylation reaction with the triflate, exemplified by compound U4, may be trapped with (6-amino-2-pyridinyl)methanamine to provide, after saponification, the compound of Example W, U-7.

Scheme V provides a method for the preparation of 3,4-dihydroisoquinolin-1-one compounds as exemplary fibrinogen receptor antagonists, as described M. J. Fisher et al., EP 0635492.

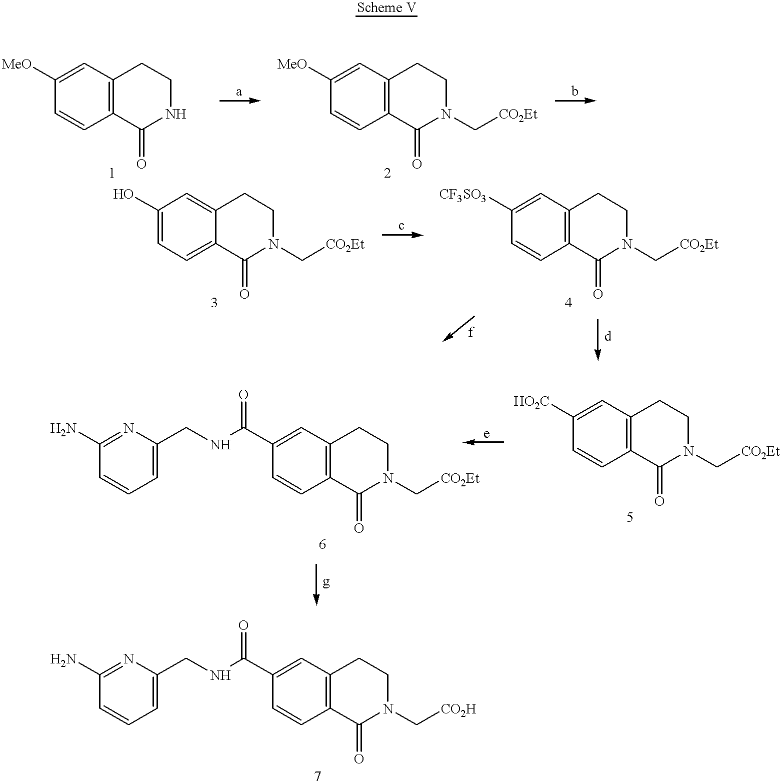

a) 1. LiN(TMS)₂, 2. ClCH₂CO₂Et, DMF; b) BBr₃, CH₂Cl₂; c) (CF₃SO₂)₂O, pyridine; d) CO, Pd(OAc)₂, PPh₃, DIEA, NMP, NH₄HCO₃, H₂O; e) N-(2-pyridinyl)ethylenediamine, EDC, HOBt, DIEA, DMF; f) (6-amino-2-pyridinyl)methanamine, CO, Pd(OAc)₂, PPh₃, DIEA, NMP, NH₄HCO₃, H₂O; g) 1N NaOH, EtOH.

Accordingly, the 1-oxo compound V-1, prepared by the method described by D. J. Sall and G. L. Grunewald, J. Med. Chem. 1987, 30, 2208-2216, is treated with a base, such as LiN(TMS)₂, and a haloacetic acid ester to give a 2-acetic acid ester, as exemplified by compound V-2. The 1-oxo compound is then employed in the analogous series of reactions deployed in Scheme U, substituting the corresponding 1-oxo analog, as shown in Scheme U, to provide the title compound of Example X, V-7. As in Scheme U, alternatively, the palladium-catalyzed carbonylation reaction with the triflate, exemplified by compound V-4, may be trapped with an amine, such as (6-amino-2-pyridinyl)methanamine, provides, after saponification, the amide exemplified by the title compound of Example X, V-7.

Scheme W provides a method for the preparation of 6-acylaminotetralin compounds as exemplary fibrinogen receptor antagonists, as described M. J. Fisher et al., EP 0635492.

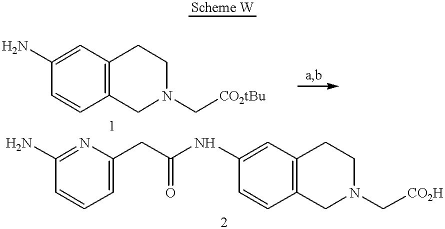

a) (6-amino-2-pyridinyl)acetic acid, EDC, HOBt, DIEA, DMF; b) TFA, CH₂Cl₂.

Accordingly, a 6-amino-2-tert-butyloxycarbonyl-tetral-1-one, exemplified by compound W-1, which is prepared according to the methods described in M. J. Fisher et al., EP 0635492, is condensed with an activated derivative of a carboxylic acid obtained (6-amino-2-pyridinyl)acetic acid to provide, after deesterification, the amide exemplified by the title compound of Example Y, W-2.

Scheme X provides a method for the preparation of 6-aminoacyltetralin compounds as exemplary fibrinogen receptor antagonists, as described M. J. Fisher et a., EP 0635492.

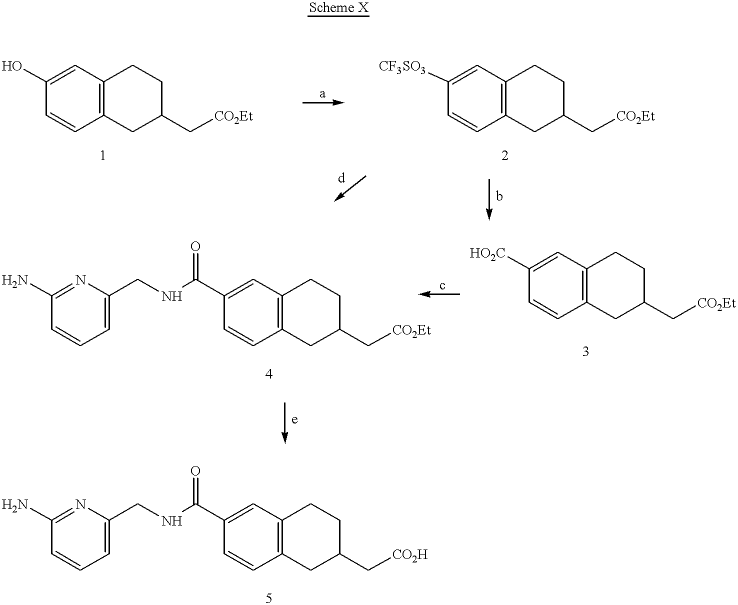

a) (CF₃SO₂)O, pyridine; b) CO, Pd(OAc)₂, PPh₃, DIEA, NMP, NH₄HCO₃, H₂O; c(6-amino-2-pyridinyl)methanamine, EDC, HOBt, DIEA, DMF; d) (6amino-2-pyridinyl)methanamine, CO, Pd(OAc)₂, PPh₃, DIEA, NMP, NH₄HCO₃, H₂O; e) 1N NaOH, EtOH.

Accordingly, an ethoxycarbonylmethyl-6-hydroxy-tetral-1-one, exemplified by compound X-1, which is prepared according to the methods described in M. J. Fisher et al., EP 0635492, is treated with triflic anhydride to provide the triflate, as exemplified by compound X-2, which is employed in a palladium-catalyzed carbonylation reaction to afford a carboxylic acid, such as compound X-3, which is then condensed with an amine such as (6-amino-2-pyridinyl)methanamine to provide, after deesterification, the 6aminoacyl compound exemplified by Example Z, X-5. Alternatively, the palladium-catalyzed carbonylation reaction with the triflate exemplified by compound X-2, may be trapped with (6-amino-2-pyridinyl)methanamine to provide, after saponification, the corresponding 6-aminoacyl compound X-5.

Scheme Y provides a method for the preparation of 5-acylaminobenzofuran and 5-acylaminodihydrobenzofuran compounds as exemplary fibrinogen receptor antagonists, as described in M. L. Denney, et al., EP 0655439.

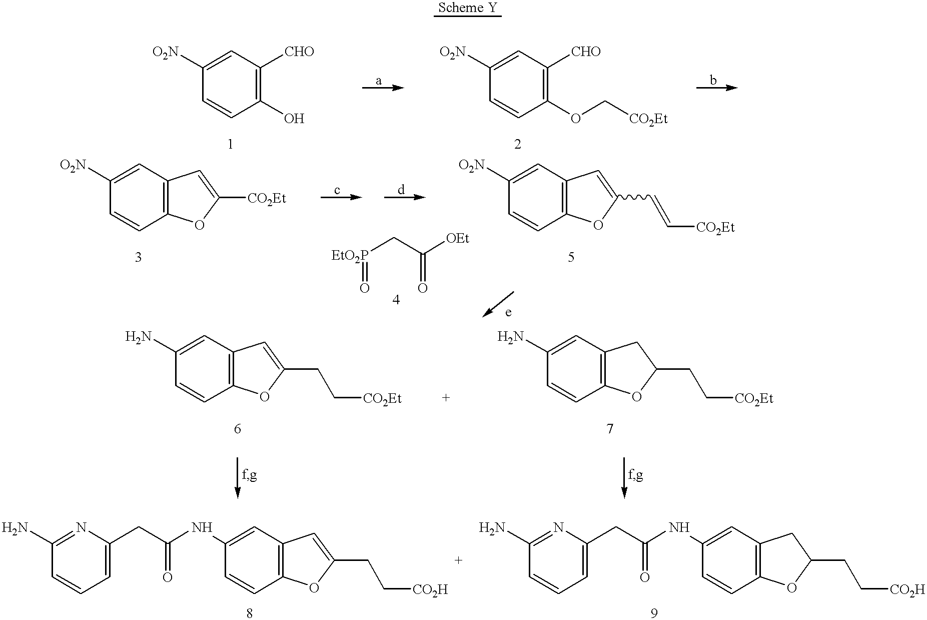

a) BrCH₂CO₂Et, K₂CO₃, NaI, THF; b) 1. DBU, EtOH, 2. HCl, EtOH; c) DiBAL, −78° C., THF; d) NaH, THF; e) H₂, 10% Pd/C, EtOH; f) (6-amino-2-pyridinyl)acetic acid, EDC, HOBT, Et₃N, DMF; g) 1N NaOH, CH₃OH.

Accordingly, a 5-nitrosalicylaldehyde, exemplified by compound Y-1, is treated with a haloacetic acid ester to give the phenoxyacetic acid ester, exemplified by compound Y-2. A 2-alkoxycarbonylfuran, exemplified by compound Y-3, is obtained by treating the aldehyde with base, for example with DBU. The 2-alkoxycarbonyl group is reduced to the aldehyde, for example with DiBAL. Wittig reaction affords the 2-acrylate ester, exemplified by compound Y-5, which is reduced to the benzofuran-2-propionic acid ester, exemplified by compound Y-6 and the dihydrobenzofuran-2-propionic acid ester, exemplified by compound Y-7. The amine Y-6 is then condensed with an activated derivative of a carboxylic acid, such as (6-amino-2-pyridinyl)acetic acid, to provide, after deesterification, the amide exemplified by the title compound of Example AA, Y-8. Alternatively, the amine Y-7 is condensed with an activated derivative of a carboxylic acid, such as (6-amino-2-pyridinyl)acetic acid, to provide, after deesterification, the amide Y-9.

Schemes Z-1, Z-2 and Z-3 provide a method for the preparation of 5-aminoacylbenzofuran and 5-aminoacyldihydrobenzofuran compounds as exemplary fibrinogen receptor antagonists, as described in M. L. Denney, et al., EP 0655439.

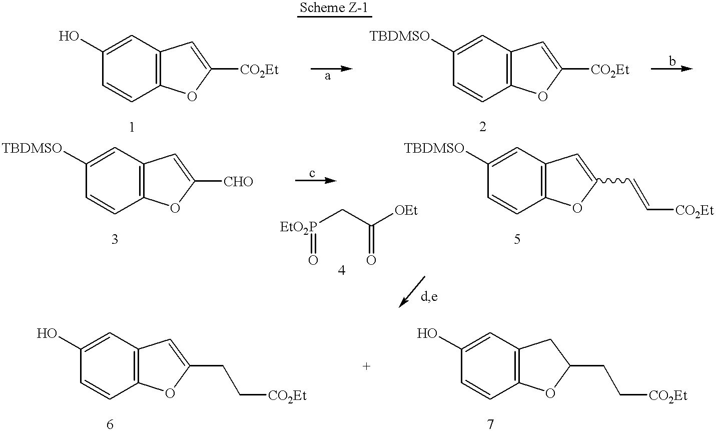

a) TBDMS-Cl, imidazole, THF; b) DiBAL, −78° C., THF; c) NaH, THF; d) H₂, 5% Pd/C, EtOH; e) Et4NF, THF.

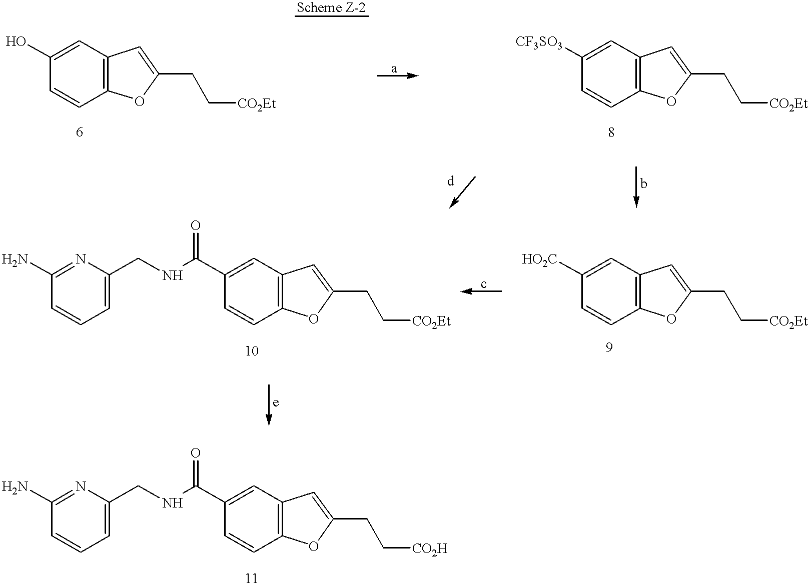

a) (CF₃SO₂)₂O, pyridine; b) CO, Pd(OAc)₂, PPh₃, DIEA, NMP, NH₄HCO₃, H₂O; c) (6-amino-2-pyridinyl)methanamine, EDC, HOBt, DIEA, DMF; d) (6-amino-2-pyridinyl)methanamine, CO, Pd(OAc)₂, PPh₃, DIEA, NMP, NH₄CO₃, H₂O e) 1N

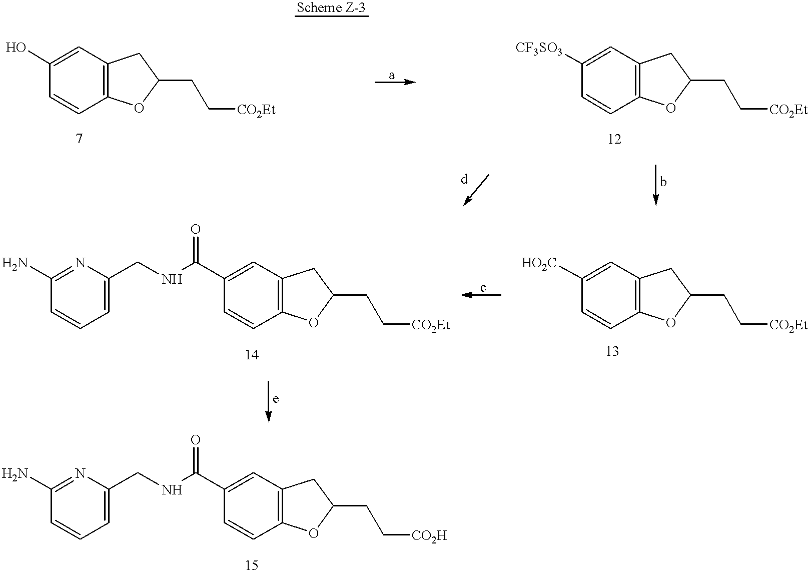

a) (CF₃SO₂)₂O, pyridine; b) CO, Pd(OAc)₂, PPh₃, DIEA, NMP, N₄CO₃, H₂O; c) (6-amino-2-pyridinyl)methanamine, EDC, HOBt, DIEA, DMF; d) (6-amino2-pyridinyl)methanamine, CO, Pd(OAc)₂, PPh₃, DIEA, NMP, NH₄HCO₃, H₂O; e) 1N NaOH, EtOH.

Accordingly, a 5-hydroxybenzofuran-2-carboxylic acid ester, such as compound Z-1-1, prepared in the manner of M. L. Denney, et al., EP 0655439, is treated with TBDMS-Cl to provide the TB1DMS derivative of the ester, Z-1-2. The ester is reduced to an aldehyde, such as compound Z-1-3. Wittig reaction affords an acrylic acid ester, as exemplified by compound Z-1-5. Catalytic reduction affords a benzofuran-2-acetic acid ester and a dihydrobenzofuran-2-acetic acid ester. Cleavage of the silyl ether group of each ester, by methods known to the art, affords a benzofuran-2-acetic acid ester, as exemplified by compound Z-1-6 and a dihydrobenzofuran-2-acetic acid ester as exemplified by compound Z-1-7.

As shown in Schemes Z-2 and Z-3, each phenol may be converted to a carboxylic acid via palladium-catalyzed carbonylation, such as compound Z-2-9 or Z-3-13, which are then condensed with an amine, such as (6-amino-2-pyridinyl)methanamine, to provide, after deesterification, the amide of the title compound of Example CC (Z-2-11) or DD (Z-3-15). Alternatively, the palladium-catalyzed carbonylation reaction with the triflates exemplified by compounds Z-2-8, or Z-3-12, may be trapped with (6-amino-2-pyridinyl)methanamine to provide, after deesterification, the corresponding 6-aminoacyl compounds, Example CC (Z-2-11) or DD (Z-3-15).

Scheme AA describes a method of preparing a further exemplary fibrinogen receptor template.

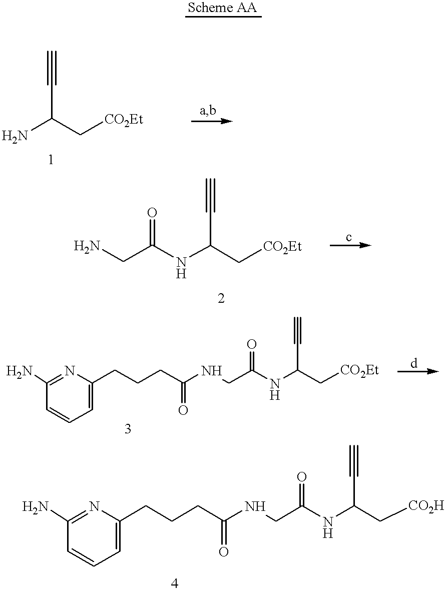

a) Boc-Gly, EDC, HOBT, DIEA, CH₃CN; b) TFA, CH₂Cl₂; c) 4-(6-amino-2-pyridinyl)butanoic acid, EDC, HOBT, DIEA, DMF; d) 1N LiOH, TBF, CH₃CN.

The preparation of the intermediate AA-2 begins with the coupling of the known ethyl 3-amino-4-pentynoate (WO 93/07867) with commercially available tert-butoxycarbonylglycine (Boc-Gly) under standard peptide bond forming conditions. The product of this reaction is deprotected to AA-2 under acidic conditions which are known to effect removal of a Boc protecting group. The two intermediates AA-2 and 4-(6-amino-2-pyridinyl)butanoic acid are coupled under standard peptide coupling conditions to give AA-3, which is hydrolyzed to AA⁴ with lithium hydroxide in aqueous THF and CH₃CN.

Acid addition salts of the compounds are prepared in a standard manner in a suitable solvent from the parent compound-and an excess of an acid, such as hydrochloric, hydrobromic, hydrofluoric, sulfuric, phosphoric, acetic, trifluoroacetic, maleic, succinic or methanesulfonic. Certain of the compounds form inner salts or zwitterions which may be acceptable. Cationic salts are prepared by treating the parent compound with an excess of an alkaline reagent, such as a hydroxide, carbonate or alkoxide, containing the appropriate cation; or with an appropriate organic amine. Cations such as Li⁺, Na⁺, K⁺, Ca⁺⁺, Mg⁺⁺ and NH₄⁺ are specific examples of cations present in pharmaceutically acceptable salts.

This invention also provides a pharmaceutical composition which comprises a compound according to formula (I) and a pharmaceutically acceptable carrier. Accordingly, the compounds of formula (I) may be used in the manufacture of a medicament. Pharmaceutical compositions of the compounds of formula (I) prepared as hereinbefore described may be formulated as solutions or lyophilized powders for parenteral administration. Powders may be reconstituted by addition of a suitable diluent or other pharmaceutically acceptable carrier prior to use. The liquid formulation may be a buffered, isotonic, aqueous solution. Examples of suitable diluents are normal isotonic saline solution, standard 5% dextrose in water or buffered sodium or ammonium acetate solution. Such formulation is especially suitable for parenteral administration, but may also be used for oral administration or contained in a metered dose inhaler or nebulizer for insufflation. It may be desirable to add excipients such as polyvinylpyrrolidone, gelatin, hydroxy cellulose, acacia, polyethylene glycol, mannitol, sodium chloride or sodium citrate.

Alternately, these compounds may be encapsulated, tableted or prepared in a emulsion or syrup for oral administration. Pharmaceutically acceptable solid or liquid carriers may be added to enhance or stabilize the composition, or to facilitate preparation of the composition. Solid carriers include starch, lactose, calcium sulfate dihydrate, terra alba, magnesium stearate or stearic acid, talc, pectin, acacia, agar or gelatin. Liquid carriers include syrup, peanut oil, olive oil, saline and water. The carrier may also include a sustained release material such as glyceryl monostearate or glyceryl distearate, alone or with a wax. The amount of solid carrier varies but, preferably, will be between about 20 mg to about 1 g per dosage unit. The pharmaceutical preparations are made following the conventional techniques of pharmacy involving milling, mixing, granulating, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

For rectal administration, the compounds of this invention may also be combined with excipients such as cocoa butter, glycerin, gelatin or polyethylene glycols and molded into a suppository.

The compounds described herein are antagonists of the vitronectin receptor, and are useful for treating diseases wherein the underlying pathology is attributable to ligand or cell which interacts with the vitronectin receptor. For instance, these compounds are useful for the treatment of diseases wherein loss of the bone matrix creates pathology. Thus, the instant compounds are useful for the treatment of ostoeporosis, hyperparathyroidism, Paget's disease, hypercalcemia of malignancy, osteolytic lesions produced by bone metastasis, bone loss due to immobilization or sex hormone deficiency. The compounds of this invention are also believed to have utility as antitumor, antiinflammatory, anti-angiogenic and anti-metastatic agents, and be useful in the treatment of cancer, atherosclerosis and restenosis.

The peptide is administered either orally or parenterally to the patient, in a manner such that the concentration of drug is sufficient to inhibit bone resorption, or other such indication. The pharmaceutical composition containing the peptide is administered at an oral dose of between about 0.1 to about 50 mg/kg in a manner consistent with the condition of the patient. Preferably the oral dose would be about 0.5 to about 20 mg/kg. For acute therapy, parenteral administration is preferred. An intravenous infusion of the peptide in 5% dextrose in water or normal saline, or a similar formulation with suitable excipients, is most effective, although an intramuscular bolus injection is also useful. Typically, the parenteral dose will be about 0.01 to about 100 mg/kg; preferably between 0.1 and 20 mg/kg. The compounds are administered one to four times daily at a level to achieve a total daily dose of about 0.4 to about 400 mg/kg/day. The precise level and method by which the compounds are administered is readily determined by one routinely skilled in the art by comparing the blood level of the agent to the concentration required to have a therapeutic effect.

The compounds may be tested in one of several biological assays to determine the concentration of compound which is required to have a given pharmacological effect.

### INHIBITION OF VITRONECTIN BINDING

Solid-Phase [³H]-SK&F-107260 Binding to αᵥβ₃: Human placenta or human platelet αᵥβ₃ (0.1-0.3 mg/mL) in buffer T (containing 2 mM CaCl₂ and 1% octylglucoside) was diluted with buffer T containing 1 mM CaCl₂, 1 mM MnCl₂, 1 mM MgCl₂ (buffer A) and 0.05% NaN₃, and then immediately added to 96-well ELISA plates (Corning, New York, N.Y.) at 0.1 mL per well. 0.1-0.2 μg of αᵥβ₃ was added per well. The plates were incubated overnight at 4° C. At the time of the experiment, the wells were washed once with buffer A and were incubated with 0.1 mL of 3.5% bovine serum albumin in the same buffer for 1 hr at room temperature. Following incubation the wells were aspirated completely and washed twice with 0.2 mL buffer A.

Compounds were dissolved in 100% DMSO to give a 2 mM stock solution, which was diluted with binding buffer (15 mM Tris-HCl (pH 7.4), 100 mM NaCl, 1 mM CaCl₂, 1 mM MnCl₂, 1 mM MgCl₂) to a final compound concentration of 100 μM. This solution is then diluted to the required final compound concentration. Various concentrations of unlabeled antagonists (0.001-100 μM were added to the wells in triplicates, followed by the addition of 5.0 nM of [³H]-SK&F-107260 (65-86 Ci/mmol).

The plates were incubated for 1 hr at room temperature. Following incubation the wells were aspirated completely and washed once with 0.2 ml of ice cold buffer A in a well-to-well fashion. The receptors were solubilized with 0.1 mL of 1% SDS and the bound [³H]-SK&F-107260 was determined by liquid scintillation counting with the addition of 3 mL Ready Safe in a Beckman LS Liquid Scintillation Counter, with 40% efficiency. Nonspecific binding of [³H]-SK&F-107260 was determined in the presence of 2 μM SK&F-107260 and was consistently less than 1% of total radioligand input. The IC₅₀ (concentration of the antagonist to inhibit 50% binding of [³H]-SK&F-107260) was determined by a nonlinear, least squares curve-fitting routine, which was modified from the LUNDON-2 program. The Kᵢ (dissociation constant of the antagonist) was calculated according to the equation: Kᵢ=IC₅₀/((1+L/K_{d}), where L and K_{d} were the concentration and the dissociation constant of [³H]-SK&F-107260, respectively.

Compounds of the present invention inhibit vitronectin binding to SK&F 107260 in the concentration range of 0.01 to 25 micromolar. Preferred compounds inhibit vitronectin binding at a concentration of less than 1 micromolar.

Compounds of this invention are also tested for in vitro and in vivo bone resorption in assays standard in the art for evaluating inhibition of bone formation, such as the pit formation assay disclosed in EP 528 587, which may also be performed using human osteoclasts in place of rat osteoclasts, and the ovarectomized rat model, described by Wronski et al., Cells and Materials 1991, Sup. 1, 69-74.

### PARATHYROIDECTOMIZED RAT MODEL

Each experimental group consists of 5-6 male Sprague-Dawley rats. The rats are parathyroidectomized (by the vendor, Taconic Farms) 7 days prior to use. Twenty four hours prior to use, circulating ionized calcium levels are measured in whole blood immediately after it has been withdrawn by tail venipuncture into heparinized tubes. Rats are included if ionized Ca level (measured with a Ciba-Corning model 634 calcium pH analyzer) is ²1.2 mM/L. The rats are then put on a diet of calcium-free chow and deionized water. At the start of the experiment the rats weigh approximately 100 g. Baseline Ca levels are measured and the rats are administered control vehicle (saline) or compound (dissolved in saline) as a single intravenous (tail vein) bolus injection followed immediately by a single subcutaneous injection of either human parathyroid hormone 1-34 peptide (hPTH1-34, dose 0.2 mg/kg in saline/0.1% bovine serum albumen, Bachem, Ca) or the PTH vehicle. The calcemic response to PITH (and any effect of compound on this response) is measured 2 h after compound/PH administration.

### RAT ULNA DRIFT MODEL

Each experimental group consists of 8-10 male Sprague-Dawley or Wistar rats of approximately 30-40 g body weight at the start of the experiment. The agent being tested is administered by an appropriate route as single or multiple daily doses for a period of seven days. Prior to administration of the first dose, the rats are given a single dose of a fluorescent marker (tetracycline 25 mg/kg, or calcein 10 mg/kg) that labels the position of bone forming surfaces at that point in time. After dosing of compound has been completed, the rats are killed and both forelimbs are removed at the elbow, the foot is removed at the ankle and the skin removed. The sample is frozen and mounted vertically on a microtome chuck. Cross sections of the midshaft region of the ulna are cut in the cryostat. The rate of bone resorption is measured morphometrically in the medial-dorsal portion of the cortical bone. The measurement is done as follows: the amount of bone resorbed at the periosteal surface is equal to the distance by which the periosteal surface has advanced towards the fluorescent label which had been incorporated at the endosteal bone formation surface on day zero; this distance is calculated by subtracting the width of bone between the label and the periosteal surface on day 7 from the width on day zero; the resorption rate in microns per day is calculated by dividing the result by 7.

### HUMAN OSTEOCLAST RESORPTION ASSAY (“PIT ASSAY”)

Aliquots of osteoclastoma-derived cell suspensions are removed from liquid nitrogen strorage, warmed rapidly at 37° C. and washed ×1 in RPMI-1640 medium by centrifugation (1000 rpm, 5 mins at 4° C).

Aspirate the medium and replace it with murine anti-HLA-DR antibody, diluted 1:3 in RPMI-1640 medium. Incubate for 30 mins on ice and mix the cell suspension frequently.

The cells are washed ×2 with cold RPMI-1640 by centrifugation (1000 rpm, 5 mins at 4° C.) and the cells are transferred to a sterile 15 ml centrifuge tube. The number of mononuclear cells are enumerated in an improved Neubauer counting chamber.

Sufficient magnetic beads (5/mononuclear cell), coated with goat anti-mouse IgG, are removed from their stock bottle and placed into 5 ml of fresh medium (this washes away the toxic azide preservative). The medium is removed by immobilizing the beads on a magnet and is replaced with fresh medium.

The beads are mixed with the cells and the suspension is incubated for 30 mins on ice. The suspension is mixed frequently.

The bead-coated cells are immobilized on a magnet and the remaining cells (osteoclast-rich fraction) are decanted into a sterile 50 ml centrifuge tube. Fresh medium is added to the bead-coated cells to dislodge any trapped osteoclasts. This wash process is repeated ×10. The bead-coated cells are discarded.

The osteoclasts are enumerated in a counting chamber, using a large-bore disposable plastic pasteur to charge the chamber with the sample.

The cells are pelleted by centrifugation and the density of osteoclasts adjusted to 1.5×10⁴/ml in EMEM medium, supplemented with 10% fetal calf serum and 1.7 g/liter of sodium bicarbonate.

3 ml aliquots of the cell suspension (per treatment) are decanted into 15 ml centrifuge tubes. The cells are pelleted by centrifugation.

To each tube 3 ml of the appropriate treatment are added (diluted to 50 uM in the EMEM medium). Also included are appropriate vehicle controls, a positive control (87MEM1 diluted to 100 ug/ml) and an isotype control (IgG2a diluted to 100 ug/ml). Incubate at 37° C. for 30 mins.

0.5 ml aliquots of the cells are seeded onto sterile dentine slices in a 48-well plate and incubated at 370° C. for 2 hours. Each treatment is screened in quadruplicate.

The slices are washed in six changes of warm PBS (10 ml/well in a 6-well plate) and then placed into fresh treatment or control. Incubate at 370° C. for 48 hours. tartrate resistant acid phosphatase (trap) procedure (selective stain for cells of the osteoclast lineage).

The slices are washed in phosphate buffered saline and fixed in 2% gluteraldehyde (in 0.2M sodium cacodylate) for 5 mins.

They are washed in water and incubated in TRAP buffer for 5 mins at 370° C.

Following a wash in cold water they are incubated in cold acetate buffer I fast red garnet for 5 mins at 4° C.

Excess buffer is aspirated, and the slices are air dried following a wash in water.

The TRAP positive osteoclasts are enumerated by bright-field microscopy and are then removed from the surface of the dentine by sonication.

Pit volumes are determined using the Nikon/Lasertec ILM21W confocal microscope.

### INHIBITION OF RGD-MEDIATED GPIIB-IIIA BINDING

### Purification of GPIIb-IIIa

Ten units of outdated, washed human platelets (obtained from Red Cross) were lyzed by gentle stirring in 3% octylglucoside, 20 mM Tris-HCl, pH 7.4, 140 mM NaCl, 2 mM CaCl₂ at 4° C. for 2 h. The lysate was centrifuged at 100,000 g for 1 h. The supernatant obtained was applied to a 5 mL lentil lectin sepharose 4B column (E.Y. Labs) preequilibrated with 20 mM Tris-HCl, pH 7.4, 100 mM NaCl, 2 mM CaCl₂, 1% octylglucoside (buffer A). After 2 h incubation, the column was washed with 50 m3L cold buffer A. The lectin-retained GPIIb-IIIa was eluted with buffer A containing 10% dextrose. All procedures were performed at 4° C. The GPIIb-IIIa obtained was >95% pure as shown by SDS polyacrylamide gel electrophoresis.

### Incorporation of GPIIb-IIIa in Liposomes

A mixture of phosphatidylserine (70%) and phosphatidylcholine (30%) (Avanti Polar Lipids) were dried to the walls of a glass tube under a stream of nitrogen. Purified GPIIb-IIIa was diluted to a final concentration of 0.5 mg/mL and mixed with the phospholipids in a protein:phospholipid ratio of 1:3 (w:w). The mixture was resuspended and sonicated in a bath sonicator for 5 min. The mixture was then dialyzed overnight using 12,000-14,000 molecular weight cutoff dialysis tubing against a 1000-fold excess of 50 mM Tris-HCl, pH 7.4, 100 mM NaCl, 2 mM CaCl₂ (with 2 changes). The GPIIb-IIIa-containing liposomes were centrifuged at 12,000 g for 15 min and resuspended in the dialysis buffer at a final protein concentration of approximately 1 mg/mL. The liposomes were stored at −70° C. until needed.

### Competitive Binding to GPIIb-IIIa

The binding to the fibrinogen receptor (GPIIb-IIIa) was assayed by an indirect competitive binding method using [³H]-SK&F-107260 as an RGD-type ligand. The binding assay was performed in a 96-well filtration plate assembly (Millipore Corporation, Bedford, Mass.) using 0.22 um hydrophilic durapore membranes. The wells were precoated with 0.2 mL of 10 μg/mL polylysine (Sigma Chemical Co., St. Louis, Mo.) at room temperature for 1 h to block nonspecific binding. Various concentrations of unlabeled benzadiazapines were added to the wells in quadruplicate. [³H]-SK&F-107260 was applied to each well at a final concentration of 4.5 nM, followed by the addition of 1 μg of the purified platelet GPIIb-IIIa-containing liposomes. The mixtures were incubated for 1 h at room temperature. The GPIIb-IIIa-bound [3H]-SK&F-107260 was seperated from the unbound by filtration using a Millipore filtration manifold, followed by washing with ice-cold buffer (2 times, each 0.2 mL). Bound radioactivity remaining on the filters was counted in 1.5 mL Ready Solve (Beckman Instruments, Fullerton, Calif.) in a Beckman Liquid Scintillation Counter (Model LS6800), with 40% efficiency. Nonspecific binding was determined in the presence of 2 μM unlabeled SK&F-107260 and was consistently less than 0.14% of the total radioactivity added to the samples. All data points are the mean of quadruplicate determinations.

Competition binding data were analyzed by a nonlinear least-squares curve fitting procedure. This method provides the IC50 of the antagonists (concentration of the antagonist which inhibits specific binding of [³H]-SK&F-107260 by 50% at equilibrium). The IC50 is related to the equilibrium dissociation constant (Ki) of the antagonist based on the Cheng and Prusoff equation: Ki=IC50/(1+L/Kd), where L is the concentration of [3H]-SK&F-107260 used in the competitive binding assay (4.5 nM), and Kd is the dissociation constant of [3H]-SK&F-107260 which is 4.5 nM as determined by Scatchard analysis.

Preferred compounds of this invention have an affinity for the vitronectin receptor relative to the fibrinogen receptor of greater than 4:1. More preferred compounds have a ratio of activity of greater than 10:1.

Vascular smooth muscle cell migration assay

The compounds of the instant invention were tested for their ability to inhibit the migration and proliferation of smooth muscle tissue in an artery or vein in order to assess their ability to prevent restenosis of an artery, such as that which typically occurs following angioplasty.

Rat or human aortic smooth muscle cells were used. The cell migration was monitored in a Transwell cell culture chamber by using a polycarbonate membrane with pores of 8 um (Costar). The lower surface of the filter was coated with vitronectin. Cells were suspended in DMEM supplemented with 0.2% bovine serum albumin at a concentration of 2.5-5.0×10⁶ cells/mL, and were pretreated with test compound at various concentrations for 20 min at 20° C. The solvent alone was used as control. 0.2 mL of the cell suspension was placed in the upper compartment of the chamber. The lower compartment contained 0.6 mL of DMEM supplemented with 0.2% bovine serum albumin. Incubation was carried out at 37° C. in an atmosphere of 95% air/5% CO₂ for 24 hr. After incubation, the non-migrated cells on the upper surface of the filter were removed by gentle scraping. The filter was then fixed in methanol and stained with 10% Giemsa stain. Migration was measured either by a) counting the number of cells that had migrated to the lower surface of the filter or by b) extracting the stained cells with 10% acetic acid followed by determining the absorbance at 600 nM.

### EXAMPLES

Nuclear magnetic resonance spectra were recorded at either 250 or 400 MHz using, respectively, a Bruker AM 250 or Bruker AC 400 spectrometer. CDCl₃ is deuteriochloroform, DMSO-d₆ is hexadeuteriodimethylsulfoxide, and CD₃OD is tetradeuteriomethanol. Chemical shifts are reported in parts per million (δ) downfield from the internal standard tetramethylsilane. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz. Infrared (IR) spectra were recorded on a Perkin-Elmer 683 infrared spectrometer in transmission mode. IR band positions are reported in inverse wavenumbers (cm⁻¹). Mass spectra were taken on either VG 70 FE, PE Syx API III, or VG ZAB BF instruments, using fast atom bombardment (FAB) or electrospray (S) ionization techniques. Elemental analyses were obtained using a Perkin-Elmer 240C elemental analyzer. Melting points were taken on a Thomas-Hoover melting point apparatus and are uncorrected. All temperatures are reported in degrees Celsius.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. Analytical and preparative HPLC were carried out on Rainin or Beckman chromatographs. ODS refers to an octadecylsilyl derivatized silica gel chromatographic support. 5μ Apex-ODS indicates an octadecylsilyl derivatized silica gel chromatographic support having a nominal particle size of 5μ, made by Jones Chromatography, Littleton, Colorado. YMC ODS-AQ® is an ODS chromatographic support and is a registered trademark of YMC Co. Ltd., Kyoto, Japan. PRP-1® is a polymeric (styrene-divinylbenzene) chromatographic support, and is a registered trademark of Hamilton Co., Reno, Nev. Celite® is a filter aid composed of acid-washed diatomaceous silica, and is a registered trademark of Manville Corp., Denver, Colo.

Methyl (±)-7-carboxy-2,3,4,5-tetrahydro-4-methyl-3-oxo-1H-1,4-benzodiazepine-2-acetate and methyl (±)-7-carboxy-2,3,4,5-tetrahydro-3-oxo-4-phenylethyl-1H-1,4-benzodiazepine-2-acetate was prepared by the method of Bondinell et al. WO 93/00095. Tert-butyl 3-(bromomethyl)-4-fluorobenzoate and methyl (S)-7-carboxy-2,3,4,5-tetrahydro-4-methyl-3-oxo-1H-1,4-benzodiazepine-2-acetate was prepared by the method of Bondinell et al. WO 95/18619.

### PREPARATION OF INTERMEDIATE COMPOUNDS

### Preparation A

### Preparation of Benzyl 3-[3,4-dihyro-8-carboxy-1-methyl-2,5-dioxo-1H-1,4-benzodiazepine]-4-propanoate

a) 4-Iodo-2-amino benzoic acid

Oxidation of 4-iodo-2-nitrotoluene according to Sasson, et. al., J. Org. Chem. 1986, 51, 2880-83, to give 4-iodo-2-nitro benzoic acid followed by reduction of the nitro group using iron and acetic acid gives the title compound.

b) 7-Iodoisatoic anhydride

To a mechanically stirred ice cold solution of the compound of Preparation A(a) (26.3 g, 0.1 mol), Na₂CO₃ (10.6 g, 0.1 mol) and H₂O (250 mL), is slowly added, via an addition funnel, a solution of 1.93M COCl₂ in toluene (80 mL). After 2 h, the precipitated product is isolated by filtration, and the solid is washed successively with H₂O (200 mL), a 1:1 mixture of EtOH:Et₂O (300 mL), and Et₂O (200 mL), and dried under vacuum to yield the title compound.

c) Benzyl N-(2-amino-4-iodobenzoyl)-β-alanine

A magnetically stirred solution of the compound of Preparation A(b) (5.0 g, 0.0173 mol), β-alanine benzyl ester tosylate (5.85 g, 0.0173 mol), and DMAP (0.5 g, 0.0041 mol) in pyridine (35 mL) is heated for 2 h at 80° C. The reaction mixture is allowed to cool to RT and concentrated. The resulting residue is dissolved in EtOAc (100 mL), and washed successively with 10% cupric sulfate (2×50 mL), saturated NaHCO₃ (1×50 mL) and brine (1×50 mL), dried (Na₂SO₄), filtered, and concentrated to afford the title compound after chromatography (silica gel, 1:1 EtOAc/hexanes).

d) Benzyl N-(2-methylamino-4-iodobenzoyl)-β-alanine

A magnetically stirred solution of the compound of Preparation A(c) (2.0 mmol), 2,6-lutidine (0.35 mL, 3.0 mmol) and CH₃I (0.19 mL, 3.0 mmol) in DMF (15 mL) is heated at 50° C. for 15 h. The reaction mixture is allowed to cool to RT and concentrated. The resulting residue is dissolved in EtOAc (75 mL), and washed successively with 10% citric acid (1×50 mL), saturated NaHCO₃ (1×50 i) and brine (1×50 mL), dried (Na₂SO₄), filtered, and concentrated to afford the title compound after chromatography (silica gel, gradient, 35-65% EtOAc/hexanes).

e) Benzyl 3-[3,4-dihyro-8-iodo-1-methyl-2,5-dioxo-1H-1,4-benzodiazepine]-4-propanoate

To a cold (−30° C.) magnetically stirred solution of the compound of Preparation A(d) (0.305 g, 0.69 mmol), Et₃N (0.144 g, 1.04 mmol) in CH₂CO₂ (3 mL) is added slowly a solution of α-bromoacetyl bromide (0.09 mL, 1.04 mmol) in CH₂Cl₂ (2 mL) under argon atmosphere. The reaction mixture is allowed to warm to RT and stir for 2 h. The mixture is diluted with CH₂Cl₂ (40 mL) and washed successively with 10% citric acid (1×50 mL), saturated NaHCO₃ (1×50 mL), dried (Na₂SO₄), filtered, and concentrated. The resulting residue is dissolved in DMF (3 mL) and added via an addition funnel to a slurry of NaH (25 mg, 1.04 mmol) in DMF (2 mL) which is cooled to 0° C. After 2 h of stirring, the mixture is poured into an ice cold solution of 10% citric acid (50 mL) and extracted with EtOAc (3×40 mL). The combined extracts are washed with saturated NaHCO₃ (1×50 mL), dried (Na₂SO₄), filtered, and concentrated to afford the title compound after chromatography (silica gel, gradient, 40-70% EtOAc/hexanes).

f) Benzyl 3-[3,4-dihyro-8-carboxy-1-methyl-2,5-dioxo-1H-1,4-benzodiazepine]-4-propanoate

A solution of the compound of Preparation A(e) (3.2 mmol), Pd(OAc)₂ (0.16 mmol), and 1,1′-bis(diphenylphosphine)ferrocene (0.64 mmol, ) in DMSO (20 mL) is heated to 65° C. under a carbon monoxide balloon for 18 h. The reaction mixture is diluted with water, acidified with 1N HCl and extracted with CH₂Cl₂. The combined organic extracts are washed with water, dried (Na₂SO₄), filtered, and concentrated to afford the title compound after chromatography (silica gel).

### Preparation B

### Ethyl 3-[4H-imidazo[1,2-a][1,4]benzodiazepine-5(6H)-1-methyl-1-6oxo-9-carboxy]-5-propanoic acid

a) Ethyl N-(2-amino-4-iodobenzoyl)-β-alanine

A magnetically stirred solution of the compound of Preparation A(b) (0.0173 mol), β-alanine ethyl ester hydrochloride (0.0173 mol), and DMAP (0.5 g, 0.0041 mol) in pyridine (35 mL) is heated for 2 h at 80° C. The reaction mixture is allowed to cool to RT and concentrated. The resulting residue is dissolved in EtOAc (100 mL), and washed successively with 10% cupric sulfate (2×50 mL), saturated NaHCO₃ (1×50 mL) and brine (1×50 mL), dried (Na₂SO₄), filtered, and concentrated to afford the title compound after chromatography (silica gel, 1:1 EtOAc/hexanes).

b) Ethyl 3-[3,4-dihyro-8-iodo-2,5-dioxo-1H-1,4-benzodiazepine]-4-propanoate

To a cold (−30° C.) magnetically stirred solution of the compound of Preparation B(a) (0.69 mmol), and Et₃N (0.144 g, 1.04 mmol) in CH₂Cl₂ (3 mL) is added slowly a solution of α-bromoacetyl bromide (0.09 mL, 1.04 mmol) in CH₂Cl₂ (2 mL) under argon atmosphere. The reaction mixture is allowed to warm to RT and stir for 2 h. The mixture is diluted with CH₂Cl₂ (40 mL) and wash successively with 10% citric acid (1×50 mL), saturated NaHCO₃ (1×50 mL), dried (Na₂SO₄), filtered, and concentrated. The resulting residue is dissolved in DMF (3 mL) and added via an addition funnel to a slurry of NaH (25 mg, 1.04 mmol) in DMF (2 mL) which is cooled to 0° C. After 2 h of stirring, the mixture is poured into an ice cold solution of 10% citric acid (50 mL) and extracted with EtOAc (3×40 mL). The combined extracts are washed with saturated NaHCO₃ (1 ×50 mL), dried (Na₂SO₄), filtered, and concentrated to afford the title compound after chromatography (silica gel).

c) Ethyl-3-[3,4dihyro-8-iodo-2-thioxo-5-oxo-1H-1,4-benzodiazepine]-4-propanoate

To a solution of the compound of Preparation B(b) (1.0 g, 2.49 mmol) in TBF (10 mL) at RT and under an atmosphere of nitrogen is added Lawesson's reagent (1.0 g) and the reaction is heated at 50° C. for 2 h. The reaction mixture is allowed to cool to RT and is concentrated. Purifying the resulting residue by chromatography (silica gel, gradient, 40-60% EtOAc/hexane) gives the title compound.

d) Ethyl 3-[4H-imidazo[1,2-a][1,4]benzodiazepine-5(6H)-1-methyl-6-oxo-9-iodo]-5-propanoate

To a vigorously stirred biphasic solution of the compound of Preparation B(c) (0.95 g, 2.27 mmol), CH₃I (0.2 g) and a catalytic amount of tetrabutylammonium hydrogen sulfate in CH₂Cl₂ (10 mL) and H₂O (10 mL), is added 2 N NaOH (1.2 mL) at RT. After 2 h, the layers are separated and the aqueous layer is washed with CH₂Cl₂ (2×25 mL). The combined organic extracts are dried (Na₂SO₄), filtered, and concentrated. The resulting residue is dissolved in toluene (10 mL) and allowed e react with propargyl amine (0.64 mL) and pyridine hydrochloride (0.23 g). The reaction is heated to reflux for 6 h, allowed to cool to RT, and concentrated to give the title compound after chromatography (silica gel, EtOAc).

e) Ethyl 3-[4H-imidazo[1,2-a][1,4]benzodiazepine-5(6H)-1-methyl-6oxo-9-carboxy]-5-propanoic acid

A solution of the compound of Preparation B(d) (3.2 mmol), Pd(OAc)₂ (0.16 mmol), and 1,1′-bis(diphenylphosphine)ferrocene (0.64 mmol) in DMSO (20 mL) is heated at 65° C. under a carbon monoxide balloon for 18 h. The reaction mixture is diluted with H₂O, acidified with 1N HCl and extracted with CH₂Cl₂ (3×). The combined organic extracts are washed with H₂O, dried (Na₂SO₄), filtered, and concentrated to afford the title compound after chromatography (silica gel).

### Preparation C

### Preparation of Ethyl 4-(1-piperazinyl)-1-piperidineacetate

a) Ethyl 4-[4-(tert-butoxycarbonyl)-1-piperazinyl]-1-piperidineacetate

The titled compound is prepared from tert-butyl 1-piperazinecarboxylate (Aldrich) and ethyl 4-oxo-1-piperidineacetate (Porter, et. al., EP 0 542 363 A2) by reductive amination with NaBH₃CN according to the method of Porter, et. al., EP 0 542 363 A2.

b) Ethyl 4-(1-piperazinyl)-1-piperidineacetate

A solution of Preparation C(a) and 4M HCl in dioxane/CH₂Cl₂ is stirred at RT for 18 h. The reaction mixture is concentrated to give the title compound as the hydrochloride salt.

### Preparation D

### Preparation of 6-Methyl-2-(phthalimido)pyridine

A mixture of 6-methyl-2-aminopyridine and neat phthalic anhydride is heated for 5 h, diluted with aqueous NaHCO₃, and extracted with EtOAc. The combined extracts are dried and evaporated to give the title compound.

### Preparation E

### Preparation of 3-(6-Amino-2-pyridinyl)propanol

a) 1-[6-(3-Hydroxypropyl)-2-pyridinyl]-2,5-dimethylpyrrole

Following the procedure of Warter, et. al., Org. Synth. 1943, 23, 83, for the preparation of 2-(3-hydroxypropyl)pyridine, except substituting 1-(6-methylpyridin-2-yl)-2,5-dimethylpyrrole for the 2-methylpyridine, J. Chem. Soc., Perkin Trans. 1, 1984, 2801-2807, gives the title compound.

b) 3-(6-Amino-2-pyridinyl)propanol

Following the procedure of Bruekelman, et. al., J. Chem. Soc. Perkin Trans. 1, 1984, 2801-2807, for the preparation of 2-amino-6-ethylpyridine, except substituting the compound of Preparation E(a) for 1-(6-ethylpyridin-2-yl)-2,5-dimethylpyrrole, gives the title compound.

### Preparation F

### Preparation of 4-[6-(Toluenesulfonylamino)-2-pyridinyl]-1-propanol oxime

a) 4-[6-(2,5-Dimethylpyrrol-1-yl)-2-pyridinyl]-1-butene

Following the procedure of Meakins, J. Chem. Soc. Perkin Trans. 1, 1984, 2801, for the alkylation of 6-(2,5-diethylpyrrol-1-yl)-2-picoline, except using alkyl bromide as the alkylating agent, the title compound is prepared.

b) 4-(6-Amino-2-pyridinyl)-1-butene

Following Procedure B, described Meakins, J. Chem. Soc. Perkin Trans. I, 1984, 2801, the compound of Preparation F(a) is deprotected to give the title compound.

c) 4-[6-(Toluenesulfonylamino)-2-pyridinyl]-1-butene

Sodium hydride (55 mmol) is added carefully to a solution of the compound of Preparation F(b) (50 mmol) and 4toluenesulfonyl chloride (55 mmol) in dry THF (200 mL). The reaction is stirred at RT until complete, then is quenched with saturated NH₄Cl (200 mL), and the mixture is extracted with EtOAc. The combined organic extracts are dried (MgSO₄) and concentrated, and the residue is purified by chromatography (silica gel) to give the title compound.

d) 4-[6-(Toluenesulfonylamino)-2-pyridinyl]-1-propanal

Ozone is bubbled into a solution the compound of Preparation F(c) (40 mmol) in CH₂Cl₂ (160 mL) and CH₃OH (40 ml) at −78° C. until the blue color persists, then the excess ozone is removed by bubbling argon through the solution. Dry dimethylsulfide (excess) is added, and the reaction is warmed to RT. The reaction is stirred at RT until complete, then is concentrated, and the residue is chromatographed (silica gel) to afford the title compound.

e) 4-[6-(Toluenesulfonylamino)-2-pyridinyl]-1-propanal oxime

Hydroxylamine hydrochloride (33 mmol) is added to a solution of the compound of Preparation F(d) (30 mmol) and anhydrous NaOAc (66 mmol) in CH₃OH (150 mL) at 0° C. The reaction is stirred at 0° C. until complete, then is concentrated, and the residue is partitioned between H₂O and EtOAc. The layers are separated, and the aqueous layer is extracted with EtOAc. The combined organic layers are washed sequentially with 5% NaHCO₃ and saturated brine, dried (MgSO₄), and concentrated to afford the title compound.

### Preparation G

### Preparation of (6-Amino-2-pyridinyl)acetic acid

Ethyl (6-amino-2-pyridinyl)acetate, Awaya, et. al., Chem. Pharm. Bull., 1974, 22, 1414, (1 mmol) is treated with 1N NaOH (1.5 mmol) in CH₃OH (20 mL). The mixture is concentrated, extracted with CH₂Cl₂, and the aqueous phase is adjusted to pH 5 to give the title compound.

### Preparation H

Preparation of 6-(2-Aminoethyl)-2-pyridinamine dihydrochloride Following the procedures of Preparation 13 in Bondinell, et al., WO 94/14476, for the preparation of 2-aminopyridine-4-ethanamine dihydrochloride, except substituting 2-(acetylamino)pyridine-6-carboxylic acid for the 2-(acetylamino)pyridine-4-carboxylic acid, the title compound is prepared.

### Preparation I

### Preparation of 4-[(6-Amino-2-pyridinyl)methyl]phenol

4-[(6-Amino-2-pyridinyl)methyl]anisole, Ife et. al., WO 9426715, is heated with concentrated hydrobromic acid to afford the title compound.

### Preparation J

### Preparation of Benzyl 4-[2-(methylamino)acetyl]phenoxyacetate hydrochloride

a) 4-[N-Boc-2-(methylamino)acetyl]phenol

A solution of di-tert-butyl dicarbonate (5.96 g, 27.3 mmol) in 1,4-dioxane (25 mL) was added dropwise at 0° C. to a mixture of 4-[2-(methylamino)acetyl]phenol hydrochloride (5.0 g, 24.8 mmol), 1,4-dioxane (30 mL), H₂O (25 mL), and 1.0N NaOH (25 mL, 25 mmol). After 24 h, the reaction was warmed to RT and stirred for 1.5 h. More 1.0N NaOH (25 mL, 25 mmol) was added, and the reaction was stirred for an additional 0.5 h at RT, and concentrated. The residue was diluted with EtOAc (80 mL), and the mixture was acidified to pH 2 using 1.0M NaHSO₄. The resulting mixture was extracted with EtOAc, and the combined organic layers were washed with H₂O and dried (Na₂SO₄). Filtration and concentration gave the title compound (6.49 g, 99%): ¹H NMR (250 MHz, CDCl₃) δ6.70-8.05 (m, 4H), 4.53 (s, 2H), 2.98 (s, 3H), 1.50 (s, 9H).

b) Benzyl 4-[N-Boc-2-(methylamino)acetyl]phenoxyacetate

A mixture of the compound of Preparation J(a) (5.04 g, 19.0 mmol) and K₂CO₃ (2.63 g, 19.0 mmol) in acetone (100 mL) was stirred at reflux under argon for 1 h. The mixture was cooled to RT and benzyl bromoacetate (5.23 g, 22.8 mmol) was added. The reaction was heated at reflux for 18 h, then was cooled and filtered. The filter cake was washed with acetone, and the filtrate was concentrated. The residue was dissolved in CH₂Cl₂ (300 mL) and washed sequentially with H₂O (50 mL) and brine (50 mL). Drying (Na₂SO₄), concentration, and flash chromatography (silica gel, 1:3 EtOAc/hexanes) yielded the title compound (7.28 g, 93%): ¹H NMR (250 MHz, CDCl₃) δ6.85-7.95 (m, 9 H), 5.23 (s, 2H), 4.71 (s, 2H), 4.55 (d, 2H), 2.95 (d, 3H), 1.45 (d, 9H).

c) Benzyl 4-[2-(methylamino)acetyl]phenoxyacetate hydrochloride

A mixture of the compound of Preparation J(b) (7.26 g, 17.57 mmol) and 4M HCl in 1,4-dioxane (150 mL) was stirred for 1 h at RT. Concentration and trituration with Et₂O afforded the title compound as a white powder (5.93 g, 97%): ¹H NMR (250 MHz, CD₃OD) δ7.05-8.00 (m, 9 H), 5.23 (s, 2H), 4.88 (s, 2H), 4.65 (s, 2H), 2.80 (s, 3H).

### Preparation K

### Preparation of Dimethyl 4-[2-(methylamino)acetyl]-1,2-phenylenedioxydiacetate hydrochloride

a) 4-[N-Boc-2-(methylamino)acetyl]-1,2-dihydroxybenzene

Following the procedure of Preparation J(a), except substituting adrenalone hydrochloride (5.0 g, 23.0 mmol) for 4-[2-(methylamino)acetyl]phenol hydrochloride, the title compound (1.2 g, 19%) was prepared following flash chromatography (silica gel, 1:1 EtOAc/hexanes): MS (ES) m/e 282.2 [M+H]⁺.

b) Dimethyl 4-[N-Boc-2-(methylamino)acetyl]-1,2-phenylenedioxydiacetate

Following the procedure of Preparation J(b), except substituting the compound of Preparation K(a) (0.9 g, 3.2 mmol) for the compound of Preparation J(a) and methyl bromoacetate (1.23 g, 8.0 mmol) for benzyl bromoacetate, the title compound (1.11 g, 81%) was prepared: MS (ES) m/e 426.2 [M+H]⁺.

c) Dimethyl 4-[N-Boc-2-(methylamino)acetyl]-1,2-phenylenedioxydiacetate hydrochloride

Following the procedure of Preparation J(c), except substituting the compound of Preparation K(b) (1.11 g, 2.6 mmol) for the compound of Preparation J(b), the title compound was prepared (1.1 g, quantitative): MS (ES) m/e 326.0 [M+H]⁺.

### Preparation L

### Preparation of (6-Phthaloyl-2-pyridinyl~)methanamine

Following the procedure of Preparation T, except substituting ammonia for methylamine, gives the title compound.

### Preparation M

### Preparation of Ethyl (6-carboxy-1,2,3,4-tetrahydroisoquinolin-2-yl)acetate

a) Ethyl (6-Methoxy-1,2,3,4-tetrahydroisoquinolin-2-yl)acetate

A solution of 6methoxy-1,2,3,4-tetrahydroisoquinoline, Sall and Grunewald, J. Med. Chem. 1987, 30, 2208-2216, (1.1 mmol), ethyl chloroacetate (1.17 mmol), and K₂CO₃ (1.17 mmol) in CH₃CN (10 mL) is stirred for 18 h. The mixture is partitioned between EtOAc and H₂0. The organic phase is concentrated to an oil, which is purified by chromatography (silica gel, gradient, 20-80% EtOAc/hexane) to afford the title compound.

b) Ethyl (6-Hydroxy-1,2,3,4-tetrahydroisoquinolin-2-yl)acetate

A solution of the compound of Preparation M(a)(0.249 g, 1.0 mmol), 1M BBr₃ in CH₂Cl₂ (1.0 mL, 1.0 mmol) is stirred at −70° C. for 2 h and then stirred at RT for 12 hr. The solution is concentrated, and the solution of the resulting oil in EtOAc is washed with H₂O, 5% NaHCO₃, and H₂O, dried (Mg₂SO₄), filtered, and concentrated to an oil to afford the title compound (0.223 g, 95%)

c) Ethyl [6-(trifluoromethylsulfonyloxy)-1,2,3,4-tetrahydroisoquinolin-2-yl]acetate

A solution of the compound of Preparation M(b)(0.235 g, 1.0 mmol), trifluorosulfonic acid anhydride (0.23 mL, 1.1 mmol,) and Et₃N (0.32 mL, 1.5 mmol) in CH₂Cl₂ (5 mL) is stirred for 8 h. The solution is concentrated to an oil which is taken up in EtOAc. The organic phase is washed with 5% NaHCO₃ and H₂O. The organic phase is dried (Na₂SO₄), filtered, concentrated to afford the title compound (0.300 g, 82%)

d) Ethyl (6-carboxy-1,2,3,4-tetrahydroisoquinolin-2-yl)acetate

A solution of the compound of Preparation M(c)(0.367 g, 1.0 mmol), Pd(OAc)₂ (0.022 g, 0.1 mmol,), Ph₃P (0.262 g, 1.0 mmol), diisopropylamine (0.34 mL, 2.5 mmol), and NMP (5 mL) in 10% NH₄CO₃ is stirred for 8 h under an atmosphere of CO. The solution is concentrated to an oil which is purified by chromatography (silica gel, gradient, 10-33% CH₃OH/CH₂Cl₂) to afford the title compound (0.19 g, 72%).

### Preparation N

### Preparation of Ethyl (6-carboxy-1,2,3,4-tetrahydro-1-oxo-isoquinoline-2-yl)acetate

a) Ethyl (6-Methoxy-1-oxo-1,2,3,4-tetrahydroisoquinolin-2-yl)acetate

A mixture of 6-methoxy-1,2,3,4-tetrahydro-1-oxo-isoquinoline, Sall and Grunewald, J. Med. Chem., 1987, 30, 2208-2216, (0.39 mmol) and NaH (0.17 g, 0.43 mmol, 60% oil dispersion) in THF (5 mL) is heated to reflux for 1 h and then allowed to cool to RT. Ethyl chloroacetate (0.43 mmol) is added and the mixture is allowed to stir for 1 h. The mixture is quenched with H₂O (10 mL) and washed with EtOAc. The organic layers are combined, washed with H₂O (10 mL) and concentrated to an oil which is purified by (silica gel, gradient, 10-33% CH₃OH/CH₂Cl₂) to afford the title compound.

b) Ethyl (6-Hydroxy-1-oxo-1,2,3,4-tetrahydroisoquinolin-2-yl)acetate

A solution of the compound of Preparation N(a) (0.263 g, 1.0 mmol) and 1M BBr₃ in CH₂Cl₂ (1.1 mL) is stirred at −70° C. for 2 h and then at RT for 4 h. The solution is concentrated to an oil which is taken up in EtOAc. The organic phase is washed with H₂O, 5% NaHCO₃, H₂O, dried (MgSO₄), filtered, and concentrated to afford the title compound (0.20 g, 80%).

c) Ethyl (6-(trifluoromethylsulfonyloxy)-1,2,3,4-tetrahydro-1-oxo-isoquinolin-2-yl]acetate

A solution of the compound of Preparation N(b) (3.4 mmol) and trifluorosulfonic acid anhydride (3.4 mmol, mL) in pyridine (5 mL) is chilled at 0° C. and allowed to warm RT for 1 h. The mixture is quenched with H₂O (5 mL) and washed with EtOAc. The organic layers are combined, washed with H₂O (7 mL) and concentrated to an oil. The residue is purified chromatography (by silica gel, gradient, 14-75% EtOAc/hexane) to afford the title compound.

d) Ethyl (6-carboxy-1-oxo-1,2,3,4-tetrahydroisoquinolin-2-yl)acetate

A solution of the compound of Preparation N(c) (0.23 g, 1.0 mmol), Pd(OAc)₂ (0.026 g, 0.1 mmol), Ph₃P (0.262 g, 1.0 mmol), diisopropylamine (0.23 mL, 2.0 mmol) and NMP (7 mL) in 10% NH₄CO₃ is stirred for 8 h under an atmosphere of CO. The solution is concentrated to an oil which is purified by chromatography (silica gel, gradient, 25-75% CH₃OH/CH₂Cl₂) to afford the title compound (0.31 g, 70%).

### Preparation O

### Preparation of Ethyl (6-carboxy-tetralin-2-yl)acetate

a) Ethyl [6-(trifluoromethylsulfonyloxy)-tetralin-2-yl]acetate

Following the procedure of Preparation M(c), except substituting ethyl (6-hydroxy-tetralin-2-yl)acetate, Fisher, et. al., EP 0635492, Scheme 6 and Example 20, parts A-D for the compound of Preparation M(b), gives the title compound.

b) Ethyl (6-carboxy-tetralin-2-yl)acetate

Following the procedure of Preparation M(d), except substituting the compound of Preparation O(a) for the compound of Preparation M(c), gives the title compound.

### Preparation P

### Preparation of Ethyl (5-aminobenzofuran-2-yl)propionate and Ethyl (5-amino-2,3-dihydro-benzofuran-2-yl]propionate

a) 2-(Ethoxycarbonyl)methoxy-5-nitrobenzaldehyde

A solution of 5-nitrosalicylaldehyde (Aldrich) (0.167 g, 1.0 mmol), ethyl bromoacetate (0.166 g, 1.0 mmol), K₂CO₃ (0.276 g, 2.0 mmol) and NaI (0.015 g, 0.1 mmol) in THF (10 mL) is heated to 80° C. for 24 h. The solution is concentrated and the residue is purified by chromatography (silica gel, gradient, 5-20% CH₃OH in CH₂Cl₂) to afford the title compound (0.20 g, 87%)

b) Ethyl (5-nitrobenzofuran-2-yl)carboxylate

A solution of the compound of Preparation P(a) (0.229 g, 1.0 mmol) and DBU (0.152 g, 1.0 mmol) in EtOH (10 mL) is allowed to stir at RT for 18 h. The solution is concentrated and the residue is treated with EtOH (10 mL). The solution is bubbled with HCl gas for 2 min and refluxed for 5 h. The solution is concentrated and the residue is treated with EtOAc. The organic phase is washed with H₂O, 5% citric acid, H₂O, 5% NaHCO₃, and H₂O. The organic phase is concentrated to afford the title compound (0.19 g, 81%).

c) Ethyl (5-nitrobenzofuran-2-yl)carboxaldehyde

A cold solution (−78° C.) of the compound of Example 20(b) (0.235 g, 1.0 mmol) in THF (5 mL) is treated with 1M DiBAL in THF (1.0 mL, 1.0 mmol). The solution is stirred at −78° C. for 30 min and at RT for 3 h. The solution is treated with CH₃CO₂H (3 mL) followed by H₂O (2 mL). The solution is concentrated and the residue is treated with toluene to azeotrope off the acetic acid. Drying in vacuo afforded the title compound (0.100 g, 52%)

d) Ethyl (5-nitrobenzofuran-2-yl)propenoate

A solution of triethyl phosphonoacetate (0.224 g, 1.0 mmol) in THF (5 mL) is treated with NaH (60% suspension in mineral oil, 0.04 g, 1.0 mmol) at 0° C. for 1 h. To the solution is added the compound of Preparation P(c)(0.235 g, 1.0 mmol). The solution is stirred at RT for 18 h, concentrated, and the residue is purified by chromatography (silica gel, gradient, 5-20% EtOAc/hexane) (EtOAc/Hexane 0.5:9 to 4:1) to afford the title compound (0.2 g, 77%).

e) Ethyl (5-aminobenzofuran-2-yl)propionate and Ethyl (5-amino-2,3-dihydrobenzofuran-2-yl]propionate

A solution of the compound of Preparation P(d) (0.261 g, 1.0 mmol) in EtOH (5 mL) containing 10% Pd/C (0.026 g) is hydrogenated at 45 psi for 1 h. The solution is filtered through Celite and the filtrate is concentrated and chromatographed (silica gel, gradient, 25-75% EtOAc/hexane) affords the title compounds.

### Preparation Q

### Preparation of Ethyl (5-carboxy-benzofuran-2-yl)propionate

a) Ethyl [5-(tert-butyldimethylsilyloxy)benzofuran-2-yl]carboxylate

A solution of ethyl [5-(hydroxy)benzofuran-2-yl]carboxylate, Denny, et. al., EP 0655439, (0.206 g, 1.0 mmol), tert-(butyl)dimethylsilyl chloride (0.23 mL, 1.0 mmol) and imidazole (0.34 g, 1.0 mmol) in THF is allowed to stir for 4 h. The solution is concentrated and the residue is treated with EtOAc. The organic phase is washed with H₂O, dried (Na₂SO₄), and concentrated to afford the tide compound (0.35 g, 90%).

b) Ethyl [5-[tert-(butyl)dimethylsilyloxy]benzofuran-2-yl]propenoate

Following the procedure of Preparation P(c) and (d), except substituting the compound of Preparation Q(a) for the compound of Preparation P(b), gives the title compound.

c) Ethyl [5-(hydroxy)benzofuran-2-yl]propionate and Ethyl [5-hydroxy-2,3-dihydro-benzofuran-2-yl]propionate

A mixture of the compound of Preparation Q(b) (0.234 g, 1.2 mmol) and 10% Pd/C (0.023 g, 10% wt) in EtOH(5 mL) is hydrogenated at 50 psi for 1 h. The mixture is filtered through Celite and concentrated. A solution of the residue (0.34 g, 1.0 mmol) and Et₄NF (0.149 g, 1.0 mmol) in THF (10 mL) is allowed to stir at RT for 18 h. The solution is concentrated and purified by chromatography (silica gel) to give the title compounds (0.25 g, 57%).

d) Ethyl [5-(trifluoromethylsulfonyloxy)benzofuran-2-yl]propionate

Following the procedure of Preparation M(c), except substituting ethyl [5-(hydroxy)benzofuran-2-yl]propionate of Preparation Q(c) for the compound of Preparation M(b), gives the title compound.

e) Ethyl (5-carboxy-benzofuran-2-yl)propionate

Following the procedure of Preparation M(d), except substituting the compound of Preparation Q(d) for the compound of Preparation M(c), gives the title compound.

### Preparation R

### Preparation of Ethyl (5-carboxy-2,3-dihydrobenzofuran-2-yl)propionate

a) Ethyl [5-(trifluoromethylsulfonyloxy)-2,3-dihydro-benzofuran-2-yl]propionate

Following the procedure of Preparation Q(d), except substituting ethyl [5-hydroxy-2,3-dihydro-benzofuran-2-yl]propionate from Preparation Q(c) for ethyl [5-(hydroxy)benzofuran-2-yl]propionate from Preparation Q(c), gives the title compound.

b) Ethyl (5-carboxy-2,3-dihydro-benzofuran-2-yl)propionate

Following the procedure of Preparation Q(e), except substituting the compound of Preparation R(a) for the compound of Preparation Q(d), gives the title compound.

### Preparation S

### Preparation of Ethyl (±)-3-[(glycyl)amino]-4-pentynoate trifluoroacetate

a) Ethyl (±)-3-[[(N-tert-butoxycarbonyl)glycyl]amino]-4-pentanoate

DIEA (0.92 mL, 5.32 mmol) was added to a stirred solution of ethyl (±)-3-amino-4-pentynoate (0.3 g, 2.13 mmol), Boc-Gly (0.56 g, 3.19 mmol), HOBt H₂O (0.43 g, 3.19 mmol), and EDC (0.61 g, 3.19 mmol) in anhydrous CH₃CN (15 mL) at RT. After 34 h, the reaction mixture was concentrated, diluted with CH₂Cl₂ (70 mL), and washed sequentially with 5% NaHCO₃ (2×15 mL) and brine (15 mL). Drying (MgSO₄), concentration, and chromatography (silica gel, 1:1 EtOAc/hexane) gave the title compound (0.5 g, 79%) as a colorless oil: MS (ES) m/e 299.2 (M+H)⁺.

b) Ethyl (±)-3-[(glycyl)amino]pentynoate trifluoroacetate

A solution of TFA (5 mL) and CH₂Cl₂ (15 mL) at RT was added all at once to the compound of Preparation S(a) (0.5 g, 1.68 mmol). After 30 min, the solution was concentrated, and the residue was reconcentrated from toluene (to remove residual TFA) to afford the title compound (0.55 g, 106%) as a light yellow syrup: MS (ES) m/e 199.2 (M+H)⁺.

### Preparation T

### Preparation of 6-(Methylamino)methyl-2-pyridinamine

6-Bromomethyl-2-(phthalimido)pyridine (prepared according to the method of U.S. Pat. No. 4,490,533)(1.1 g, 3 mmol) was added to a solution of ethanol (100 mL) saturated with methylamine at 0° C. The resulting solution was stirred at 0° C. for 2 h, concentrated to a volume of 20 mL, and treated with hydrazine hydrate (1 mL, 20 mmol). The resulting solution was heated to reflux for 2 h, concentrated, and the residue was chromatographed (silica gel; step gradient, 5%-15% CH₃OH/CH₂Cl₂) to give the title compound as a yellow oil (0.15 g, 32%): ¹H NMR (400 MHz, DMSO-d₆) δ7.34 (t, J=7.8 Hz, 1H), 6.55 (d, J=7.1 Hz, 1H), 6.47 (d, J=8.3 Hz, 1H), 3.93 (s, 3H), 2.61 (s, 3H).

### Preparation U

### Preparation of 6-Aminomethyl-2-pyridinamine

a) 2-(Phthalimido)methyl-6-(phthalimido)pyridine

A mixture of -6bromomethyl-2-(phthalimido)pyridine (U.S. Pat. No. 4,490,533)(0.4 g, 1.2 mmol), potassium phthalimide (0.30 g, 1.6 mmol), and DMF (4 mL) was stirred at RT for 18 h, concentrated, and the residue was partitioned between EtOAc and H₂0. The organic phase was washed with brine, dried (MgSO₄), and concentrated. The residue was recrystallized (CHCl₃) to give the title compound as a white solid (0.4 g, 83%): MS (ES) m/e 383.9 [M+H]⁺.

b) 6-Aminomethyl-2-pyridinamine

A solution of the compound of Preparation U(a)(0.4 g) and hydrazine hydrate (2 mL) in ethanol (10 mL) was heated to reflux for 2 h, filtered, and the filtrate was concentrated. The residue was triturated with CHCl₃, and the organic extracts were combined and concentrated to give the title compound as an amber oil (0.09 g, 70%): MS (ES) m/e 123.7 [M+H]⁺.

### Preparation V

### Preparation of N-Ethyl-6-(aminomethyl)-2-pyridinamine

6-(Acetylamino)picolinamide (Farmaco Ed. Sci., 1959, 14, 594) (0.3 g, 1.67 mmol) suspended in dry THF (5 mL) was added dropwise to a solution of 1M LAH in THF (16.7 mL) cooled to 0° C. The suspension was allowed to warm to RT and was heated to reflux for 4 h. The mixture was cooled, carefully treated with H₂0 and 10% NaOH, and filtered. The filtrate was dried (MgSO₄), concentrated, and the residue was azeotroped several times with toluene. The resulting mixture was concentrated to give the title compound (0.25 g, 99%): MS (ES) m/e 152 [M+H]⁺.

### Preparation W

### Preparation of 4-(Methylaminomethyl)-2-pyrimidinamine

A suspension of NaOAc (0.39 g, 4.8 mmol) in a solution of methylamine hydrochloride (0.39 g, 5.8 mmol) in EtOH (30 mL) was stirred 10 min at RT and 4 formyl-2-pyrimidinamine (obtained by the method of WO 9502591) (0.30 g, 2.4 mmol) was added in one portion. The mixture was stirred 30 min and NaBH₃CN (0.09 g, 1.44 mmol) was added. The resulting suspension was stirred 16 h, filtered, and concentrated. The residue was partitioned between CH₂Cl₂ (150 mL) and 5% Na₂CO₃ (20 mL). The aqueous layer was extracted with CH₂Cl₂ (4×25 mL), and the combined organic layers were dried (K₂CO₃) and concentrated to give the crude title compound (0.40 g): MS (ES) m/e 139 [M+H]⁺.

### EXAMPLES

### Example 1

### Preparation of (S)-7-[[[(6-Amino-2-pyridinyl)methyl]methylamino]carbonyl]-2,3,4,5-tetrahydro-4-methyl-3-oxo-1H-1,4-benzodiazepine-2-acetic acid

a) Methyl (S)-7-[[[(6-amino-2-pyridinyl)methyl]methylamino]carbonyl]-2,3,4,5-tetrahydro-4-methyl-3-oxo-1H-1,4-benzodiazepine-2-acetate

EDC (0.25 g, 1.3 mmol) was added to a solution of methyl (S)-7-carboxy-2,3,4,5-tetrahydro-4-methyl-3-oxo1H-1,4-benzodiazepine-2-acetate (0.32 g, 1.1 mmol), the compound of Preparation T (0.15 g, 6.02 mmol), HOBT.H₂O (170 mg, 1.3 mmol), and DIEA (0.9 mL, 4.4 mmol) in anhydrous CH₃CN (5 mL) at RT. After 21 h, the reaction was concentrated and the residue was partitioned between EtOAc and H₂O. The organic layer was washed with brine, dried (MgSO₄), and concentrated. The residue was purified by chromatography (silica gel, step gradient, 2%-7% CH₃O/CH₂Cl₂) to give the title compound (0.22 g, 48%): MS (ES) m/e 412.4 [M+H]⁺.

b) (S)-7-[[[(6-Amino-2-pyridinyl)methyl]methylamino]carbonyl]-2,3,4,5-tetrahydro-4-methyl-3-oxo-1H-1,4-benzodiazepine-2-acetic acid

A solution of the compound of Example 1(a) (0.22 g, 54 mmol), LiOH-H₂O (0.033 g, 0.79 mmol), TBF (5 mL), and water (2 mL) was stirred at RT overnight. The mixture was concentrated and the residue was dissolved in water. The resulting solution was brought to pH 5 with 3N HCl and allowed to stand. The crystals that formed were collected by filtration and dried to give the title compound as a pale yellow solid (0.125 g, 59%): MS (ES) m/e 398.4 [M+H]⁺. Anal. Calcd for C₂₀H₂₃N₅O₄.⅜ H₂O: C, 59.43; H, 5.92; N, 17.33. Found: C, 59.42; H, 5.73; N, 17.18.

### Example 2

### Preparation of (S)-7-[[[(6-Amino-2-pyridinyl)methyl]amino]carbonyl]-2,3,4,5-tetrahydro-4-methyl-3-oxo-1H-1,4-benzodiazepine-2-acetic acid

a) Methyl (S)-7-[[[(6-amino-2-pyridinyl)methyl]amino]carbonyl]-2,3,4,5-tetrahydro-4-methyl-3-oxo-1H-1,4-benzodiazepine-2-acetate

Following the procedure of Example 1(a), except substituting the compound of Preparation U(b) for the compound of Preparation T, gave the title compound as a white foam: MS (ES) m/e 398.0 (M+H]⁺.

b) (S)-7-[[[(6-Amino-2-pyridyl)methyl]amino]carbonyl]-2,3,4,5-tetrahydro-4-methyl-3-oxo-1H-1,4-benzodiazepine-2-acetic acid

Following the procedure of Example 1(b), except substituting the compound of Example 2(a) for the compound of Example 1(a), gave the title compound as a white solid: MS (ES) m/e 384.2 [M+H]⁺. Anal. Calcd. for C₁₉H₂₁N₅O₄.1.25 H₂O: C, 56.22; H, 5.83; N, 17.25. Found: C, 56.01; H, 5.99; N, 16.92.

### Example 3

### Preparation of (S)-7-[[[(6-Ethylamino-2-pyridinyl]methyl]amino]carbonyl]-2,3,4,5-tetrahydro-4-methyl-3-oxo-1H-1,4-benzodiazepine-2-acetic acid

a) Methyl (S)-7-[[[(6-ethylamino-2-pyridinyl)methyl]amino]carbonyl]-2,3,4,5-tetrahydro-4-methyl-3oxo-1H-1,4-benzodiazepine-2-acetate

A mixture of the compound of Preparation V (0.25 g, 1.65 mmol), methyl (S)-7-carboxy-2,3,4,5-tetrahydro-4-methyl-3-oxo-1H-1,4-benzodiazepine-2-acetate (0.58 g, 2 mmol), EDC (0.38 g, 2 mmol), and HOBT.H₂O (0.26 g, 2 mmol) in DMF (20 mL) was stirred at RT overnight. The mixture was concentrated, and the residue was treated with 5% Na₂CO₃ and extracted with CH₂Cl₂ (3×30 mL). The combined organic extracts were washed with H₂O, dried (MgSO₄), concentrated. The residue was chromatographed (silica gel, 5% CH₃OH/CH₂Cl₂) to give the title compound (0.16 g, 23%): MS (ES) m/e 426 [M+H]⁺.

b) (S)-7-[[[(6-Ethylamino-2-pyriinyl)methyl]amino]carbonyl]-2,3,4,5-tetrahydro-4-methyl-3-oxo-1H-1,4-benzodiazepine-2-acetic acid

The compound of Example 3(a) (0.16 g, 0.4 mmol) was dissolved in CH₃OH (10 mL) and THF (1 mL), and treated with 1N NaOH (0.5 mL). The mixture was stirred overnight, concentrated, and the residue was dissolved in H₂O and extracted with CH₂Cl₂. The pH of the aqueous phase was adjusted to 5.5-6 with dilute HCl, and the solid which formed was filtered, washed with H₂O and Et₂O, and dried to give the title compound (0.11 g, 73%): MS (ES) m/e 412 [M+H]⁺. Anal. Calcd for C₂₁H₂₅N₅O₄. 0.625 H₂O: C, 59.91; H, 6.08; N, 16.21. Found: C, 59.67; H, 6.26; N, 16.51.

### Example 4

### Preparation of (±)-7-[[[(2-Amino-4-pyrimidinyl)methyl]methylamino]carbonyl]-2,3,4,5-tetrahydro-4-methyl-3-oxo-1H-1,4-benzodiazepine-2-acetic acid

a) Methyl (±)-7-[[[(2-amino-4-pyrimidinyl)methyl]methylamino]carbonyl]-2,3,4,5-tetrahydro-4-methyl-3-oxo-1,4-benzodiazepine-2-acetate

A solution methyl (±)-7-(chlorocarbonyl)-2,3,4,5-tetrahydro-4-methyl-3-oxo-1H-1,4-benzodiazepine-2-acetate hydrochloride (0.43 g, 1.25 mmol) in CH₂Cl₂ (45 mL) was added dropwise to a solution of the compound of Preparation W (0.34 g, 2.5 mmol) and pyridine (0.60 g, 7.6 mmol) in CH₂Cl₂ (50 mL). The resulting suspension was stirred 20 h, filtered, and extracted with 5% Na₂CO₃ (30 mL). The organic layer was dried (Na₂SO₄) and concentrated to dryness to give a brown solid which was purified by preparative TLC R_{f} 0.58 (Whatman PLK5F, 10% CH₃OH/CH₂Cl₂) to give the title compound (0.38 g, 74%): MS (ES) m/e 413 [M+H]⁺.

b) (±)7-[[[(2-Amino-pyrimidinyl)methyl]methylamino]carbonyl]-2,3,4,5-tetrahydro-4-methyl-3-oxo-1H-1,4-benzodiazepine-2-acetic acid

A solution of the compound of Example 4(a) in a mixture of CH₃OH (30 mL) and 0.5N NaOH (6.0 mL) was heated at 50° C. for 2 h, cooled to RT, and treated with TFA (1.0 mL). The solution was evaporated to dryness and the residue was purified by HPLC t_{R} 21.0 min (ODS-AQ, 50×250 mm, 90 mL/min, 86:14 CH₃CN:H₂O-1% TFA, UV detection at 220 nm) to give the title compound (0.150 g): MS (ES) m/e 399[M+H]⁺.

### Example 5

### Preparation of 3-[3,4-Dihydro-8-[[[(6-amino-2-pyridinyl)methyl]methylamino]carbonyl]-1-methyl-2,5-dioxo-1H-1,4-benzodiazepine]-4-propanoic acid

a) Benzyl 3-(3,4-dihydro-8-[[[(6-amino-2-pyridinyl)methyl]methylamino]carbonyl]-1-methyl-2,5-dioxo-1H-1,4-benzodiazepine]-4-propanoate

EDC (0.25 g, 1.3 mmol) is added to a solution of the compound of Preparation A(f) (1.1 mmol), the compound of Preparation T (1.1 mmol), HOBT-H₂O (170 mg, 1.3 mmol), and DIEA (0.9 mL, 4.4 mmol) in anhydrous CH₃CN (5 mL) at RT. After 21 h, the reaction is concentrated and the residue is purified by chromatography (silica gel) to afford the title compound.

b) 3-[3,4-Dihydro-8-[[[(6-amino-2-pyridinyl)methyl]methylamino]carbonyl]-1-methyl-2,5-dioxo-1H-1,4benzodiazepine]-4-propanoic acid

A mixture of the compound of Example 5(a) (2 mmol) and 10% Pd/C (0.02 g) in EtOH (100 mL) is hydrogenated in an atmosphere of H₂ (50 psi) for 6 h. The catalyst is removed by filtration, and the filtrate is concentrated to afford the title compound.

### Example 6

### Preparation of 3-[4H-imidazo[1,2-a][1,4]benzodiazepine-5(6H)-1-methyl-6-oxo-9-[[[(6-amino-2-pyridinyl)methyl]methylamino]carbonyl]-5-propanoic acid

a) Ethyl 3-[4H-imidazo]1,2-a][1,4]benzodiazepine-5(6H)-1-methyl-6oxo-9-[[[(6-amino-2-pyridyl)methyl]-N-methylamino]carbonyl]-5-propanoic acid

EDC (0.25 g, 1.3 mmol) is added to a solution of the compound of Preparation B(e) (1.1 mmol), the compound of Preparation T (1.1 mmol), HOBT.H₂O (170 mg, 1.3 mmol), and DIEA (0.9 mL, 4.4 mmol) in anhydrous CH₃CN (5 mL) at RT. After 21 h, the reaction is concentrated and the residue is purified by chromatography (silica gel) to afford the title compound.

b) 3-[4H-imidazo(1,2-a][1,4]benzodiazepine-5(6H)-1-methyl-6oxo-9-[[[(6-amino-2-pyridyl)methyl]-N-methylamino]carbonyl]-5-propanoic acid

1M LiOH (3.8 mL, 3.8 mmol) is added dropwise to a solution of the compound of Example 6(a) (2.5 mmol) in 1:1 CH₃OH:THF (20 mL) at RT. The resulting mixture is stirred for 20 h and concentrated. The residue is dissolved in H₂O and acidified with TFA (20%) to afford the title compound after chromatography.

### Example 7

### Preparation of 4-[4-2-(6-Amino-2-pyridinyl)ethyl]-1-piperazinyl]-1-piperidineacetic acid

a) Ethyl 4-[4-[2-[6-(phthalimido)-2-pyridinyl]ethyl]-1-piperazinyl]-1-(piperidinyl)acetate

According to the methods of Shoeb, et. al., Pharmazie, 1978, 33, 581, and Finkelstein, et. al., J. Amer. Chem. Soc., 1951, 73, 302, a mixture compounds of Preparation C(b), Preparation D, and formaldehyde in EtOH is heated to reflux for 1 h, cooled, diluted with aqueous NaHCO₃, and extracted with EtOAc. The combined organic extracts are dried, evaporated, and flash chromatographed to give the title compound

b) 4- [4-2-[6-(Phthalimido)-2-pyridinyl]ethyl]-1-piperazinyl]-1-piperidineacetic acid

A solution of Example 7(a) in NaOH/CH₃OH is stirred at RT for 18 h. The mixture is neutralized with HOAc and filtered to give the title compound.

c) 4-[4-[2-(6-Amino-2-pyridinyl)ethyl]-1-piperazinyl]-1-piperidineacetic acid

A solution of Example 7(b) and hydrazine hydrate in EtOH is heated to reflux for 2 h.. The mixture is neutralised, desalted, and lyophilized to give the title compound.

### Example 8

### Preparation of 1-Hydroxyl-4-[4-[[6-amino-2-pyridinyl)methyl]-1-piperazinyl]-cyclohexaneacetic acid

a) 1,1-Dimethylethyl 1-hydroxyl-4-[4-[(6-phthalimido-2-pyridinyl)methyl]-1-piperazanyl]-cyclohexaneacetate

A mixture 1,1-dimethylethyl 1-hydroxyl-4-(1-piperazinyl)-cyclohexaneacetate, Porter, et. al., EP 0 537 980 A1, 6-bromomethyl-2-(phthalimido)pyridine, U.S. Pat. No. 4,490,533, and NaHCO₃ in CH₃CN is warmed. The mixture is concentrated and the residue is partitioned between H₂O and EtOAc. The organic phase is dried (Na₂SO₄), concentrated, and the residue is chromatographed (silica gel) to give the title compound.

b) 1-Hydroxyl-4-[4-[(6-phthalimido-2-pyridinyl)methyl]-1-piperazinyl]-cyclohexaneacetic acid

A solution of Example 8(b) in 4M HCl/dioxane/CH₂Cl₂ is stirred at RT for 18 h. The mixture is evaporated and filtered to give the title compound.

c) 1-Hydroxyl-4-[4-[(6-amino-2-pyridinyl)methyl]-1-piperazinyl]-cyclohexaneacetic acid

A solution of the compound of Example 8(b) in EtOH is treated with hydrazine hydrate and warmed. The mixture is concentrated and chromatographed (silica gel) to give the title compound.

### Example 9

### Preparation of 1-Hydroxyl-4-[4-[2-(6-amino-2-pyridinyl)ethyl]-1-piperazinyl]-cyclohexaneacetic acid

Following the general procedure of Example 7, except substituting 1,1-dimethylethyl 1-hydroxyl-4-(1-piperazinyl)-cyclohexaneacetate for the compound of Preparation C(b), gives the title compound.

### Example 10

### Preparation of 4-[4-[(6-Amino-2-pyridinyl)methyl]-1-piperazinyl]-1-piperidineacetic acid

Following the general procedure of Example 8, except substituting the compound of Preparation C(b) for 1,1-dimethylethyl 1-hydroxyl-4-(1-piperazinyl)-cyclohexaneacetate, gives the title compound.

### Example 11

### Preparation of N-[[1-[[2-(6-Amino-2-pyridinyl)ethyl]carbonyl]-3-piperidinyl]carbonyl]-β-alanine

Following the procedures of Beavers et. al., WO 95/25091, Example 1, except substituting (6-amino-2-pyridinyl)propionic acid, Bondinell, et. al., WO 94/14775, for N^{α}-Boc-D-lys(Cbz)—OH, gives the title compound.

### Example 12

### Preparation of 2-[(6-Amino-2-pyridinyl)methyl]-5-[2-(carboxy-ethyl)amino]carbonyl]-2,3-dihydro-3-oxo-1H-isoindole

Following the procedures of Preparation 1-12 in Hartman, et. al., EP 0 540 334 A1, for the preparation of 2,3-dihydro-N-(2-carboxy-ethyl)-2-[2-(piperidinyl)ethyl]-3-oxo-1H-isoindole-5-carboxamide, except substituting the compound of Preparation U for Boc-4-piperidine-2-ethylamine, gives the title compound.

### Example 13

### Preparation of (S)2-(Butylsulfonylamino)-3-[4-[[3-(6-amino-2-pridinyl) propyl]oxy]phenyl]propionic acid

Following the procedures of Egbertson, et al., EP 0478363 A2, for the preparation of 2-S-(butylsulfonylamino)-3-[4-(N-benzyloxycarbonylpiperidin-4-yl)-2,2-dimethyl]butyloxyphenylpropionic acid, except substituting the compound of Preparation E(b) for 4-[4-(N-benzyloxycarbonylpiperidin-4-yl)-2-methyl]pentan-2-ol in, gives the title compound.

### Example 14

### Preparation of N-[3(R)-[2-(6-Amino-2-pyridinyl) ethyl]-2-oxopiperidinyl]acetyl]-3(R)-methyl-β-alanine

Following the procedure of Duggan, et. al., J. Med. Chem. 1995, 38, 3332, except substituting (6-amino-2-pyridinyl)butanoic acid, Bondinell, et. al., WO 94/14775, for (N-Boc-piperidin-4-yl)butanoic acid, gives the title compound.

### Example 15

### Preparation of 3-[[[3-[2-(6-Aminopyrid-2-yl)ethyl]isoxazolin-5(R,S)-yl]acetyl]amino]-3(R,S)-methylpropanoic acid

a) 4-[6-(Toluenesulfonylamino)-2-pyridinyl]-1-butanoximinoyl chloride

Following the procedure of Example 1(b) in WO 95/14682, except substituting the compound of Preparation F(e) for the 4-cyanobenzoxime, the title compound is prepared.

b) tert-Butyl [3-[2-[(6-toluenesulfonylamino)-2-pyridinyl]ethyl]isoxazolin-5(R,S)-yl]acetate

Following the procedure of Example 1(d) in WO 95/14682, except substituting the compound of Example 15(a) for 4-cyanobenzoximinoyl chloride, and substituting tert-butyl 3-butenoate for methyl 3-butenoate, the title compound is prepared.

c) [3-[2-[(6-Toluenesulfonylamino)-2-pyridinyl]ethyl]isoxazolin-5(R,S)-yl]acetic acid

4M HCl in dioxane (10 mL) is added to a solution of the compound of Example 15(b) (5 mmol) in CH₂CH₂ (40 mL) at 0° C. The reaction is stirred at RT until complete, then is concentrated to afford the title compound.

d) Ethyl 3-[[3-[2-(6-aminopyrid-2-yl)ethyl]isoxazolin-5(R,S)-yl]acetyl]amino-3(R,S)-methylpropanoate

EDC (1.2 mmol) is added to a solution of the compound of Example 15(c) (1 mmol), ethyl 3(R,S)-aminobutyrate (1.2 mmol), HOBt.H₂O (1.2 mmol), and DIEA (4 mmol) in anhydrous CH₃CN (5 mL) at RT. The reaction is stirred at RT until complete, then is concentrated, and the residue is purified by chromatography (silica gel) to afford the tide compound.

e) 3-[[[3-[2-(6-Aminopyrid-2-yl)ethyl]isoxazolin-5(R,S)-yl]acetyl]amino]-3(R,S)-methylpropanoic acid

1.0N LiOH (2.5 mmol) is added to a solution of the compound of Example 15(d) (0.5 mmol) in THF (2.5 mL). The reaction is stirred at RT until complete, then is neutralized with 1.0N HCl. The solution is concentrated and the residue is purified by reverse-phase chromatography to afford the title compound.

### Example 16

### Preparation of N-8 3-[[[(6-Amino-2-pyridinyl)methyl]carbonyl]amino]benzoyl]-β-alanine

a) Benzyl N-[3-[[[(6-amino-2-pyridinyl)methyl]carbonyl]amino]benzoyl]-β-alaninate

A mixture of benzyl N-(3-aminobenzoyl-β-alaninate, Alig, et. al., EP 0372486, (1 mmol), the compound of Preparation G (1 mmol), EDC (1.5 mmol), and DIEA (3 mmol) in DMF (25 mL) is stirred at RT. The mixture is poured into 5% NaHCO₃ and extracted with EtOAc. The combined organic phase is washed with H₂O, dried (MgSO₄), and concentrated. The residue is chromatographed (silica gel) to give the title compound.

b) N-[3-[[[(6-Amino-2-pyridinyl)methyl]carbonyl]amino]benzoyl]-β-alanine

A mixture of the compound of Example 16(b)(1 mmol) and 1N NaOH (1.5 mL) in CH₃OH (20 mL) is stirred and concentrated. The residue is dissolved in H₂O, extracted with CH₂C1₂, and the aqueous phase is adjusted to pH 5 with dilute HCl to give the title compound.

### Example 17

### Preparation of [[1-[N-[[(6-Amino-2-pyridinyl))methyl]carbonyl]tyrosyl]-4-piperidinyl]oxy]acetic acid

a) tert-Butyl [[1-[N-[[(6-amino-2-pyridinyl)methyl]carbonyl]tyrosyl]-4-piperidinyl]oxy]acetate

A mixture of tert-butyl [(1-tyrosyl-4-piperidinyl)oxy]acetate, Alig, et. al., EP 372486, (1 mmol), the compound of Preparation G (1 mmol), EDC (1.5 mmol), and DIEA (3 mmol) in DMF (25 mL) is stirred at RT. The mixture is poured into 5% NaHCO₃ and extracted with EtOAc. The combined organic phase is washed with H₂O, dried (MgSO₄), and concentrated. The residue is chromatographed (silica gel) to give the title compound.

b) [[1-[N-[[(6-Amino-2-pyridinyl))methyl]carbonyl]tyrosyl]-4-piperidinyl]oxy]acetic acid

A mixture of the compound of Example 17(a)(1 mmol) and CF₃CO₂H in CH₂Cl₂ is stirred and concentrated to give the title compound.

### Example 18

### Preparation of (±)-3-[[[[2-(6-Aminopyrid-2-yl)ethyl]amino]succinoyl]amino]-4-pentynoic acid

a) Methyl 4-[[2-(6-aminopyrid-2-yl)ethyl]amino]-4-oxobutyrate

3-Carbomethoxypropionyl chloride (0.74 mL, 6.0 mmol) is added at 0° C. to a stirred solution of the compound of Preparation H (5.0 mmol) and DIEA (4.4 mL, 25 mmol) in dry CH₂Cl₂ (50 mL). After stirring for 1.5 hr at RT, the reaction mixture is diluted with CH₂Cl₂ (50 mL) and washed sequentially with H₂O (25 mL) and 5% NaHCO₃ (25 mL). The organic layer is dried (MgSO₄), concentrated, and reconcentrated from toluene. Chromatography (silica gel) gives the title compound.

b) 4-[[2-(6-Aminopyrid-2-yl)ethyl]amino]-4-oxobutyric acid

A mixture of the compound of Example 18(a) (530.6 mg, 2.0 mmol), 1.0N LiOH (3.0 mL, 3.0 mmol), THF (10 mL), and H₂O (7 mL) is stirred at RT overnight, then is concentrated to dryness. The residue is taken up in H₂O (5 mL) and neutralized with 1.0N HCl. The precipitate is collected and dried in vacuum to give the title compound.

c) Ethyl (±)-3-[[[[2-(6-aminopyrid-2-yl)ethyl]amino]succinoyl]amino]-4-pentynoate

EDC (230 mg, 1.2 mmol) is added to a solution of the compound of Example 18(b) (203.3 mg, 1.0 mmol), ethyl (±)-3-amino-4-pentynoate, WO 93/07867, (169.4 mg, 1.2 mmol), HOBt.H₂O (162.2 mg, 1.2 mmol), and DIEA (0.70 mL, 4 mmol) in anhydrous CH₃CN (5 mL) at RT. The reaction is stirred at RT overnight, then is concentrated to dryness. The residue is chromatographed (silica gel) to give the title compound.

d) (±)-3-[[[[2-(6-Aminopyrid-2-yl)ethyl]amino]succinoyl]amino]-4-pentynoic acid

A mixture of the compound of Example 18(c) (187.2 mg, 0.5 mmol), 1.0N LiOH (0.75 mL, 0.75 mmol), THF (2.5 mL), and H₂O (1.7 mL) is stirred at RT overnight, then is concentrated to dryness. The residue is taken up in H₂O (2 mL) and acidified with TFA. ODS chromatography followed by lyophilization of the purified material gives the title compound.

### Example 19

### Preparation of (S)-4-[[[(6-Amino-2-pyridinyl)methyl]carbonyl]glycyl]-3-methoxycarbonylmethyl-2-oxopiperazine-1-acetic acid

Following the procedure of Sugihara, et. al., EP 0529858, Example 59, except substituting the compound of Preparation G for 4-amidinobenzoic acid hydrochloride, gives the title compound.

### Example 20

### Preparation of (3S,5S)-5-[4-[(6-Amino-2-pyridinyl)methyl]phenyl]oxymethyl]-3-carboxymethyl-2-pyrrolidinone

a) (3S,5S)-5-[4-[(6-Amino-2-pyridinyl)methyl]phenyl]oxymethyl]-3-[(tert-butoxycarbonyl) methyl]-2-pyrrolidinone

Following the procedure of Himmelsbach, et al., AU-A-86926/91, Example 3(51), except substituting the compound of Preparation I for 4′-cyano-3′-fluoro-4-(hydroxy) biphenyl, gives the title compound.

b) (3S,5S)-5-[4-[(6-Amino-2-pyridinyl)methyl]phenyl]oxymethyl]-3-carboxymethyl-2-pyrrolidinone

Following the procedure of Himmelsbach, et. al., AU-A-86926/91, Example 7(93), except substituting the compound of Example 20(a) for (3S,5S)-3-[(tert-butyloxycarbonyl)methyl]-5-[(4′-amidino-3′-fluoro-4-biphenylyl)oxymethyl]-2-pyrrolidinone, gives the title compound.

### Example 21

### Preparation of 1-[(6-Amino-2-pyridinyl)methyl]-3-[4-(2-carboxyethyl)phenyl]-4-methoxy-3-pyrrolin-2-one

Following the procedures of Linz, et. al., EP 0567968, except substituting the compound of Preparation U for 4-cyanoaniline, gives the title compound.

### Example 22

### Preparation of 4-[[[(6-Amino-2-pyridinyl)methyl]methylamino]acetyl]phenoxyacetic acid

a) Methyl 4-[[[(6-amino-2-pyridinyl)methyl]methylamino]acetyl]phenoxyacetate

Following the procedure of Wayne et. al., WO 94/22834, Example 1, except substituting the compound of Preparation T (1 mmol) for 1-(4-pyridyl)piperazine, gives the title compound.

b) 4-[[[(6-Amino-2-pyridinyl)methyl]methylamino]acetyl]phenoxyacetic acid

Following the procedure of Wayne et. al., WO 94/22834, Example 2, except substituting the compound of Example 22(a) for methyl 4-[2-[4-(4-pyridinyl)piperazin-1-yl]acetyl]phenoxyacetate, gives the title compound.

### Example 23

### Preparation of 2,2′-[[4-[[[(6-Amino-2-pyridinyl)methyl]methylamino]acetyl]-1,2-phenylene]bis(oxy)]bis-acetic acid

a) Dimethyl 2,2′-[[-4-[[[(6-amino-2-pyridinyl)methyl]methylamino]acetyl]-1,2-phenylene]bis(oxy)]bis-acetate

Following the procedure of Wayne et. al., WO 94/22834, Example 3, except substituting the compound of Preparation T for 1-(4-pyridyl)piperazine, gives the title compound.

b) 2,2′-[[4-[[[(6-Amino-2-pyridinyl)methyl]methylanino]acetyl]-1,2-phenylene]bis(oxy)]bis-acetic acid

Following the procedure of Wayne et. al., WO 94/22834, Example 4, except substituting the compound of Example 23(a) for dimethyl 2,2′-[4-[2-4-(4-pyridinyl)piperazin-1-yl)acetyl]phenylene-1,2-dioxy]diacetate, gives the title compound.

### Example 24

### Preparation of 4-[[[[(6-Amino-2-pyridinyl)methyl]carbonyl]methylamino]acetyl]phenoxyacetate

a) Benzyl 4-[[[[(6-amino-2-pyridinyl)methyl]carbonyl]methylamino]acetyl]phenoxyacetate

A mixture of the compound of Preparation J(c)(1 mmol), Preparation G (1 mmol), EDC (1.5 mmol), and DIEA (3 mmol) in DMF (25 mL) is stirred at RT. The mixture is poured in to 5% NaHCO₃ and extracted with EtOAc. The organic phase is washed with H₂O, dried (MgSO₄), and concentrated. The residue is chromatographed (silica gel) to give the tide compound.

b) 4-[[[[(6-Amino-2-pyridinyl)methyl]carbonyl]methylamino]acetyl]phenoxyacetate

The compound of Example 24(a)(1 mmol) and 1N NaOH (1.5 mL) in CH₃OH (20 mL) is stirred and concentrated. The residue is dissolved in H₂O, extracted with CH₂Cl₂, and the aqueous phase is adjusted to pH 5 with dilute HCl to give the title compound.

### Example 25

### Preparation of 4-[[[[(6-Amino-2-pyridinyl methyl]carbonyl]methylamino]acetyl]-1,2-phenylenedioxydiacetic acid

a) Dimethyl 4-[[[[(6-amino-2-pyridinyl)methyl]carbonyl]methylamino]acetyl]-1,2-phenylenedioxydiacetate

Following the procedure of Example 24(a), except substituting the compound of Preparation K(c) for the compound of Preparation J(c), gives the title compound.

b) 4-[[[[(6-Amino-2-pyridinyl)methyl]carbonyl]methylamino]acetyl]-1,2-phenylenedioxydiacetic acid

Following the procedure of procedure of Example 24(b), except substituting the compound of Example 25(a) for the compound of Example 24(a), gives the title compound.

### Example 26

### Preparation of 1-[(2-Amino-6-pyridinyl)methyl]-3-[4[2-(carboxy)ethyl)]phenyl]-3-oxo-imidazolidine

a) Ethyl 2-[4-(2-hydroxyethylamino)phenyl]propionate

Following the procedure of Himmelsbach, et. al., EP 0587134, Example V, glycolaldehyde dimer (Aldrich) (1 mmol) is added to a solution of methyl 2-(4-aminophenyl)propionate, (1 mmol) in aqueous CH₃CN (pH 6-7) (10 mL), followed by NaBH₃CN (1.2 mmol), and the mixture is allowed to stir for 1 h. The mixture is concentrated to an oil, and the residue is dissolved in a mixture of ice water and EtOAc. The aqueous layer is neutralized with 4N NaOH and washed with EtOAc. The organic phase is concentrated and a solution of the resulting oil in EtOAc is chromatographed (silica gel, gradient, 5-30% CH₃OH/CH₂Cl₂-0.1% HOAc). The fractions containing the product are combined and concentrated to give the title compound.

b) N-[(2-Phthaloyl-6-pyridinyl)methyl]-N′-hydroxyethyl-N′-[4[(2-ethoxycarbonyl)ethyl)]phenyl]-urea

Following the procedures of Himmelsbach, et. al., EP 0587134 and EP 0612741, a solution of the compound of Preparation L (1 mmol) and COCl₂ (1.1 mmol) in TBF (20 mL) is allowed to stir at −20° C. for 20 min. The compound of Example 26(a) (1 mmol) is added to the solution and the resulting mixture is allowed to stir and warm RT for 18 h. The resulting solution is concentrated and a solution of the resulting residue in EtOAc is washed with 5% citric acid followed by H₂O. The organic phase is concentrated and a solution of the resulting oil in EtOAc is chromatographed (silica gel, gradient, 5-30% CH₃OH/CH₂Cl₂-0.1% HOAc). The fractions containing the product are combined and concentrated to give the title compound.

c) N¹-[(2-Phthaloyl-6-pyridinyl)methyl]-N³-[4[(2-ethoxycarbonyl)ethyl)]phenyl]-2-oxo-imidazolidine

Following the procedures of Himmelsbach, et. al., EP 0587134, Example III, and EP 0612741, a solution of the compound of Example 26(b) (1 mmol), methanesulfonyl chloride (1.2 mmol) and Et₃N (1.2 mmol) in CH₂Cl₂ (5 mL) is allowed to stir at 0° C. for 1 h. The mixture is partitioned between H₂O and CH₂Cl₂. The organic phases are combined, dried (Na₂SO₄), and concentrated. A solution of the residue and NaI (1.1 mmol) in acetone (5 mL) is heated to reflux for 3 h and then concentrated. Potassium bis(trimethylsilyl)azide (1.2 mmol) is added to a solution of the resulting oil in DMF (5 mL), cooled to 0° C. The solution is allowed to warm to RT over 30 min and concentrated to give an oil. The oil is partitioned between H₂O and CH₂Cl₂. The organic phases are combined, dried (Na₂SO₄), and concentrated. A solution of the resulting oil in EtOAc is chromatographed (silica gel, gradient, 5-30% CH₃OH/CH₂Cl₂-0.1% HOAc). The fractions containing the product are combined and concentrated to give the title compound.

d) N¹-[(Amino-6-pyridinyl)methyl]-N³-[4[(2-carboxyl)ethyl)]phenyl]-2-oxo-imidazolidine

Following the procedures of Himmelsbach, et. al, EP 0587134, Example III, and EP 0612741, a solution of the compound of Example 26(c) (1 mmol) and hydrazine hydrate (1.1 mmol) in EtOH (10 mL) is allowed to stir for 18 h. The solution concentrated and the residue is partitioned between H₂O and EtOAc. The organic phases are combined and concentrated. A solution of the resulting oil in THF (5 mL) and 1N NaOH (1.2 mL, 1.2 mmol) is allowed to stir for 18 h. The mixture is neutralized with conc HCl and chromatographed (silica gel, gradient, 5-30% CH₃OH/CH₂Cl₂-0.1% HOAc). The fractions containing the product are combined and concentrated to give the title compound.

### Example 27

### Preparation of [6-[[[(6-Amino-2-pyridinyl)methyl]methylamino]carbonyl]-1,2,3,4-tetrahydroisoquinolin-2-yl]acetic acid

a) Ethyl [6-[[[(6-amino-2-pyridinyl)methyl]methylamino]carbonyl]-1,2,3,4-tetrahydroisoquinolin-2-yl]acetate

A solution of the compound of Preparation M(d) (0.263 g, 1.0 mmol), the compound of Preparation T (0.34 g, 1.0 mmol), EDC (0.191 g, 1.0 mmol), HOBt (0.151 g, 1.0 mmol) and Et₃N (0.235 mL, 2.0 mmol) in DMF (7 mL) is stirred for 8 h. The solution is concentrated to an oil which is purified by chromatography (silica gel, gradient, 10-33% CH₃OH/CH₂Cl₂) to afford the title compound (0.32 g, 77%)

b) [6-[[[(6-Amino-2-pyridinyl)methyl]methylamino]carbonyl]-1,2,3,4-tetrahydroisoquinolin-2-yl]acetic acid

A solution of the compound of Example 27(a) (0.42 g, 1.0 mmol) in 1N NaOH (1.5 mL, 1.5 mmol) and EtOH (5 mL) is stirred for 8 h. The solution is concentrated to an oil which is purified by chromatography (silica gel, gradient, 10-33% CH₃OH(CH₂Cl₂) to afford the title compound (0.35 g, 76%).

### Example 28

### Preparation of [6-[[[(6-Amino-2-pyridinyl)methyl]methylamino]carbonyl]-1,2,3,4-tetrahydro-1-oxo-isoquinolin-2-yl]acetic acid

a) Ethyl [6-[[[(6-Amino-2-pyridinyl)methyl]methylamino]carbonyl]-1,2,3,4-tetrahydro-1-oxo-isoquinolin-2-yl)acetate

Following the procedure of Example 27(a), except substituting the compound of Preparation N(d) for the compound of Preparation M(d), gives the title compound.

Alternatively, a solution of the compound of Preparation N(c) (0.23 g, 1.0(mmol), Pd(OAc)₂ (0.026 g, 0.1 mmol), the compound of Preparation T (0.31 g, 1.0 mmol), Ph₃P (0.262 g, 1.0 mmol), diisopropylamine (0.25 mL, 2.1 mmol), and NMP (7 mL) in 10% NH₄CO₃ is stirred for 8 h under an atmosphere of CO. The solution is concentrated to an oil which is purified by chromatography (silica gel, gradient, 25-75% CH₃OH/CH₂Cl₂) to afford the title compound(0.26 g, 76%)

b) [6-[[[(6-Amino-2-pyridinyl)methyl]methylamino]carbonyl]-1,2,3,4-tetrahydro-1-oxo-isoquinolin-2-yl]acetic acid

Following the procedure of Example 27(b), except substituting the compound of Example 28(a) for the compound of Example 27(a), gives the title compound.

### Example 29

### Preparation of [6-[[[(6-Amino-2-pyridinyl)methyl]carbonyl]amino]tetralin-2-yl]acetic acid

a) tert-Butyl [6-[[[[(6-Amino-2-pyridinyl)methyl]carbonyl]amino]tetralin-2-yl]acetate

Following the procedure of Example 27(a), except substituting tert-butyl (6-amino-tetralin-2-yl)acetate [Fisher, et. al., EO 0635492, Scheme 12 and Example 28, parts A-D] for the compound of Preparation M(d) and substituting the compound of Preparation G for the compound of Preparation T, gives the title compound.

b) [6-[[[[(6-Amino-2-pyridinyl)methyl]carbonyl]amino]tetralin-2-yl]acetic acid

A solution of the compound of Example 29(a) (0.32 g, 1.0 mmol) and TFA (5 mL) in CH₂Cl₂ (5 mL is allowed to stir for 1 h. The solution is concentrated to an oil which is treated with Et₂O. Filtration and drying in vacuo afforded the title compound (0.15 g, 50%)

### Example 30

### Preparation of [6-[[[[(6-Amino-2-pyridinyl)methyl]methylamino]carbonyl]tetralin-2-yl]acetic acid

Following the procedure of Example 27, except substituting the compounds of Preparation O(b) for the compound of Preparation M(d), gives the title compound.

### Example 31

### Preparation of [5-[[[[(6-Amino-2-pyridinyl)methyl]carbonyl]amino]benzofuran-2-yl]propionic acid

Following the procedure of Example 27, except substituting ethyl (5-aminobenzofuran-2-yl)propionate from Preparation P(e) for the compound of Preparation M(d), gives the title compound.

### Example 32

### Preparation of [5-[[[[(6-Amino-2-pyridinyl)methyl]carbonyl]amino]-2,3-dihydro-benzofuran-2-yl]propionic acid

Following the procedure of Example 27, except substituting ethyl (5-amino-2,3-dihydro-benzofuran-2-yl)propionate from Preparation P(e) for the compound of Preparation M(d), gives the title compound.

### Example 33

### Preparation of [5-[[[[(6-Amino-2-pyridinyl)methyl]methylamino]carbonyl]benzofuran-2-yl]propionic acid

Following the procedure of Example 28, except substituting the compounds of Preparation Q(d) or (e) for the compounds of Preparation N(c) or (d), gives the title compound.

### Example 34

### Preparation of [5-[[[[(6-Amino-2-pyridinyl)methyl]methylamino]carbonyl]-2,3-dihydro-benzofuran-2-yl]-propionic acid

Following the procedure of Example 28, except substituting the compounds of Preparation R(a) or (b) for the compounds of Preparation N(c) or (d), gives the title compound.

### Example 35

### Preparation of (±)-3-[[[4-(6-Amino-2-pyridinyl)butanoyl]glycyl]amino]-4-pentynoic acid

a) Ethyl (±)-3-[[[(6-Amino-2-pyridinyl)butanoyl]glycyl]amino]4-pentynoate

DIEA (5.43 mmol) is added to a stirred solution of the compound of Preparation S(b) (1.76 mmol), 4-(6-amino-2-pyridinyl)butyric acid, Bondinell, et al., WO 94/14775, (1.55 mmol), HOBt.H₂O (2.33 mmol), and EDC (2.33 mmol) in anhydrous CH₃CN (15 mL) at RT. The reaction mixture is stirred, concentrated, diluted with CH₂Cl₂ (100 mL), and washed sequentially with 5% NaHCO₃ and brine. Drying (MgSO₄), concentration, and chromatography (silica gel, CH₃OH(CH₂Cl₂) gives the title compound.

b) (±)-3-[[[4-(6-Amino-2-pyridinyl)butanoyl]glycyl]amino]4-pentynoic acid

1.0N LiOH (0.71 mmol) is added dropwise at RT to a mixture of the compound of Example 35(a) (0.285 mmol) in TBF (5 mL), H₂O (5 mL) and CH₃CN (1 mL). The mixture is stirred, concentrated to a small volume, and cooled in an ice bath before neutralizing with 1.0N AcOH (0.70 mL). The solution is lyophilized and the residue is purified by chromatography (ODS, CH₃CN/H₂O-0.1% TFA) to give the title compound.

The above description fully discloses how to make and use the present invention. However, the present invention is not limited to the particular embodiments described hereinabove, but includes all modifications thereof within the scope of the following claims. The various references to journals, patents and other publications which are cited herein comprises the state of the art and are incorporated herein by reference as though fully set forth.

What is claimed is:

1. A compound according to formula (I): 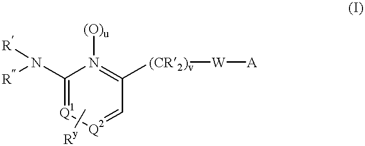
wherein
A is a fibrinogen antagonist template;
W is a linking moiety of the form —(CHR^{g})ₐ—U—(CHR^{g})_{b}—V—;
Q¹, Q² and Q³ are independently N or C—R^{y}, provided that no more than one of Q¹, Q² and Q³ is N;
R′ is is H or C₁₋₆alkyl, C₃₋₇cycloalkyl-C₀₋₆alkyl or Ar—C₀₋₆alkyl
R″ is R′, —C(O)R′ or —C(O)OR′;
R^{g} is H or C₁₋₆alkyl, Het-C₀₋₆alkyl, C₃₋₇cycloalkyl-C₀₋₆alkyl or Ar—C₀₋₆alkyl;
R^{k} is R^{g}, —C(O)R^{g} or —C(O)OR^{g}
Rⁱ is H, C₁₋₆alkyl, Het-C₀₋₆alkyl, C₃₋₇cycloalkyl-C₀₋₆alkyl, Ar—C₀₋₆alkyl, Het-C₀₋₆alkyl-U′-C₁₋₆alkyl, C₃₋₇cycloalkyl-C₀₋₆alkyl-U′-C₁₋₆alkyl or Ar—C₀₋₆alkyl-U′-C₁₋₆alkyl;
R^{y} is H, halo, —OR^{g}, —SR^{g}, —CN, —NR^{g}R^{k}, —NO₂, —CF₃, CF₃S(O)ᵣ—, —CO₂R^{g}, —COR^{g} or —CONR^{g}₂;
U and V are absent or CO, CR^{g}₂, C(═CR^{g}₂), S(O)_{c}, O, NR^{g}, CR^{g}OR^{g}, CR^{g}(OR^{k})CR^{g}₂, CR^{g}₂CR^{g}(OR^{k}), C(O)CR^{g}₂, CR^{g}₂C(O), CONRⁱ, NRⁱCO, OC(O), C(O)O, C(S)O, OC(S), C(S)NR^{g}, NR^{g}C(S), S(O)₂NR^{g}, NR^{g}S(O)₂ N═N, NR^{g}NR^{g}, NR^{g}CR^{g}₂, NR^{g}CR^{g}₂, CR^{g}₂O, OCR^{g}₂, CR^{g}═CR^{g}, C≡C, Ar or Het;
a is 0, 1 or 2;
b is 0, 1 or 2;
c is 0, 1 or 2;
r is 0, 1 or 2;
u is 0 or 1; and
v is 0 or ;
or pharmaceutically acceptable salts thereof;
provided that:
(i) when v is 0, and R′, R″ and R^{y} are H, and Q¹-Q³ are CH, W-A is not 7-aminocarbonyl-2,3,4,5-tetrahydro-3-oxo-4-methyl-1H-1,4-benzodiazepine-2-acetic acid, 7-aminocarbonyl-1-acetyl-2,3,4,5-tetrahydro-3-oxo-4-methyl-1H-1,4-benzodiazepine-2-acetic acid, or 7-aminocarbonyl-2,3,4,5-tetrahydro-3-oxo-4-methyl-1H-1-benzazepine-2-acetic acid, or the methyl esters thereof;
(ii) when v is 0 or 1 and R′, R″ and R^{y} are H, and Q¹-Q³ are CH, W-A is not 3-propanoyl-glycyl-aspartyl-phenylalanine, or 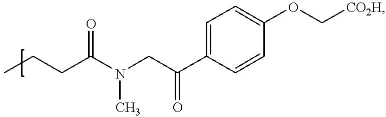
and the benzyl esters thereof.

2. A compound according to claim 1 in which Q¹, Q² and Q³ are each CH, and u is 0.

3. A compound according to claim 1 in which R′ is H and R″ is H or C₁₋₄alkyl.

4. A compound according to claim 1 in which W is —(CHR^{g})ₐ—CONRⁱ— or —(CHR^{g})ₐ—NRⁱCO—.

5. A compound according to formula (I) in which A is chosen from the group of 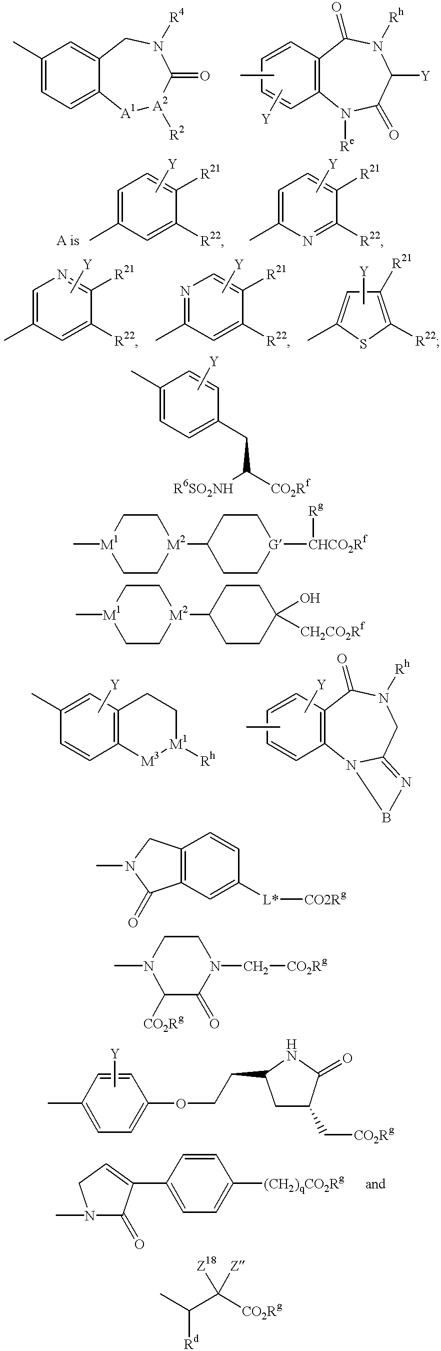
and which has 13-14 covalent bonds between the acidic moiety and the first nitrogen in the pyridine ring.

6. A compound according to claim 1 in which is: 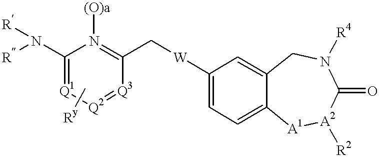
wherein
A¹-A² is NR¹—CH, NC(O)R³—CH, N═C, CR¹═C, CHR¹—CH, O—CH or S—CH;
R¹ is H, C₁₋₆ alkyl or benzyl;
R² is (CH₂)_{q}CO₂H;
R⁴ is H, C₁₋₆alkyl, Ar—C₀₋₆alkyl, Het-C₀₋₆alkyl, or C₃₋₆cycloalkyl-C₀₋₆alkyl; and
q is 1, 2 or 3.

7. A compound according to claim 6 wherein A¹-A² is NH—CH and R² is CH₂CO₂H.

8. A compound according to claim 7 wherein W is —(CHR^{g})ₐ—CONRⁱ— or —(CHR^{g})ₐ—NRⁱCO—.

9. A compound according to claim 1 which is:
3-[3,4-Dihydro-8-[[[(6-amino2-pyridinyl)methyl]methylamino]carbonyl]-1-methyl-2,5-dioxo-1H-1,4-benzodiazepine]4-propanoic acid;
3-[4H-imidazo[1,2-a][1,4]benzodiazepine-5(6H)-1-methyl-6oxo-9-[[[(6-amino-2-pyridinyl) methyl]methylamino]carbonyl]-5-propanoic acid;
4-[4-[2-(6-Amino-2-pyridinyl)ethyl]-1-piperazinyl]-1-piperidineacetic acid;
1-Hydroxyl-4-[4-[(6-amino-2-pyridinyl)methyl]-1-piperazinyl]-cyclohexaneacetic acid;
1-Hydroxyl-4-[4-[2-(6-amino-2-pyridinyl)ethyl]-1-piperazinyl]-cyclohexaneacetic acid;
4-[4-[(6-Amino-2-pyridinyl)methyl]-1-piperazinyl]-1-piperidineacetic acid;
N-[[1-[[2-(6-Amino-2-pyridinyl)ethyl]carbonyl]-3-piperidinyl]carbonyl]-b-alanine;
2-[(6-Amino-2-pyridinyl)methyl]-5-[2-(carboxy-ethyl)amino]carbonyl]; -2,3-dihydro-3-oxo-1H-isoindole;
(S)-2-(Butylsulfonylamino)-3-[4-[[3-(6-amino-2-pyridinyl)propyl]oxy]phenyl]propionic acid;
N-[3(R)-[2-(6-Amino-2-pyridinyl)ethyl]-2-oxopiperidinyl]acetyl]-3(R)-methyl-b-alanine;
3-[[[3-[2-(6-Aminopyrid-2-yl)ethyl]isoxazolin-5(R,S)-yl]acetyl]amino]-3(R,S)-methylpropanoic acid;
N-[3-[[[(6-Amino-2-pyridinyl)methyl]carbonyl]amino]benzoyl]-b-alanine;
[[1-[N-[[(6-Amino-2-pyridinyl))methyl]carbonyl]tyrosyl]-4-piperidinyl]oxy]acetic acid;
(±)-3-[[[[2-(6-Aminopyrid-2-yl)ethyl]amino]succinoyl]amino]-4-pentynoic acid;
(S)-4-[[[(6-Amino-2-pyridinyl)methyl]carbonyl]glycyl]-3-methoxycarbonylmethyl-2-oxopiperazine-1-acetic acid;
(3S,5S)-5-[4-[(6-Amino-2-pyridinyl)methyl]phenyl]oxymethyl]-3-carboxymethyl-2-pyrrolidinone;
1-[(6-Amino-2-pyridinyl)methyl]-3-[4-(2-carboxyethyl)phenyl]-4-methoxy-3-pyrrolin-2-one;
4-[[[(6-Amino-2-pyridinyl)methyl]methylamino]acetyl]phenoxyacetic acid;
2,2′-[[4-[[[(6-Amino-2-pyridinyl)methyl]methylamino]acetyl]-1,2-phenylene]bis(oxy)]bis-acetic acid;
4-[[[[(6-Amino-2-pyridinyl)methyl]carbonyl]methylamino]acetyl]phenoxyacetate;
4-[[[[(6-Amino-2-pyridinyl)methyl]carbonyl]methylamino]acetyl]-1,2-phenylenedioxydiacetic acid;
1-[(2-Amino-6-pyridinyl)methyl]-3-(4[2-(carboxy)ethyl)]phenyl]-3-oxo-imidazolidine;
[6-[[[(6-Amino-2-pyridinyl)methyl]methylamino]carbonyl]-1,2,3,4-tetrahydroisoquinolin-2-yl]acetic acid;
[6-[[[(6-Amino-2-pyridinyl)methyl]methylamino]carbonyl]-1,2,3,4-tetrahydro-1-oxo-isoquinolin-2-yl]acetic acid;
[6-[[[[(6-Amino-2-pyridinyl)methyl]carbonyl]amino]tetralin-2-yl]acetic acid;
[6-[[[[(6-Amino-2-pyridinyl)methyl]methylamino]carbonyl]tetralin-2-yl)acetic acid;
[5-[[[[(6-Amino-2-pyridinyl)methyl]carbonyl]amino]benzofuran-2-yl]propionic acid;
[5-[[[[(6-Amino-2-pyridinyl)methyl]carbonyl]amino]-2,3-dihydro-benzofuran-2-yl]propionic acid;
[5-[[[[(6-Amino-2-pyridinyl)methyl]methylamino]carbonyl]benzofuran-2-yl]propionic acid;
[5-[[[[(6-Amino-2-pyridinyl)methyl]methylamino]carbonyl]-2,3-dihydro-benzofuran-2-yl]-propionic acid; or
(±)-3-[[[4-(6-Amino-2-pyridinyl)butanoyl]glycyl]amino]-4-pentynoic acid.

10. A compound according to claim 1 which is:
(S)-7-[[[(6-Amino-2-pyridinyl)methyl]methylamino]carbonyl]-2,3,4,5-tetrahydro-4-methyl-3-oxo-1H-1,4-benzodiazepine-2-acetic;
(S)-7-[[[(6-Amino-2-pyridinyl)methyl]amino]carbonyl]-2,3,4,5-tetrahydro-4-methyl-3-oxo-1H-1,4-benzodiazepine-2-acetic acid;
(S)-7-[[[(6-Ethylamino-2-pyridinyl)methyl]amino]carbonyl]-2,3,4,5-tetrahydro-4-methyl-3-oxo-1H-1,4-benzodiazepine-2-acetic acid; or
(±)-7-[[[(2-Amino-4-pyrimidinyl)methyl]methylamino]carbonyl]-2,3,4,5-tetrahydro-4-methyl-3-oxo-1H-1,4-benzodiazepine-2-acetic acid.

11. A pharmaceutical composition which comprises a compound according to any one of claims 1-10 and a pharmaceutically acceptable carrier.

12. A method of treating a disease state in which antagonism of the vitronectin receptor is indicated which comprises administering a compound according to claim 1.

13. A method according to claim 12 for inhibiting angiogenesis or treating atherosclerosis, restenosis, inflammation, cancer or osteoporosis.

14. The use of a compound according to any one of claims 1-10 in the manufacture of a medicament.

15. The use of a compound according to any one of claims 1-10 in the manufacture of a medicament for the inhibition of the vitronectin receptor in a mammal in need thereof.

16. The use of a compound according to any one of claims 1-10 in the manufacture of a medicament for the treatment of atherosclerosis, restenosis, inflammation, cancer or osteoporosis.

17. A process for preparing a compound of formula (I) as defined in claim 1, which process comprises reacting a compound of formula (XVI) with a compound of formula (XVII): 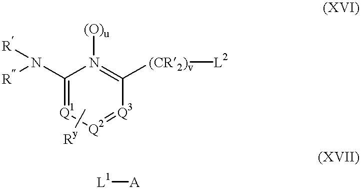
wherein:
Q¹, Q², Q³, R^{y}, R′, R″, u, v and A are as defined in formula (I), with any reactive functional groups protected; and
L¹ and L² are groups which react to form a covalent bond in the moiety W as defined in formula (I);
and thereafter removing any protecting groups, and optionally forming a pharmaceutically acceptable salt.
